(19) United States
(12) Patent Application Publication
(10) Pub. No.: US 2002/0183316 A1
(43) Pub. Date: Dec. 5, 2002

(54) Amidoalkyl-piperidine and amidoalkyl-piperazine derivatives useful for the treatment of nervous system disorders

(76) Inventors: Kevin Pan, Phoenixville, PA (US); Michael H. Parker, Chalfont, PA (US); Allen B. Reitz, Lansdale, PA (US); Steven J. Coats, Quakertown, PA (US); Cheryl P. Kordik, Lansdale, PA (US); Chi Luo, New Hope, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/001,725

(22) Filed: Oct. 22, 2001

Related U.S. Application Data
(60) Provisional application No. 60/244,117, filed on Oct. 27, 2000.

(51) Int. Cl. C07D 413/14; C07D 417/14; C 07D 4 3/02; A61K 31/5377; A61K 31/496; A61K 31/54

(52) U.S. Cl. 514/227.8; 514/235.5; 514/235.8; 514/252.12; 514/253.01; 514/254.01; 514/315; 514/326; 544/60; 544/120; 544/129; 544/360; 544/358; 544/372; 546/192; 546/208

(57) ABSTRACT

Novel amidoalkyl-piperidine and amidoalkyl-piperazine derivatives of the general formula

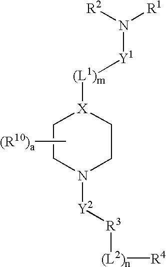

wherein all variables are as described herein, useful in the treatment of disorders, such as depression, dementia, schizophrenia, bipolar disorders, anxiety, emesis, acute or neuropathic pain, itching, migraine and movement disorders.

### FIELD OF THE INVENTION

The present invention is directed to novel amidoalkyl-piperidine and amidoalkyl-piperazine derivatives, pharmaceutical compositions containing them and their use in the treatment of nervous system disorders such as depression, dementia, anxiety, bipolar disorder, schizophrenia, emesis, migraine, itching, acute pain, neuropathic pain and movement disorders.

### BACKGROUND OF THE INVENTION

Current pharmacological therapies for the treatment of anxiety disorders include benzodiazepines, serotonin receptor modulators, SSRI (selective serotonin re-uptake inhibitors) and others. None of these drug classes is considered ideal, for a variety of reasons. Benzodiazepines are the most commonly prescribed drugs for anxiety; they offer excellent efficacy and a rapid onset of action, but may cause cognitive impairment, interference with daily activities, and have a significant potential for dependency and abuse. Serotonin receptor modulators, such as the azaperones, are well tolerated, but are not as efficacious as the benzodiazepines. The SSRIs are effective in alleviating symptoms of depression and anxiety and are well tolerated, but have a longer delayed onset of action than the benzodiazepines.

The ideal agent for treating anxiety disorders would be one which would treat the underlying pathophysiology of anxiety disorders. It would offer a rapid onset of action and would effectively alleviate the symptoms of anxiety, as well as panic disorder. The ideal agent would also effectively treat specific anxiety disorders such as post-traumatic stress disorder or generalized anxiety disorder. It would have an excellent side effect profile and a low potential for dependency, abuse and drug interactions.

The currently available pharmacological treatment options for depression, including serotonin modulators, SSRIs, tricyclic antidepressants and monoamine oxidase inhibitors, are also not considered ideal. Selective serotonin re-uptake inhibitors, tricyclic antidepressants, and monoamine oxidase inhibitors are the most commonly prescribed; they offer good efficacy, but have a slow onset of action and significant side effects. Serotonin receptor modulators such as the azaperones are well tolerated, but have been shown to yield only a modest antidepressant effect in the clinic. Although SSRIs are generally well tolerated and are effective in alleviating the symptoms of depression and anxiety, SSRIs are often associated with significant side effects such as sexual dysfunction and body weight gain, often resulting in noncompliance and self-discontinuation. Based on early clinical studies, neurokinin-1 receptor antagonists are expected to have a relatively rapid onset of pharmacological action, as well as low potential for side effects.

The ideal antidepressant agent would be one which would treat the underlying pathophysiology of affective disorders. It would offer a rapid onset of action and would effectively alleviate the symptoms of depression. It would have an excellent side effect profile and a low potential for dependency, abuse and drug interactions. It would lack sedation, anticholinergic effects, cardiovascular liabilities, proconvulsant activity, and would not induce body weight increase or sexual dysfunction.

The effectiveness of chemical compounds for the treatment of anxiety disorders and/or depression can be determined via in vivo testing. More particularly, the effectiveness of a chemical compound for the treatment of anxiety disorders and/or depression can be determined by measuring the behavioral effect (head shake) induced by 1-[2,5-dimethoxy-4-iodophenyl]-2-aminopropane (DOI), a drug with high affinity as an agonist for 5-HT_{2A/2c} receptors (Willins, D. L. and Meltzer, H. Y. J. Pharmacol. Exp. Ther. (1997), 282 pp 699-706), in mice treated with the chemical compound as compared with mice treated with vehicle. This in vivo assay is particularly useful because it is sensitive to drugs which modulate serotonin pathways, either directly or indirectly. (Sibille, E., et al in Mol. Pharmacol. (1997), 52 pp1056-1063 disclosed that antidepressants act by down-regulating of the 5-HT_{2A} and 5-HT_{2c} receptors, and that antisense inhibition in mice is associated with antidepressant effects.) Thus compounds that inhibit head shake would be expected to have therapeutic utility in the treatment of psychiatric disorders including depression, anxiety and schizophrenia.

An alternative, widely employed, in vivo test for determining the efficacy of a chemical compound for the treatment of anxiety disorders and/or depression is the elevated plus maze (EPM). The fully quantitative computerized EPM has validity as an anxiety model from the theoretical basis and the pharmacological responses of known anxiolytics. The EPM also has high ecological validity, since it measures the spontaneous behavioral patterns in response to interactions with the environment. The procedure for the EPM assay is based on the natural aversion of rodents to explore open and high places, as well as their innate tendency for thigmotaxis. When rats are placed on the elevated-plus maze, they have a normal tendency to remain in the enclosed arms of the maze and avoid venturing into the open arms. Animals treated with typical or atypical anxiolytics show an increase in the percentage of time spent (% Time) and/or the percentage of entries made (% Entries) into the open arms. Therefore, compounds which induce an increase in the % Time and/or % Entries relative to vehicle would be expected to have therapeutic utility in the treatment of psychiatric disorders including depression and anxiety.

Shue, et al., in U.S. Pat. No. 5,892,039 disclose piperazine derivatives useful as neurokinin antagonists for the treatment of chronic airway diseases such as asthma. Take, et al., in PCT Application WO 00/35915 disclose piperazine derivatives useful for treating and preventing Tachykinin-mediated diseases.

Himmelsbach et al., in EP496378, U.S. Pat. No. 5,597,825, U.S. Pat. No. 5,736,559 and U.S. Pat. No. 5,922,763 disclose biphenyl derivatives which have aggregation-inhibiting effects. Franckowiak et al., in U.S. Pat. No. 4,753,936 disclose a series of 1,4-dihydropyridine-3-carboxylic acid piperazine as circulation-active compounds. Mase, et al in EP350154 disclose a series of pyridylthiazolidine carboxamide derivative which have anti-PAF activity, useful in the treatment of asthma, inflammation, thrombosis, shock and other disorders. Takasugi, et al., in EP377457 disclose thiazole compounds which possess antithrombic, vasodilating, antiallergic, antiinflammatory and 5-lipoxygenase inhibitory activity.

### SUMMARY OF THE INVENTION

The present invention is directed to novel amidoalkyl-piperidine and amidoalkyl-piperazine derivatives, pharmaceutical compositions containing them and their use in the treatment of nervous system disorders such as depression, dementia, anxiety, bipolar disorder, schizophrenia, emesis, migraine, itching, acute pain, neuropathic pain and movement disorders.

More particularly, the present invention is directed to compounds of the formula (I)

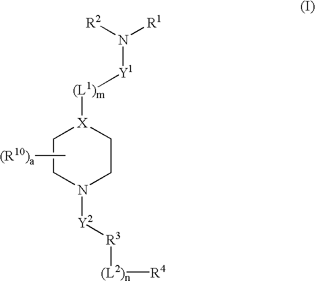

wherein

a is an integer selected from 0 to 2;

R¹⁰ is selected from the group consisting of C₁₋₆alkyl, aryl, C₃-C₈cycloalkyl, aralkyl, heteroaryl, heteroaryl-C₁₋₆alkyl, heterocycloalkyl and heterocycloalkyl-C₁₋₆alkyl; wherein the aryl, cycloalkyl, aralkyl, heteroaryl or heterocycloalkyl group may be optionally substituted with one to four substituents independently selected from halogen, hydroxy, C₁₋₆alkyl, halogenatedC₁₋₆alkyl, C₁₋₆alkoxy, halogenatedC₁₋₆alkoxy, nitro, cyano, amino, C₁₋₄alkylamino, di(C₁₋₄alkyl)amino, C₁₋₆alkylsulfonyl, C₁₋₆alkoxysulfonyl or halogenated C₁₋₆alkylsulfonyl;

X is selected from the group consisting of CH, C(C₁-C₆alkyl) and N;

m is an integer selected from 0 and 1;

L¹ is selected from the group consisting of C₁-C₆alkyl;

Y¹ is selected from the group consisting of C(O) and C(S);

R¹ and R² are each independently selected from the group consisting of hydrogen, C₁-C₆alkyl, aryl, aralkyl, C₃-C₈cycloalkyl, C₃-C₈cycloalkyl-C₁₋₆alkyl, heteroaryl, heteroaryl-C₁₋₆alkyl, heterocycloalkyl and heterocycloalkyl-C₁₋₆alkyl; wherein the aryl, aralkyl, cycloalkyl, heteroaryl or heterocycloalkyl may be optionally substituted with one or more substituents independently selected from halogen, hydroxy, C₁-C₆alkyl, C₁-C₆alkoxy, halogenatedC₁-C₆alkyl, halogenatedC₁-C₆alkoxy, nitro, cyano, amino, C₁-C₄alkylamino, di(C₁-C₄alkyl)amino, heteroaryl or heterocycloalkyl;

alternatively, R¹ and R² may be taken together with the nitrogen atom to which they are bound to form a five to six membered monocyclic ring structure selected from the group consisting of pyrrolidinyl, piperidinyl, piperazinyl, morpholinyl and thiomorpholinyl;

Y² is selected from the group consisting of CH₂, C(O), C(S) and SO₂;

R³ is selected from the group consisting of aryl, aralkyl, C₃-C₈cycloalkyl, heteroaryl, heterocycloalkyl, C₃₋₈cycloalkyl-C₁₋₆alkyl and heterocycloalkyl-C₁₋₆alkyl; wherein the aryl, aralkyl, cycloalkyl, heteroaryl or heterocycloalkyl may be optionally substituted with one of more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino, di(C₁-C₄alkyl)amino or —(L²)ₙ—R⁴;

n is an integer selected from 0 and 1;

L² is selected from the group consisting of C₁-C₈alkyl, C₂-C₈alkenyl, C₂-C₈alkynyl, C(O), C(S), SO₂ and (A)₀₋₁—Q—(B)₀₋₁;

where A and B are each independently selected from C₁-C₆alkyl, C₂-C₆alkenyl and C₂-C₆alkynyl;

where Q is selected from the group consisting of NR⁵, O and S;

where R⁵ is selected from the group consisting of hydrogen, C₁-C₆alkyl, aryl, aralkyl, C₃₋₈cycloalkyl, heteroaryl, heterocycloalkyl, C(O)-C₁-C₆alkyl, C(O)-aryl, C(O)-aralkyl, C(O)-heteroaryl, C(O)-heterocycloalkyl, SO₂-C₁-C₆alkyl, SO₂-aryl, SO₂-aralkyl, SO₂-heteroaryl, SO₂-heterocycloalkyl and —CHR⁶R⁷;

wherein the aryl, aralkyl, cycloalkyl, heteroaryl or heterocycloalkyl may be optionally substituted with one or more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino;

where R⁶ and R⁷ are each independently selected from the group consisting of hydrogen, C₁ 6alkyl, aryl, aralkyl, C₃₋₈cycloalkyl, heteroaryl, heterocycloalkyl, C(O)—C₁₋₆alkyl, C(O)aryl, C(O)—C₃₋₈cycloalkyl, C(O)-heteroaryl and C(O)-heterocycloalkyl; wherein the aryl, aralkyl, cycloalkyl, heteroaryl or heterocycloalkyl may be optionally substituted with one or more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino;

R⁴ is selected from the group consisting of aryl, aralkyl, C₃-C₈cycloalkyl, heteroaryl and heterocycloalkyl; wherein the aryl, aralkyl, cycloalkyl, heteroaryl or heterocycloalkyl may be optionally substituted with one or more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino;

provided that when a is 0; X is CH; m is 1; L¹ is CH₂; R³ is phenyl; n is 0; and R⁴ is phenyl, wherein the phenyl group may be optionally substituted with one substituent selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino, and wherein the R⁴ group is bonded to the R³ group in the para position (i.e. when R³ and R⁴ together form biphenyl or mono-substituted biphenyl);

then R¹ and R² are each independently selected from the group consisting of hydrogen, C₂-C₆alkyl (not C₁alkyl), aryl, aralkyl, C₃-C₈cycloalkyl, C₃-C₈cycloalkyl-C₁₋₆alkyl, heteroaryl, heteroaryl-C₁₋₆alkyl, heterocycloalkyl and heterocycloalkyl-C₁₋₆alkyl; wherein the aryl, aralkyl, cycloalkyl, heteroaryl or heterocycloalkyl may be optionally substituted with one or more substituents independently selected from halogen, hydroxy, C₁-C₆alkyl, C₁-C₆alkoxy, halogenatedC₁-C₆alkyl, halogenatedC₁-C₆alkoxy, nitro, cyano, amino, C₁-C₄alkylamino, di(C₁-C₄alkyl)amino, heteroaryl or heterocycloalkyl;

alternatively, R¹ and R² may be taken together with the nitrogen atom to which they are bound to form a five to six membered monocyclic ring structure selected from the group consisting of pyrrolidinyl, piperidinyl, piperazinyl, morpholinyl and thiomorpholinyl;

provided further that when a is 0; X is N; m is 1; L¹ is CH₂; Y² is C(O)or C(S); n is 1; L² is O; R⁴ is phenyl, wherein the phenyl may be optionally substituted with one or more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino; and R¹ and R² are each independently selected from the group consisting of hydrogen and C₁₋₆alkyl;

then R³ is selected from the group consisting of aryl, aralkyl, C₃-C₈cycloalkyl, heteroaryl other than thienopyridinyl, heterocycloalkyl, C₃₋₈cycloalkyl-C₁₋₆alkyl and heterocycloalkyl-C₁₋₆alkyl; wherein the aryl, aralkyl, cycloalkyl, heteroaryl or heterocycloalkyl may be optionally substituted with one of more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino, di(C₁-C₄alkyl)amino or —(L²)ₙ—R⁴;

provided further that when a is 0; X is N; m is 1; L¹ is CH₂; Y² is C(O) or C(S); n is 0; R¹ and R² are taken together with the nitrogen to which they are bound to form pyrrolidinyl; and R⁴ is pyridyl;

then R³ is selected from the group consisting of aryl, aralkyl, C₃-C₈cycloalkyl, heteroaryl, heterocycloalkyl other than thiazolidinyl; C₃₋₈cycloalkyl-C₁₋₆alkyl and heterocycloalkyl-C₁₋₆alkyl; wherein the aryl, aralkyl, cycloalkyl, heteroaryl or heterocycloalkyl may be optionally substituted with one of more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino, di(C₁-C₄alkyl)amino or —(L²)ₙ—R⁴;

provided further that when R¹ and R² are each independently selected from the group consisting of hydrogen and C₁₋₆alkyl, or R¹ and R² are taken together with the nitrogen atom to which they are bound to form morpholinyl or pyrrolidinyl; a is 0; X is N; m is 1; L¹ is CH₂; Y² is C(O) or C(S); n is 0; and R⁴ is phenyl, wherein the phenyl is optionally substituted with one or more substituents independently selected from C₁-C₆alkyl, C₁-C₆ alkoxy, halogenatedC₁-C₆alkyl, halogenatedC₁-C₆alkoxy or nitro;

then R³ is selected from the group consisting of aryl, aralkyl, (not C₃₋₈cycloalkyl), heteroaryl, heterocycloalkyl, C₃₋₈cycloalkyl-C₁₋₆alkyl and heterocycloalkyl-C₁₋₆alkyl; wherein the aryl, aralkyl, cycloalkyl, heteroaryl or heterocycloalkyl may be optionally substituted with one substituent (not one or more) selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino;

and pharmaceutically acceptable salts thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of treating nervous system disorders in a subject in need thereof comprising administering to the subject a therapeutically effective amount of any of the compounds or pharmaceutical compositions described above.

Further illustrating the invention is a method of treating a condition selected from the group consisting of depression, schizophrenia, bipolar disorders, anxiety, emesis, acute pain, neuropathic pain, itching, migraine and movement disorders, in a subject in need thereof comprising administering to the subject a therapeutically effective amount of any of the compounds or pharmaceutical compositions described above.

In an example of the present invention is a method of treating a nervous system disorder selected from the group consisting of depression and anxiety.

Another example of the invention is the use of any of the compounds described herein in the preparation of a medicament for treating: (a) depression, (b) anxiety (c) bipolar disorder, (d) schizophrenia, (e) emesis, (f) acute pain, (g) neuropathic pain, (h) itching, (i) migraine, (j) dementia or (k) movement disorders, in a subject in need thereof.

### DETAILED DESCRIPTION OF THE INVENTION

The present invention provides novel amidoalkyl-piperidine and amidoalkyl-piperazine derivatives useful for the treatment of nervous system disorders including psychiatric disorders such as major depressive disorders with or without anxiety, anxiety disorders including generalized anxiety disorder, anticipatory anxiety in phobic (situational), anxiety as well as treatment of the anxiety component of panic disorder and obsessive-compulsive disorder, stress disorders, schizophrenic disorders and psychosis, substance abuse and withdrawal, bipolar disorder, sexual dysfunction, eating disorders; neurological disorders such as nausea and emesis: prevention and control, acute and delayed components of chemotherapy- and radiotherapy-induced emesis, drug-induced nausea and vomiting, post-operative nausea and vomiting, cyclical vomiting syndrome, psychogenic vomiting, motion sickness, sleep apnea, movement disorders such as Tourette's syndrome, cognitive disorders, as a neuroprotectant agent, cerebrovascular disease, neurodegenerative disorders (e.g. Parkinson's, ALS), pain, acute pain, eg, post-surgery, dental pain, musculoskeletal, rheumatological pain, neuropathic pain, painful peripheral neuropathy, post-herpetic neuralgia, chronic oncological- and HIV-associated pain, neurogenic, inflammatory pain, migraine; gastrointestinal disorders such as GI motility disorders, inflammatory bowel disease including both ulcerative colitis and Crohn's disease, acute diarrhea (infections, drug-induced), chronic diarrhea (inflammatory disorders eg, ulcerative colitis, HIV-associated, gastroenteritis, radiation enterocolitis; abnormal intestinal motility, eg neurological; drugs, idiopathic), irritable bowel syndrome, fecal incontinence, acute pancreatitis; urological disorders such as urinary incontinence, interstitial cystitis; dermatological disorders such as inflammatory/immunological skin disorders (eg, dermatitis herpetiform, pemphigus), atopic dermatitis, itching, urticaria and psoriasis;.

More particularly, the present invention is directed to novel amidoalkyl-piperdine and amidoalkyl-piperazine derivatives useful in the treatment of depression, dementia, schizophrenia, bipolar disorder, schizophrenia, anxiety, emesis, acute or neuropathic pain, itching, migraine and movement disorders.

Preferably, the present invention is directed to novel amidoalkyl piperidine and amidoalkyl piperazine derivatives useful in the treatment of depression or anxiety.

The compounds of the present invention were originally believed to act by modulating the neurokinin receptor, more particularly the neurokinin-1 receptor. Further testing has shown that although the compounds of the present invention may have some activity as modulators of the neurokinin-1 receptor, the activity of the compounds may also extends to modulation of other receptors and/or biological pathways, including modulation of the neurokinin-2, neurokinin-3 and the serotonin neural pathway. At this time the exact mechanism(s) of action for the compounds of the instant invention have not been determined.

The compounds of the present invention are of the formula (I):

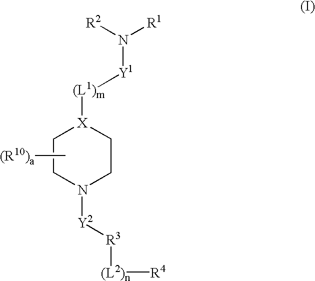

wherein a, R¹⁰, X, m, L¹, Y¹, R¹, R², Y², R³, n, L²and R⁴are as defined above.

Preferably, X is selected from the group consisting of CH, C(methyl) and N. More preferably, X is selected from the group consisting of CH and N.

Preferably, L¹ is selected from the group consisting of C₁-C₄ alkyl, more preferably L¹ is CH₂ and CH₂CH₂, most preferably L¹ is CH₂.

Preferably, Y¹ is C(O). Preferably, and Y² is C(O). More preferably Y¹ is C(O) and Y² is C(O).

Preferably, R¹ and R² are each independently selected from the group consisting of hydrogen, C₁₋₄alkyl, aryl, aralkyl, C₃₋₈cycloalkyl-C₁-C₄alkyl, heteroaryl and heterocycloalkyl; wherein the aryl, aralkyl or heteroaryl may be optionally substituted with one to two substituents independently selected from halogen, hydroxy, C₁-C₄alkyl, C₁-C₄alkoxy, trifluoromethyl, trifluoromethoxy, C₁-C₄alkylamino, di(C₁-C₄alkyl)amino or heterocycloalkyl. More preferably, R¹ is hydrogen or methyl and R² is selected from the group consisting of C₁₋₄alkyl, aryl, aralkyl, C₃₋₈cycloalkyl-C₁₋₄alkyl and heteroaryl; wherein the aryl or aralkyl may be optionally substituted with one to two substituents independently selected from halogen, hydroxy, C₁-C₄alkyl, C₁-C₄alkoxy, trifluoromethyl, trifluoromethoxy, di(C₁-C₄alkyl)amino or heterocycloalkyl. Most preferably R¹ is hydrogen and R² is selected from the group consisting of —CH₂-(3-trifluoromethylphenyl), —CH₂-cyclohexyl, —CH₂-(3,5-dimethoxyphenyl), —CH₂-(4-trifluoromethylphenyl), —CH₂-(3,5-ditrifluoromethylphenyl), 3-trifluoromethoxyphenyl, —CH₂-(4-dimethylaminophenyl), phenyl, benzyl, 2-fluorophenyl, 4-fluorophenyl, 2,4-difluorophenyl, 2,6-difluorophenyl, 4-hydroxyphenyl, 4-dimethylamino-phenyl, 3-pyridyl, 4-morpholinyl-phenyl, 4-piperidinyl-phenyl, methyl, isopropyl, 4-methoxyphenyl, 4-trifluoromethylphenyl, 2-pyrimidinyl, 4-pyrimidinyl, 2-pyridyl, 4-pyridyl, 4-pyridyl-methyl, 0.5-quinolinyl, 6-quinolinyl and 8-quinolinyl.

Alternatively, R¹ and R² may be taken together with the nitrogen atom to which they are bound to form a five to six membered monocyclic ring structure selected from the group consisting of pyrrolidinyl, piperidinyl and morpholinyl.

Preferably, R³ is selected from the group consisting of aryl and heteroaryl; wherein the aryl or heteroaryl may be optionally substituted with one to two substituents independently selected from C₁-C₄alkyl, trifluoromethyl or —(L²)ₙ—R⁴. More preferably, R³ is aryl or heteroaryl, wherein the aryl or heteroaryl may be optionally substituted with a substituent selected from C₁-C₄alkyl or trifluoromethyl. Most preferably, R³ is selected from the group consisting of phenyl, methylphenyl, trifluoromethylphenyl, 4-oxazolyl and 3-(2-.trifluoromethyl-furyl).

Preferably, L² is selected from the group consisting of C₁-C₆alkyl, C₂-C₆alkenyl, C₂-C₆alkynyl and (A)₀₋₁—Q—(B)₀₋₁;

where A and B are each independently selected from C₁-C₄alkyl;

where Q is selected from the group consisting of NR⁵, O and S;

where R⁵ is selected from the group consisting of hydrogen, C₁-C₄alkyl, C(O)-C₁-C₆alkyl, C(O)-aryl, C(O)-aralkyl, C(O)-heteroaryl, C(O)-heterocycloalkyl and —CHR⁶R⁷; wherein the aryl, aralkyl, cycloalkyl, heteroaryl or heterocycloalkyl may be optionally substituted with one to two substituents independently selected from halogen, C₁-C₄alkyl, C₁-C₄alkoxy, trifluoromethyl, trifluoromethoxy, nitro, cyano, amino, C₁-C₄alkylamino or di(C₁-C₄alkyl)amino;

where R⁶ and R⁷ are each independently selected from the group consisting of hydrogen, C₁₋₄alkyl, aryl, aralkyl, C₃₋₈cycloalkyl, heteroaryl, heterocycloalkyl, C(O)-C₁₋₆alkyl, C(O)aryl, C(O)-C₃₋₈cycloalkyl, C(O)-heteroaryl and C(O)-heterocycloalkyl; wherein the aryl, aralkyl, cycloalkyl, heteroaryl or heterocycloalkyl may be optionally substituted with one to two substituents independently selected from halogen, hydroxy, C₁-C₄alkyl, C₁-C₄ alkoxy, trifluoromethyl, trifluoromethoxy, nitro, cyano, amino, C₁-C₄alkylamino or di(C₁-C₄alkyl)amino.

More preferably, L² is selected from the group consisting of C₁-C₄alkyl, C₂-C₆alkenyl, C₂-C₆alkynyl, NH—C₁₋₄alkyl, C₁₋₄alkyl-N(C₁₋₄alkyl)-C₁₋₄alkyl and C₁₋₄alkyl-N(C(O)C₁₋₄alkyl)-C₁₋₄alkyl. In a further class of the invention, L² is selected from the group consisting of

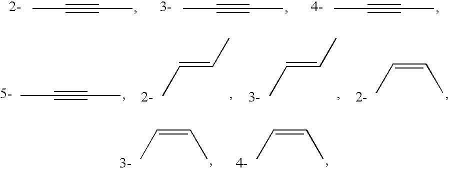

2—CH₂CH₂, 3—CH₂—CH₂, 4—CH₂—CH₂, NH—CH₂, CH₂—N(CH₃)-CH₂, CH₂—N(CH₃)—CH₂CH₂, CH₂—N(C(O)CH₃)—CH₂ and CH₂—N(C(O)CH₃)—CH₂CH₂.

Preferably, R⁴ is selected from the group consisting of aryl, heteroaryl and heterocycloalkyl; wherein the aryl group may be optionally substituted with one to two substituents independently selected from hydroxy, halogen, C₁-C₄alkyl, C₁₋₄alkoxy, trifluoromethyl or amino. More preferably, R⁴ is selected from the group consisting of phenyl, 2-pyridyl, 3-pyridyl, 4-pyridyl, 3-hydroxyphenyl, 2-methylphenyl, 3-aminophenyl, 3-thienyl, 3,5-di(trifluoromethyl )-phenyl, 4-methoxyphenyl, 4-chlorophenyl, 2-thienyl, 2-furyl, 1-pyrrolidinyl, 1-imidazolyl, 2-benzimidazolyl, naphthyl and tetrahydrofuryl.

In a class of the invention a is an integer selected from 0 and 1. In a preferred embodiment, a is 0 such that R¹⁰ is absent. However, in a subclass of the invention, a is 1. In that instance, R¹⁰ is preferably selected from the group consisting of C₁-C₄alkyl and aralkyl; more preferably, R¹⁰ is selected from the group consisting of methyl and benzyl.

In another class of the present invention is a compound of formula (I) wherein a is 0; X is selected from the group consisting of CH and N; Y¹ is C(O); m is 1; L¹ is CH₂; R¹ is hydrogen; R² is selected from the group consisting of phenyl, 4-hydroxyphenyl, 2-fluorophenyl, 4-fluorophenyl, and 2,4-difluorophenyl; Y² is C(O); R³ is phenyl; n is 1; L² is selected from the group consisting of

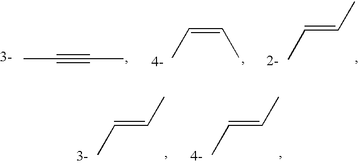

4—(CH₂—N(CH₃)—CH₂CH₂), 4—(CH₂—N(CH₃)—CH₂) and 3—NH—CH₂; R⁴ is selected from the group consisting of 2-pyridyl, 4-pyridyl, 4-pyrrolidinyl, 2-furyl, 1-naphthyl and 3,5-di(trifluoromethyl)phenyl; and pharmaceutically acceptable salts thereof.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following:

acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methyinitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

As used herein, “halogen” shall mean chlorine, bromine, fluorine and iodine.

As used herein, the term “alkyl” whether used alone or as part of a substituent group, include straight and branched chains comprising one to ten carbon atoms. For example, alkyl radicals include methyl, ethyl, propyl, isopropyl, butyl, isobutyl, sec-butyl, t-butyl, pentyl and the like. Unless otherwise noted, “lower” when used with alkyl means a carbon chain composition of one to six carbon atoms.

The term “alkenyl”, whether used alone or as part of a substituent group, shall include straight and branched alkene chains comprising two to ten carbon atoms. Suitable examples include vinyl, 1-propenyl, 2-propenyl, 1-butenyl. 2-butenyl, 1-pentenyl, 2-pentenyl, 1-isobut-2-enyl, and the like.

The term “alkynyl”, whether used alone or as part of a substituent group, shall include straight and branched alkyne chains comprising two to ten carbon atoms. Suitable examples include 2-propynyl, 2-butynyl, 1-butynyl, 1-pentynyl, and the like.

The term “proximal alkenyl” and “proximal alkynyl” when used in conjunction with L², shall denote an alkenyl or alkynyl chain, where the terminal carbon atom is partially unsaturated. Suitable example include

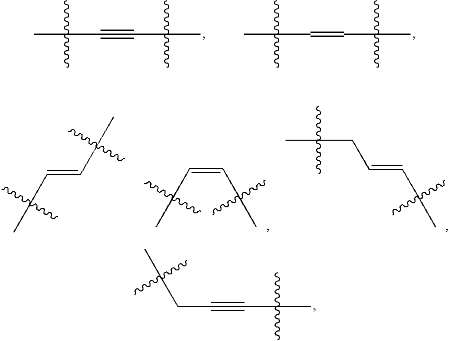

and the like.

As used herein, unless otherwise noted, “alkoxy” shall denote an oxygen ether radical of the above described straight or branched chain alkyl groups. For example, methoxy, ethoxy, n-propoxy, sec-butoxy, t-butoxy, n-hexyloxy and the like.

As used herein, unless otherwise noted, “cycloalkyl” shall refer to a monocyclic, saturated ring structure comprising three to eight carbon atoms. Suitable examples include cyclopropyl, cyclobutyl, cyclopentyl, cyclohexyl, cycloheptyl and cylclooctyl.

As used herein, unless otherwise noted, “aryl” shall refer to carbocyclic aromatic groups such as phenyl, naphthyl, and the like.

As used herein, unless otherwise noted, “aralkyl” shall mean any lower alkyl group substituted with an aryl group such as phenyl, naphthyl and the like. For example, benzyl, phenylethyl, phenylpropyl, naphthylmethyl, and the like.

As used herein, unless otherwise noted, “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to three additional heteroatoms independently selected from the group consisting of O, N and S. The heteroaryl group may be attached at any heteroatom or carbon atom of the ring such that the result is a stable structure.

Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, isoxazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like. Preferred heteroaryl groups include pyridyl, thienyl, furyl, imidazolyl, indolyl, oxazolyl, isoxazolyl, pyrimidinyl, quinolinyl and benzimidazolyl.

As used herein, the term “heterocycloalkyl” shall denote any five to seven membered monocyclic, saturated, partially unsaturated or partially aromatic ring structure containing at least one heteroatom selected from the group consisting of O, N and S, optionally containing one to three additional heteroatoms independently selected from the group consisting of O, N and S; or a nine to ten membered saturated, partially unsaturated or partially aromatic bicyclic ring system containing at least one heteroatom selected from the group consisting of O, N and S, optionally containing one to three additional heteroatoms independently selected from the group consisting of O, N and S. The heterocycloalkyl group may be attached at any heteroatom or carbon atom of the ring such that the result is a stable structure.

Examples of suitable heterocycloalkyl groups include, but are not limited to, pyrrolinyl, pyrrolidinyl, dioxalanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 3,4-methylenedioxyphenyl, 2,3-dihydrobenzofuryl, isoxazolinyl, tetrahydrofuryl, and the like. Preferred heterocycloalkyl groups include tetrahydrofuryl, pyrrolidinyl, piperidinyl, piperazinyl, morpholinyl, pyrazolidinyl and isoxazolinyl.

As used herein, the notation “*” shall denote the presence of a stereogenic center.

When a particular group is “substituted” (e.g., aryl, cycloalkyl, heteroaryl, heterocycloalkyl), that group may have one or more substituents, preferably from one to five substituents, more preferably from one to three substituents, most preferably from one to two substituents, independently selected from the list of substituents.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

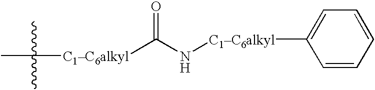

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As used herein, unless otherwise noted, the term “nervous system disorder” shall include major depressive disorders with or without anxiety, anxiety disorders, generalized anxiety disorder, anticipatory anxiety in phobic (situational), the anxiety component of panic disorder, the anxiety component of obsessive-compulsive disorder, stress disorder, schizophrenic disorders, psychosis, substance abuse and withdrawal, bipolar disorder, sexual dysfunction, eating disorders; nausea, emesis (including both prevention and control), acute chemotherapy- and radiotherapy-induced emesis, delayed chemotherapy- and radiotherapy-induced emesis, drug-induced nausea and vomiting, post-operative nausea and vomiting, cyclical vomiting syndrome, psychogenic vomiting, motion sickness, sleep apnea, Tourefte's syndrome, cognitive disorders, cerebrovascular disease, neurodegenerative disorders, Alzheimer's disease, Parkinson's disease, amyotrophic lateral sclerosis (ALS) pain, acute pain, post-surgical pain, dental pain, musculoskeletal, rheumatological pain, neuropathic pain, painful peripheral neuropathy, post-herpetic neuralgia, chronic oncological pain, HIV-associated pain, neurogenic, inflammatory pain, migraine; GI motility disorders, inflammatory bowel disease, ulcerative colitis, Crohn's disease, acute diarrhea (infection and drug-induced), chronic diarrhea, gastroenteritis, radiation enterocolitis; abnormal intestinal motility, irritable bowel syndrome, fecal incontinence, acute pancreatitis; urinary incontinence, interstitial cystitis; i dermatitis herpetiform, pemphigus, atopic dermatitis, itching, urticaria and psoriasis;.

Preferred nervous system disorders include depression, anxiety, bipolar disorder, schizophrenia, emesis, migraine, itching, acute pain, neuropathic pain and movement disorders. Most preferred nervous system disorders include depression and anxiety.

Abbreviations used in the specification, particularly the Schemes and Examples, are as follows:

| | |
|---|---|
| BOC or Boc = | t-butoxycarbonyl |
| BSA = | bovine serum albumin |
| DCE = | dichloroethane |
| DCM = | dichloromethane |
| DEA = | diethylamine |
| DIC = | diisopropylcarbodiimide |
| DIPEA = | diisopropylethylamine |
| DMAP = | 4-N,N-dimethylaminopyridine |
| DME = | 1,2-dimethoxyethane |
| DMF = | dimethyl formamide |
| Et = | ethyl |
| EtOAc = | ethyl acetate |
| EtOH = | ethanol |
| Et₂O = | diethyl ether |
| Fmoc = | 9H-fluoren-9-ylmethoxycarbonyl |
| FMPB = | 4-(4-formyl-3-methoxyphenoxy)butyryl AM |
| | resin |
| HEPES = | 4-(2-Hydroxyethyl)-1-piperizine ethane |
| | sulfonic acid |
| HATU = | O-(7-Azabenzotriazol-1-yl)-N,N,N″,N″- |
| | Tetramethyl Uronium Hexafluorophosphate |
| HOAT = | 1-hydroxy-7-azabenzotriazole |
| HOBT = | 1-Hydroxybenzotriazole |
| Me = | methyl |
| NaBH(OAc)₃ = | sodium triacetoxyborohydride |
| NMP = | N-Methyl-2-pyrrolidinone |
| Ph = | phenyl |
| RT or rt = | room temperature |
| TEA = | triethylamine |
| TFA = | trifluoroacetic acid |
| THF = | tetrahydrofuran |
| TMOF = | trimethylorthoformate |

The compounds of the instant invention may be prepared according to the processes outlined in Scheme 1 through 21.

Compounds of formula (I) wherein X is CH, m is 1, L¹ is CH₂, Y¹ is C(O), Y² is C(O), n is 1 and L² is a proximal alkenyl or proximal alkynyl, may be prepared according to the process outlined in Scheme 1.

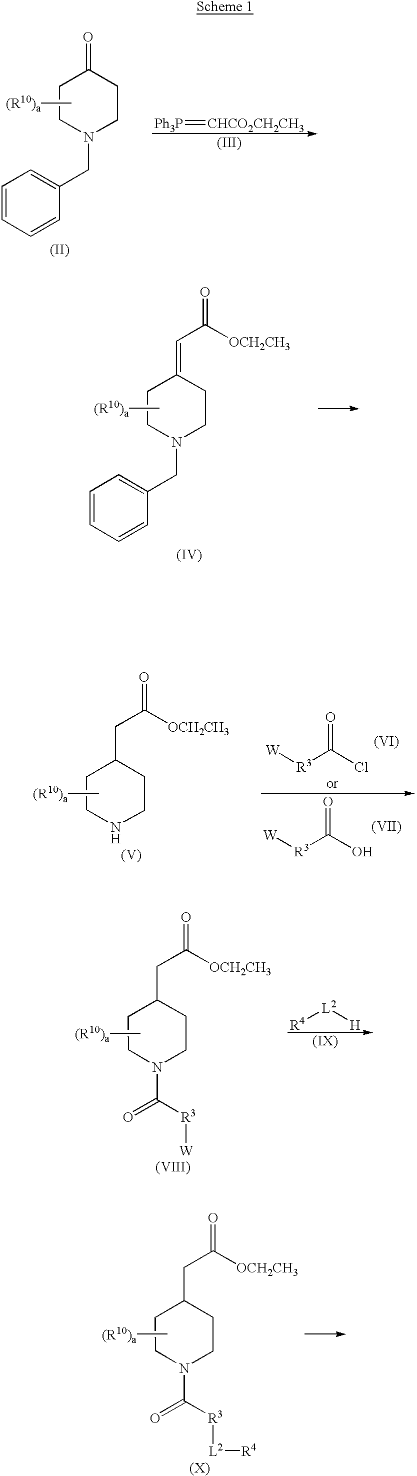

More specifically, a suitably substituted compound of formula (II), a known compound or compound prepared by known methods, is reacted with a Wittig reagent, such as (carbethoxymethylene) triphenylphosphorane, a compound of formula (III), in the presence of a hydrocarbon solvent such as toluene, benzene, xylene, and the like, at an elevated temperature, preferably at about reflux temperature, to yield the corresponding compound of formula (IV).

The compound of formula (IV) is de-protected and reduced by treating with hydrogen gas at an elevated pressure in the range of about 45-50 psig, in the presence of a solvent such as ethanol, methanol, and the like, in the presence of a catalyst such as Pearlman's catalyst, and the like, to yield the corresponding compound of formula (V).

The compound of formula (V) is reacted with a suitably substituted acid chloride of formula (VI), wherein W is iodine or bromine, in the presence of an organic base such as triethylamine, diisopropylethylamine, and the like, in a halogenated solvent such as methylene chloride, chloroform, and the like, at a temperature from about 0° C. to room temperature, to yield the corresponding compound of formula (VIII).

Alternatively, the compound of formula (V) is reacted with a suitably substituted carboxylic acid of formula (VII), wherein W is iodine or bromine, in the presence of a coupling agent such as HATU, in the presence of a coupling additive such as HOBT, in the presence of an organic base such as TEA, DIPEA, and the like, in an organic solvent such as DMF, methylene chloride, chloroform, and the like, to yield the corresponding compound of formula (VIII).

The compound of formula (VIII) is reacted with a compound of formula (IX), wherein L² is a proximal alkenyl or proximal alkynyl, such as

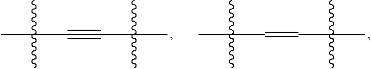

and the like, in the presence of a copper salt such as copper(I)iodide, and the like, in the presence of a palladium catalyst such as palladium (II) chloride, palladium acetate, and the like, in the presence of an organic base such as TEA, DEA, and the like, in an organic solvent such as DMF, and the like, at an elevated temperature, preferably at a temperature in the range of about 80-130° C., in a sealed tube, to yield the corresponding compound of formula (X).

The compound of formula (X) is reacted with an aqueous base such as lithium hydroxide, sodium hydroxide, potassium carbonate, and the like, in an ethereal solvent such as THF, dioxane, and the like, to yield the corresponding compound of formula (XI).

The compound of formula (XI) is coupled to a suitably substituted amine, a compound of formula (XII), in the presence of a coupling agent such as isobutylchloroformate, HATU, and the like, in the presence of an organic base such as TEA, DIPEA, and the like, in a halogenated solvent such as methylene chloride, chloroform, and the like, at about 0° C. to about ambient temperature, to produce the corresponding compound of formula (Ia).

When the compound of formula (XII) is a secondary amine, the coupling agent is preferably HATU. When the compound of formula (XII) is a cyclic secondary amine (e.g. pyrrolidine, piperidine, morpholine, and the like), the coupling agent is preferably HATU and further preferably is in the presence of a coupling additive such as HOBT, and the like.

Compounds of formula (I) wherein X is N, m is 1, L¹ is CH₂, Y¹ is C(O), Y² is C(O), n is 1 and L² is a proximal alkenyl or proximal alkynyl may be prepared according to the process outlined in Scheme 2.

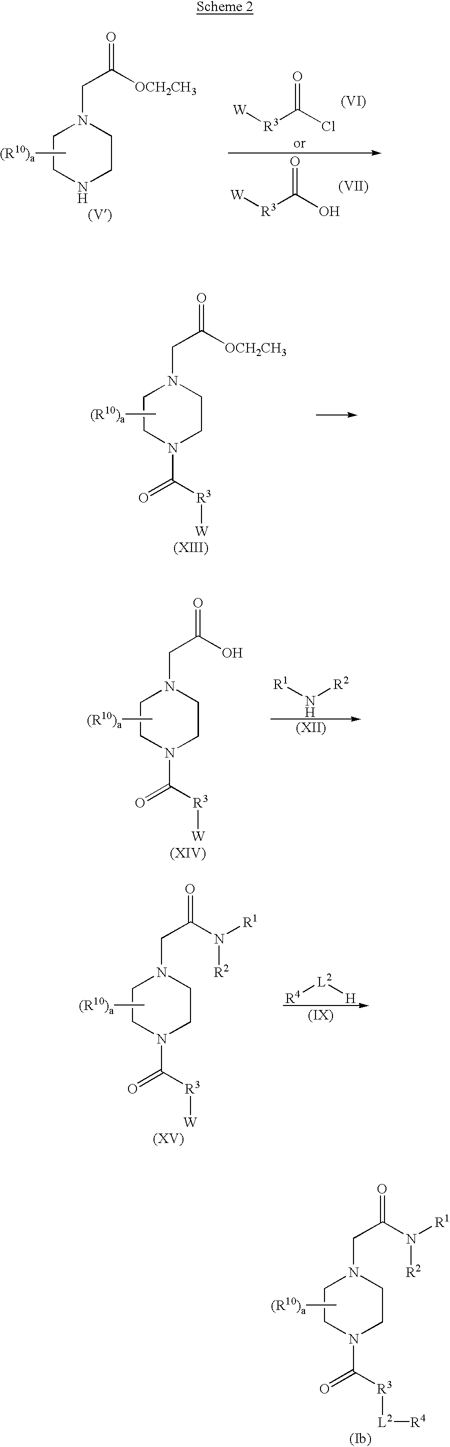

More specifically, a suitably substituted compound of formula (V′), a known compound (available from Lancaster) is reacted with a suitably substituted acid chloride of formula (VI), wherein W is iodine or bromine, in the presence of an organic base such as TEA, DIPEA, and the like, in a halogenated solvent such as methylene chloride, chloroform, and the like, at a temperature from about 0° C. to room temperature, to yield the corresponding compound of formula (XIII).

Alternatively, a suitably substituted compound of formula (V) is reacted with a suitably substituted carboxylic acid of formula (VII), wherein W is iodine or bromine, in the presence of a coupling agent such as HATU, in the presence of a coupling additive such as HOBT, in the presence of an organic base such as TEA, DIPEA, and the like, in an organic solvent such as DMF, methylene chloride, chloroform, and the like, to yield the corresponding compound of formula (XIII).

The compound of formula (XIII) is reacted with an aqueous base such as lithium hydroxide, sodium hydroxide, potassium carbonate, and the like, in an ethereal solvent such as THF, dioxane, and the like, to yield the corresponding compound of formula (XIV).

The compound of formula (XIV) is coupled to a suitably substituted amine, a compound of formula (XII), in the presence of a coupling agent such as isobutylchloroformate, HATU, and the like, in the presence of an organic base such as TEA, DI PEA, and the like, in a halogenated solvent such as methylene chloride, chloroform, and the like, at about 0° C. to about ambient temperature, to produce the corresponding compound of formula (XV).

When the compound of formula (XII) is a secondary amine, the coupling agent is preferably HATU. When the compound of formula (XII) is a cyclic secondary amine, the coupling agent is preferably HATU and further preferably is in the presence of a coupling additive such as HOBT, and the like.

The compound of formula (XV) is reacted with a compound of formula (IX), wherein L² is a proximal alkenyl or proximal alkynyl such as

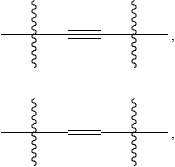

and the like, in the presence of a copper salt such as copper(I)iodide, and the like, in the presence of a palladium catalyst such as palladium (II) chloride, palladium acetate, Pd(PPh₃)₄, and the like, in the presence of an organic base such as TEA, DEA, and the like, in an organic solvent such as DMF, and the like, at an elevated temperature, preferably at a temperature in the range of about 80-130° C., in a sealed tube, to yield the corresponding compound of formula (Ib).

Compounds of formula (I) wherein m is 1, L¹ is CH₂, Y¹ is C(O), Y₂ is SO₂, n is land L² is a proximal alkenyl or proximal alkynyl may be prepared according to the process outlined in Scheme 3.

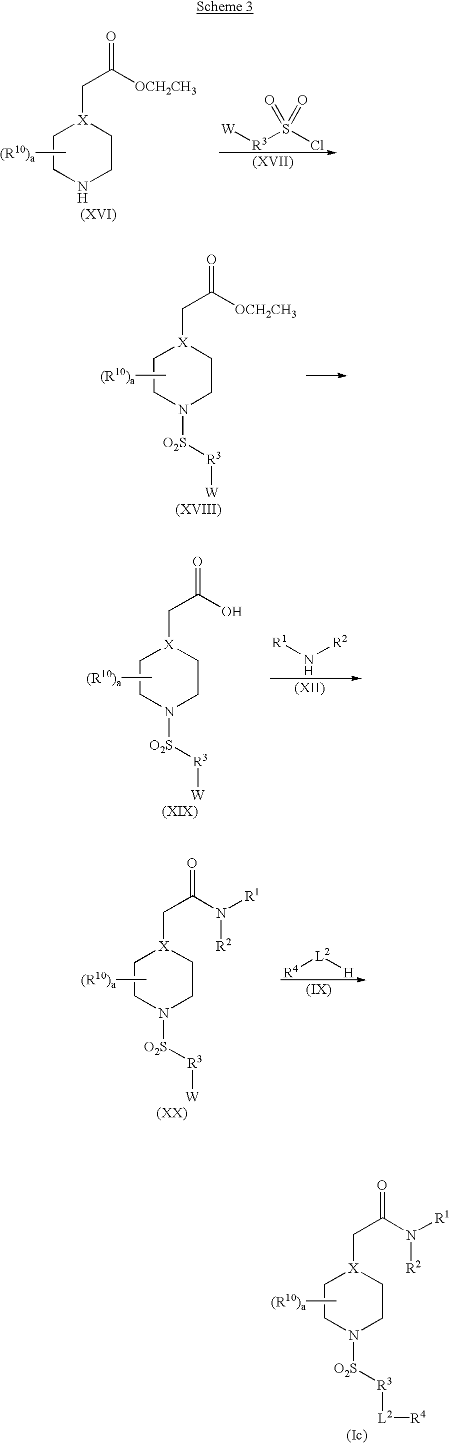

More specifically, a compound of formula (XVI), a known compound or compound prepared by known methods is reacted with a suitably substituted sulfonyl chloride, a compound of formula (XVII), wherein W is iodine or bromine, in the presence of an organic base such as TEA, DIPEA, and the like, in a halogenated solvent such as methylene chloride, chloroform, and the like, with heating from a temperature of about 0° C. to room temperature, to yield the corresponding compound of formula (XVIII).

The compound of formula (XVIII) is reacted with an aqueous base such as lithium hydroxide, sodium hydroxide, potassium carbonate, and the like, in an ethereal solvent such as THF, and the like, to yield the corresponding compound of formula (XIX).

The compound of formula (XIX) is coupled to a suitably substituted amine, a compound of formula (XII), in the presence of a coupling agent such as isobutylchloroformate, HATU, and the like, in the presence of an organic base such as TEA, DIPEA, and the like, in a halogenated solvent such as methylene chloride, chloroform, and the like, at about 0° C. to about ambient temperature, to produce the corresponding compound of formula (XX).

When the compound of formula (XII) is a secondary amine, the coupling agent is preferably HATU. When the compound of formula (XII) is a cyclic secondary amine, the coupling agent is preferably HATU and further preferably is in the presence of a coupling additive such as HOBT, and the like.

The compound of formula (XX) is reacted with a compound of formula (IX), wherein L² is a proximal alkenyl or proximal alkynyl, such as

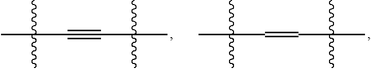

and the like, in the presence of a copper salt such as copper(I)iodide, and the like, in the presence of a palladium catalyst such as palladium (II) chloride, palladium acetate, Pd(PPh₃)₄, and the like, in the presence of an organic base such as TEA, DEA, and the like, in an organic solvent such as DMF, and the like, at an elevated temperature, preferably at a temperature in the range of about 80-130° C., in a sealed tube, to yield the corresponding compound of formula (Ic).

Compounds of formula (I) wherein X is C(C₁-C₆alkyl), m is 1, L¹ is CH₂, Y¹ is C(O) and Y² is C(O) can be prepared according to the process outlined in Scheme 4.

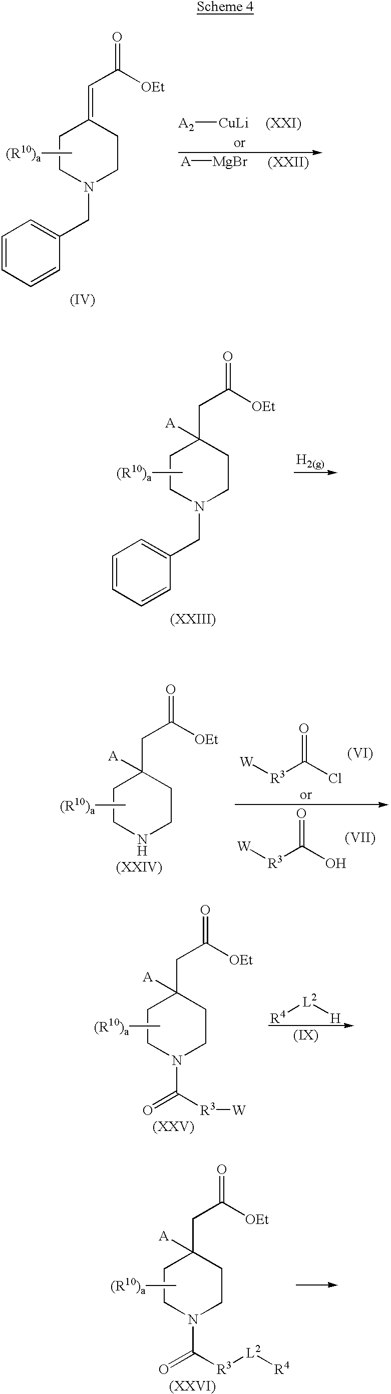

Accordingly, a compound of formula (IV), prepared as in Scheme 1, is coupled via a 1,4-conjugate addition reaction with a suitably substituted lithium dialkyl copper reagent, a compound of formula (XXI), wherein A is C₁-C₆alkyl, such as lithium dimethyl cuprate, lithium diethyl cuprate, and the like, in the presence of an ethereal solvent such as THF, ethyl ether, and the like, optionally in the presence of a Lewis acid such as BF₃, and the like, to yield the corresponding compound of formula (XXIII).

Alternatively, the compound of formula (IV) may be coupled via a 1,4-conjugate addition using a Grignard reagent, a compound of formula (XXII), wherein A is C₁-C₆alkyl, such as methyl magnesium bromide, ethyl magnesium bromide, and the like, in the presence of a copper catalyst such as CuCl, and the like, in the presence of an ethereal solvent such as diethyl ether, THF, and the like, to yield the corresponding compound of formula (XXIII).

The compound of formula (XXIII) is de-protected and reduced by treating with hydrogen gas at an elevated pressure in the range of about 45-50 psig, in the presence of a solvent such as ethanol, methanol, and the like, in the presence of a catalyst such as Pearlman's catalyst, and the like, to yield the corresponding compound of formula (XXIV).

The compound of formula (XXIV) is reacted with a suitably substituted acid chloride of formula (VI), wherein W is iodine or bromine, in the presence of an organic base such as TEA, DIPEA, and the like, in a halogenated solvent such as methylene chloride, chloroform, and the like, at about 0° C. to room temperature, to yield the corresponding compound of formula (XXV).

Alternatively, the compound of formula (XXIV) is reacted with a suitably substituted carboxylic acid of formula (VII), wherein W is iodine or bromine, in the presence of a coupling agent such as HATU, in the presence of a coupling additive such as HOBT, in the presence of an organic base such as TEA, DIPEA, and the like, in an organic solvent such as DMF, methylene chloride, chloroform, and the like, to yield the corresponding compound of formula (XXV).

The compound of formula (XXV) is reacted with a compound of formula (IX), wherein L² is a proximal alkenyl or proximal alkynyl, such as

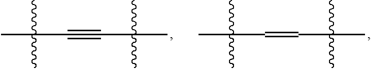

and the like, in the presence of a copper salt such as copper(I)iodide, and the like, in the presence of a palladium catalyst such as palladium (II) chloride, palladium acetate, Pd(PPh₃)₄, and the like, in the presence of an organic base such as TEA, DEA, and the like, in an organic solvent such as DMF, and the like, at an elevated temperature, preferably at a temperature in the range of about 80-130° C., in a sealed tube, to yield the corresponding compound of formula (XXVI).

The compound of formula (XXVI) is reacted with an aqueous base such as lithium hydroxide, sodium hydroxide, potassium carbonate, and the like, in an ethereal solvent such as THF, dioxane, and the like, to yield the corresponding compound of formula (XXVII).

The compound of formula (XXVII) is coupled to a suitably substituted amine, a compound of formula (XII), in the presence of a coupling agent such as isobutylchloroformate, HATU, and the like, in the presence of an organic base such as TEA, DIPEA, and the like, in a halogenated solvent such as methylene chloride, chloroform, and the like, at about 0° C. to about ambient temperature, to produce the corresponding compound of formula (Id).

When the compound of formula (XII) is a secondary amine, the coupling agent is preferably HATU. When the compound of formula (XII) is a cyclic secondary amine, the coupling agent is preferably HATU and further preferably is in the presence of a coupling additive such as HOBT, and the like.

Compounds of formula (I) wherein m is 1, L¹ is (CH₂)₀₋₆, Y¹ is C(O) and Y² is C(O) may be prepared according to the process outlined in Scheme 5.

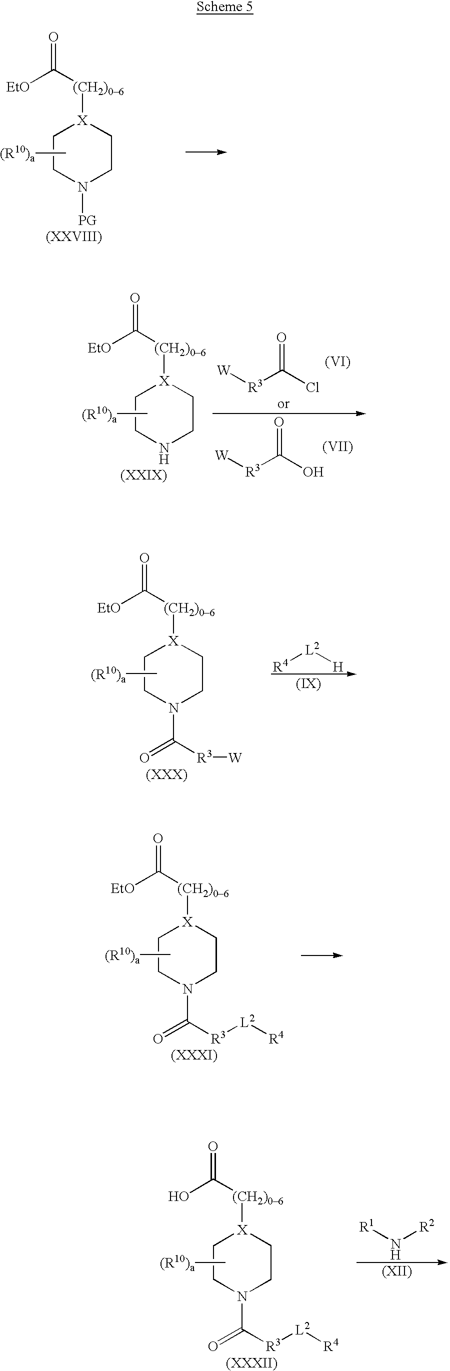

Accordingly, a compound of formula (XXVIII), a known compound or compound prepared by known methods, wherein PG is a protecting group such as BOC, benzyl, Fmoc, and the like, is de-protected by known methods (for example if the protecting group is an acid labile group, such as BOC, and the like, the de-protection is effected by treating with an acid such as TFA, HCl, and the like; if the protecting group is benzyl group, the de-protection is effected by treating with hydrogen gas at a pressure in the range of about 45-50 psig, in the presence of a solvent such as ethanol, methanol, and the like, in the presence of a catalyst such as Pearlman's catalyst, and the like), to yield the corresponding compound of formula (XXIX).

The compound of formula (XXIX) is reacted with a suitably substituted acid chloride of formula (VI), wherein W is iodine or bromine, in the presence of an organic base such as TEA, DIPEA, and the like, in a halogenated solvent such as methylene chloride, chloroform, and the like, at a temperature from about 0° C. to room temperature, to yield the corresponding compound of formula (XXX).

Alternatively, the compound of formula (XXIX) is reacted with a suitably substituted carboxylic acid of formula (VII), wherein W is iodine or bromine, in the presence of a coupling agent such as HATU, in the presence of a coupling additive such as HOBT, in the presence of an organic base such as TEA, DIPEA, and the like, in an organic solvent such as DMF, methylene chloride, chloroform, and the like, to yield the corresponding compound of formula (XXX).

The compound of formula (XXX) is reacted with a compound of formula (IX), wherein L² is a proximal alkenyl or proximal alkynyl, such as

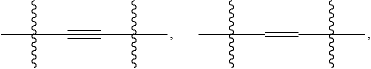

and the like, in the presence of a copper salt such as copper(I)iodide, and the like, in the presence of a palladium catalyst such as palladium (II) chloride, palladium acetate, Pd(PPh₃)₄, and the like, in the presence of an organic base such as TEA, DEA, and the like, in an organic solvent such as DMF, and the like, at an elevated temperature, preferably at a temperature in the range of about 80-130° C., in a sealed tube, to yield the corresponding compound of formula (XXXI).

The compound of formula (XXXI) is reacted with an aqueous base such as lithium hydroxide, sodium hydroxide, potassium carbonate, and the like, in an ethereal solvent such as THF, dioxane, and the like, to yield the corresponding compound of formula (XXXII).

The compound of formula (XXXII) is coupled to a suitably substituted amine, a compound of formula (XII), in the presence of a coupling agent such as isobutylchloroformate, HATU, and the like, in the presence of an organic base such as TEA, DIPEA, and the like, in a halogenated solvent such as methylene chloride, chloroform, and the like, at about 0° C. to about ambient temperature, to produce the corresponding compound of formula (Ie).

When the compound of formula (XII) is a secondary amine, the coupling agent is preferably HATU. When the compound of formula (XII) is a cyclic secondary amine, the coupling agent is preferably HATU and further preferably is in the presence of a coupling additive such as HOBT, and the like.

Compounds of formula (XXVIII) wherein L¹ is (CH₂)₄₋₆ and PG is benzyl may be prepared according to the process outlined in Scheme 6.

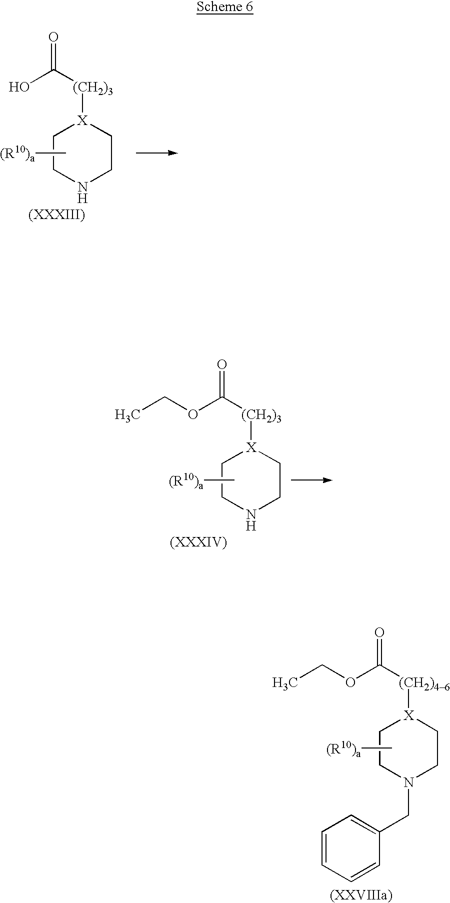

More particularly, a compound of formula (XXXIII), a known compound, is reacting with an alcohol such as methanol, ethanol, and the like, in the presence of an acid such as TFA, HCl, and the like, followed by protection of the amine group by reacting with benzylhalide, in the presence of a base such as TEA, pyridine, and the like, in an organic solvent such as DMF, THF, and the like, to yield the corresponding compound of formula (XXXIV).

The compound of formula (XXXIV) is subjected to sequential homologation by reacting the compound of formula (XXXIV) with Br₂CHLi, followed by reacting with butyl lithium, preferably at a temperature in the range of room temperature to about 100° C., to yield the corresponding compound of formula (XXVIIIa). For compounds of formula (XXVIIIa) wherein L is (CH₂)₄, the homologation is performed once, for compounds of formula (XXVIIIa) wherein L is (CH₂)₅, homologation is performed two times, for compounds of formula (XXVIIIa) wherein L is (CH₂)₆, homologation is performed three times.

Compounds of formula 1 wherein n is 0 (i.e. L² is absent) and Y² is C(O) or SO₂ may be prepared according to the process outlined in Scheme 7.

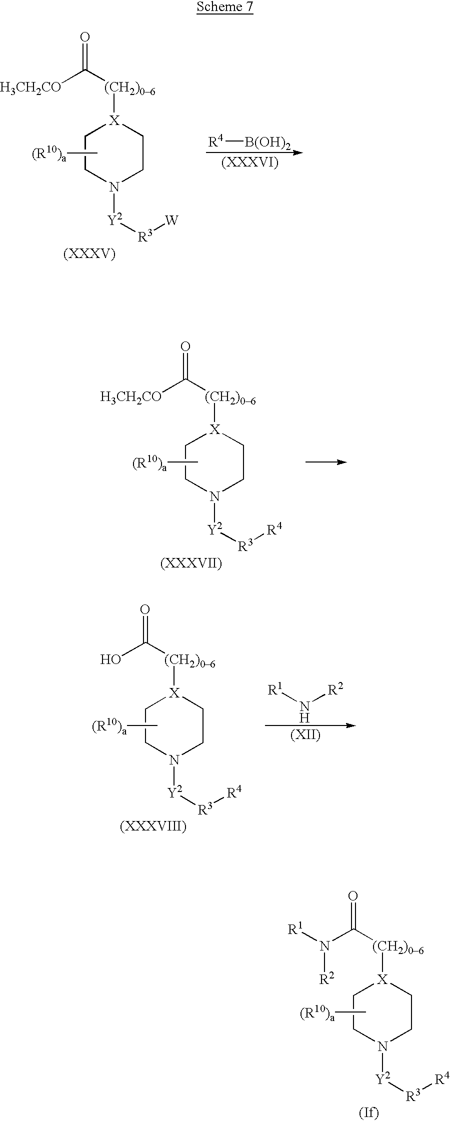

More particularly, a compound of formula (XXXV), a known compound or compound prepared by known methods, is reacted with a suitably substituted compound of formula (XXXVI), in the presence of a palladium catalyst such as tetrakistriphenylphosphine palladium(0), bis(triphenylphosphine)palladium(II) chloride, palladium acetate, and the like, in the presence of a base such as sodium carbonate, cesium carbonate, and the like, in an organic alcohol such as ethanol, methanol, and the like, in an organic solvent such as toluene, xylene, and the like, at a temperature in the range of about ambient to reflux, to yield the corresponding compound of formula (XXXVII).

The compound of formula (XXXVII) is hydrolyzed by reacting with an aqueous solution of a base such as LiOH, NaOH, K₂CO₃, and the like, in an ethereal solvent such as THF, dioxane, and the like, to yield the corresponding compound of formula (XXXVIII).

The compound of formula (XXXVIII) is coupled to a suitably substituted amine, a compound of formula (XII), in the presence of a coupling agent such as isobutylchloroformate, HATU, and the like, in the presence of an organic base such as TEA, DIPEA, and the like, in a halogenated solvent such as methylene chloride, chloroform, and the like, at about 0° C. to about ambient temperature, to produce the corresponding compound of formula (If).

When the compound of formula (XII) is a secondary amine, the coupling agent is preferably HATU. When the compound of formula (XII) is a cyclic secondary amine, the coupling agent is preferably HATU and further preferably is in the presence of a coupling additive such as HOBT, and the like.

Compounds of formula (I) wherein Y² is CH₂ or C(S) may be prepared according to the process outline in Scheme 8.

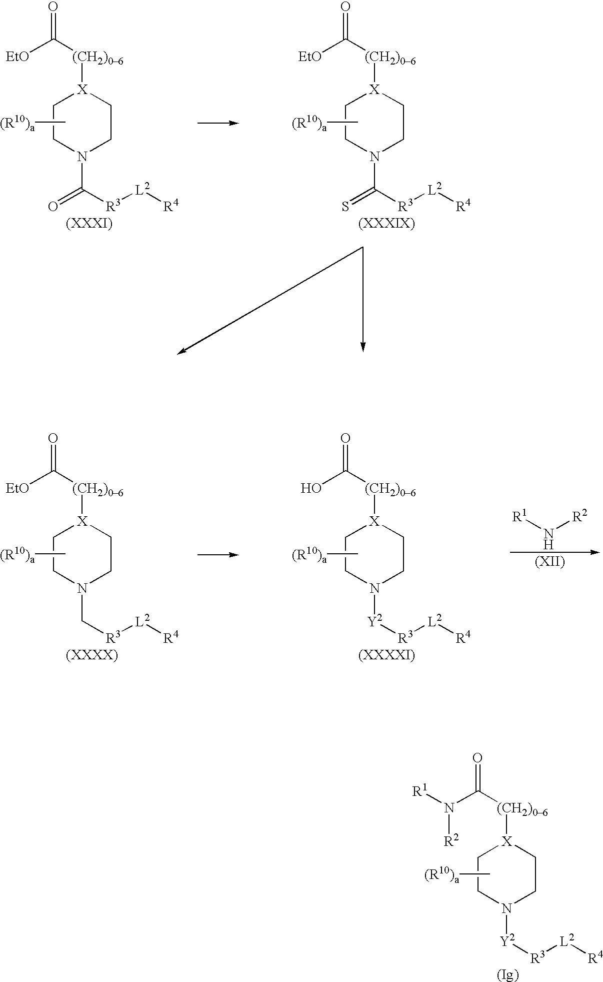

Accordingly, a compound of formula (XXXI), prepared as in Scheme 5, is reacted with Lawesson's reagent, to yield the corresponding compound of formula (XXXIX).

The compound of formula (XXXIX) is reduced in the presence of a nickel catalyst such as Raney nickel, nickel boride, and the like, in the presence of an ethereal solvent such as THF, methanol, ethanol, and the like, to yield the corresponding compound of formula (XXXX).

The compound of formula (XXXX) is hydrolyzed by reacting with an aqueous solution of a base such as LiOH, NaOH, K₂CO₃, and the like, in an ethereal solvent such as THF, dioxane, and the like, to yield the corresponding compound of formula (XXXXI), wherein Y² is CH₂.

Alternatively, the compound of formula (XXXIX) is directly hydrolyzed by reacting with an aqueous solution of a base such as LiOH, NaOH, K₂CO₃, and the like, in an ethereal solvent such as THF, dioxane, and the like, to yield the corresponding compound of formula (XXXXI), wherein Y² is C(S).

The compound of formula (XXXXI) is coupled to a suitably substituted amine, a compound of formula (XII), in the presence of a coupling agent such as isobutylchloroformate, HATU, and the like, in the presence of an organic base such as TEA, DIPEA, and the like, in a halogenated solvent such as methylene chloride, chloroform, and the like, at about 0° C. to about ambient temperature, to produce the corresponding compound of formula (Ig).

When the compound of formula (XII) is a secondary amine, the coupling agent is preferably HATU. When the compound of formula (XII) is a cyclic secondary amine, the coupling agent is preferably HATU and further preferably is in the presence of a coupling additive such as HOBT, and the like.

Compounds of formula (I) wherein L² is C₂-C₈alkyl may be prepared according to the process outlined in Scheme 9.

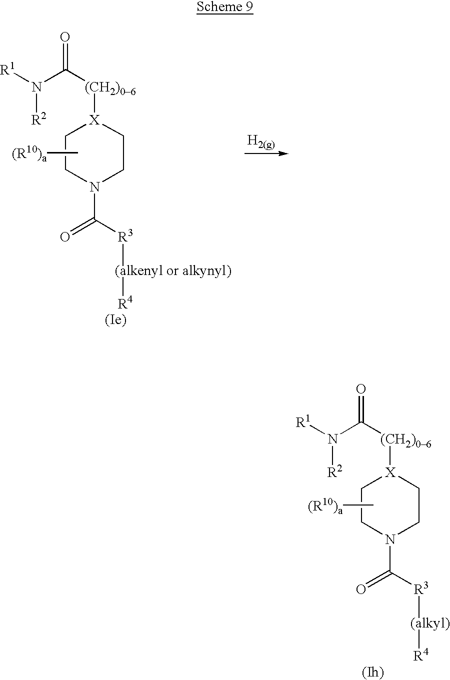

More particularly, a compound of formula (le), wherein L² is C₂-C₈alkenyl or C₂-C₈alkynyl, prepared as in Scheme 5, is reduced by treatment with hydrogen gas, wherein the hydrogen gas is at a pressure in the range of about 5 to about 50 psig, in the presence of a hydrogenation catalyst such as palladium on carbon, palladium hydroxide, platinum on carbon, tris(triphenylphosphine)rhodium(I) chloride (Wilkinson's catalyst), and the like, in the presence of an alcohol such as methanol, ethanol, and the like, to yield the corresponding compound of formula (Ih).

Compounds of formula (I) wherein L² is cis-C₂-C₈alkenyl may be prepared according to the process outlined in Scheme 10.

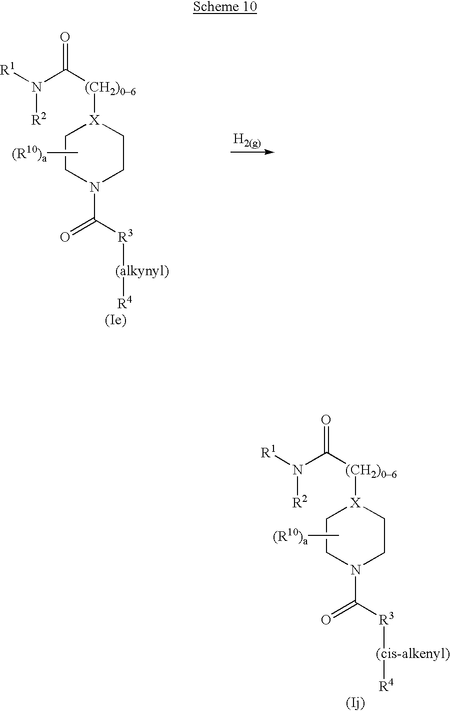

More particularly, a compound of formula (Ie), wherein L² is C₂-C₈alkynyl, prepared as in Scheme 5, is selectively reduced under hydrogenation conditions (i.e. by treatment with hydrogen gas, wherein the hydrogen gas is at a pressure in the range of about 2 to about 50 psig), in the presence of Lindlar's catalyst, in an organic solvent such as ethyl acetate, ethanol, and the like, to yield the corresponding cis-alkenyl compound of formula (Ij) Compounds of formula (I) wherein X is N, m is 1, L¹ is CH₂, Y¹ is C(O), and Y² is C(O) may alternatively be prepared according to the process outlined in Scheme 11.

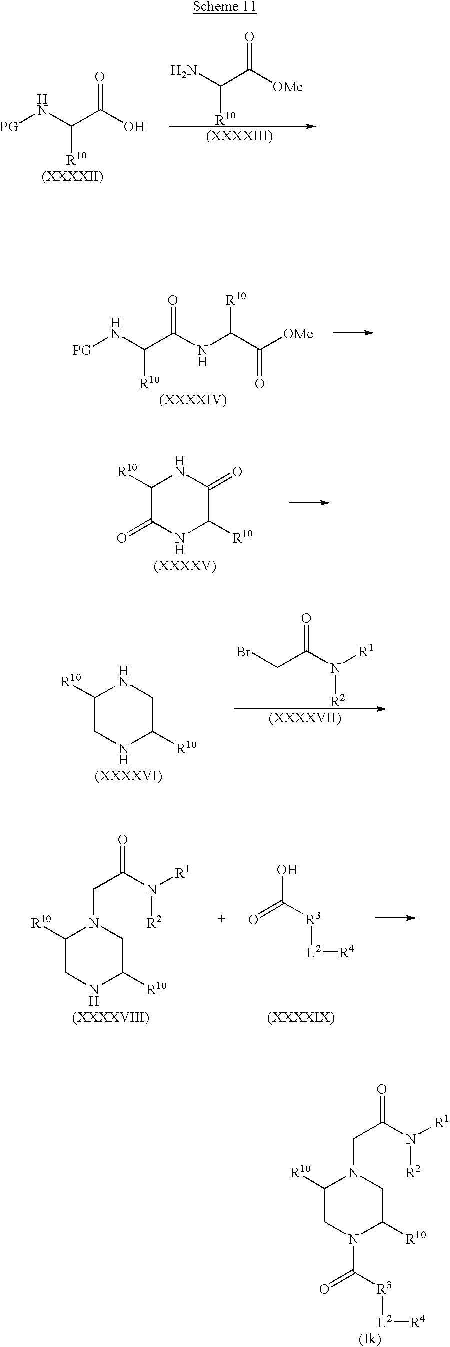

More particularly, an amino acid compound of formula (XXXXII), wherein PG is an amine protecting group such as tert-butoxycarbonyl, benzyloxycarbonyl, and the like, is reacted with a coupling agent, such as isobutylchloroformate, HATU, benzotriazol-1-yl-oxytris(dimethylamino)phosphonium hexafluorophosphate, and the like, in an organic solvent such as dichloromethane, chloroform, tetrahydrofuran, and the like, and then treated with a suitably substituted amino acid, a compound of formula (XXXXIII), such as glycine methyl ester, alanine methyl ester, phenylalanine methyl ester, and the like, wherein the R¹⁰ group on the compound of formula (XXXXII) and the R¹⁰ group on the compound of formula (XXXXIII) are each independently selected, to yield the corresponding compound of formula (XXXXIV).

The protecting group on the compound of formula (XXXXIV) is removed by known methods, for example, where PG is BOC, by treatment with an acid such as formic acid, acetic acid, trifluoroacetic acid, and the like and heating to an elevated temperature, preferably at a temperature in the range of about 95-110° C., in an organic solvent, such as a mixture of butanol, toluene, and the like to yield the corresponding compound of formula (XXXXV).

The compound of formula (XXXXV) is treated with a reducing agent, such as borane, lithium aluminum hydride, sodium borohydride, and the like, in an organic solvent, such as THF, diethyl ether, and the like, to yield the corresponding compound of formula (XXXXVI).

The compound of formula (XXXXVI) is reacted with a suitably substituted compound of formula (XXXXVII), in the presence of a base such as potassium tert-butoxide, sodium hydride, and the like, in an organic solvent such as THF, diethyl ether, and the like, to yield the corresponding compound of formula (XXXXVIII).

The compound of formula (XXXXVIII) is reacted with the compound of formula (XXXXIX), in the presence of a coupling agent such as oxalyl chloride, benzotriazol-1-yl-oxytris(dimethylamino)phosphonium hexafluorophosphate, HATU, and the like, in the presence of an organic base such as TEA, DIPEA, and the like, in an organic solvent such as methylene chloride, chloroform, THF, and the like, to yield the corresponding compound of formula (1k).

The compound of formula (XXXXIX) may be prepared according to the process outlined in Scheme 12.

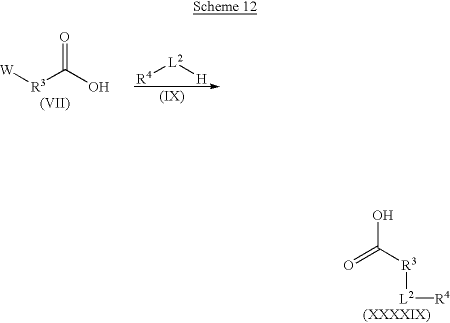

Specifically, a compound of formula (VII), wherein W is iodine, bromine, triflate, and the like, is reacted with a compound of formula (IX), wherein L² is a proximal alkene or proximal alkenyl, such as

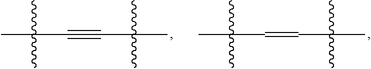

and the like, in the presence of a copper salt such as copper(I)iodide, copper(I)chloride, and the like, in the presence of a palladium catalyst such as palladium (II) chloride, palladium acetate, Pd(PPH₃)₄, and the like, in the presence of an organic base such as TEA, DEA, DIPEA, and the like, in an organic solvent such as DMF, DME, and the like, at an elevated temperature, preferably at a temperature in the range of about 80-130° C. to yield the corresponding compound of formula (XXXXIX).

Compounds of formula (I) wherein X is CH, m is 1, L¹ is CH₂, Y¹ is C(O), R¹ is H, Y² is C(O) and n is 0 (L² is absent), may alternatively be prepared according to the process outlined in Scheme 13.

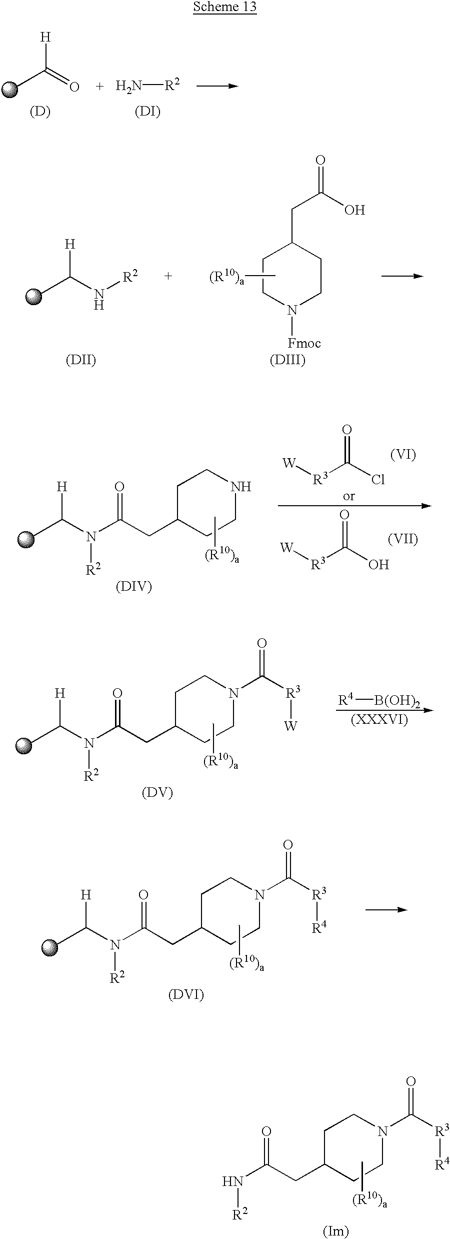

More specifically, an aldehyde terminate resin, a compound of formula (D), a known compound (for example FMPB Resin from Irori (substitution (1.02 mM/g))) is reacted with a primary amine, a compound of formula (DI), in an organic solvent such as DMF, DCE, DCM, and the like, in the presence of an acid such as HCl, TFA, acetic acid, and the like, and in the presence of a condensenation agent such as trimethyl orthoformate, molecular sieves, and the like, to yield the corresponding compound of formula (DII).

The compound of formula (DII) is reacted with Fmoc-(4-carboxymethyl)-piperidine, a compound of formula (DIII), a known compound or compound prepared by known methods, in the presence of a coupling agent such as 2-chloro-1,3-dimethylimidazolium chloride, HATU, and the like, optionally in the presence of a coupling additive, such as HOBT, HOAT, and the like, in the presence of an organic base such as TEA, DIPEA, and the like, in a solvent such as DMF, methylene chloride, DCE, and the like, and then de-protected with 25% piperidine in DMF, tetrabutylammonium fluoride in DMF, and the like, to yield the corresponding compound of formula (DIV).

The compound of formula (DIV) is reacted with a suitably substituted acid chloride, a compound of formula (VI), wherein W is iodine or bromine, in the presence of an organic base such as TEA, DIPEA, pyridine, and the like, in a halogenated solvent such as methylene chloride, DCE, and the like, to yield the corresponding compound of formula (DV).

Alternatively, the compound of formula (DIV) is reacted with a suitably substituted carboxylic acid, a compound of formula (VII), wherein W is iodine or bromine, in the presence of a coupling agent such as HATU, 2-chloro-1,3-dimethylimidazolium chloride, and the like, optionally in the presence of a coupling additive, such as HOBT, HOAT, and the like, in the presence of an organic base such as TEA, DIPEA, pyridine, and the like, in a solvent such as DMF, methylene chloride, DCE, and the like, to yield the corresponding compound of formula (DV).

The compound of formula (DV) is reacted with a suitably substituted boronic acid, a compound of formula (XXXVI), in the presence of a palladium catalyst such as palladium(II) acetate, tetrakis(triphenylphosphine) palladium(0), and the like, in the presence of a base such as TEA, potassium carbonate, sodium carbonate, and the like, in a solvent such as DMF, at an elevated temperature, preferably at temperature of about 80° C. to about 110° C., to yield the corresponding compound of formula (DVI).

The compound of formula (DVI) is cleaved from the solid support with a cleavage agent such as 25% trifluoroacetic acid in methylene chloride, DCE, and the like, at ambient temperatures to yield the corresponding compound of formula (Im).

Compounds of formula (I) wherein X is CH, m is 1, L¹ is CH₂, Y¹ is C(O), R¹ is H, Y² is C(O) and L² is C₂-C₈alkenyl or C₂-C₈alkynyl, may be prepared according to the process outlined in Scheme 14.

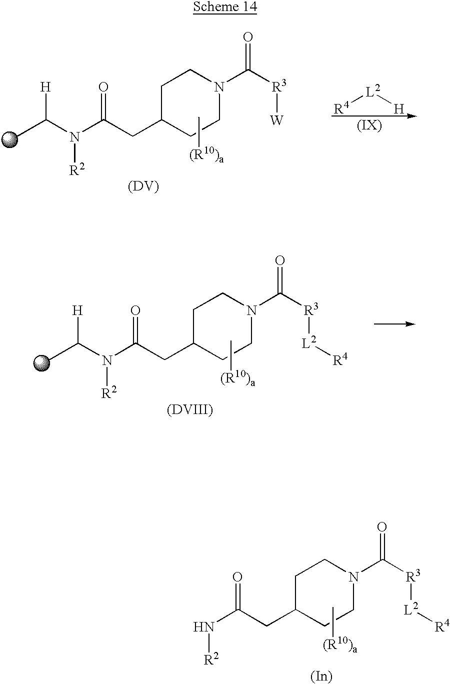

Accordingly, the compound of formula (DV), prepared as in Scheme 13, is reacted with a compound of formula (IX), wherein L² is a proximal alkenyl or proximal alkynyl, such as

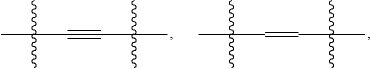

and the like, in the presence of a copper salt such as copper(I)iodide, and the like, in the presence of a palladium catalyst such as palladium (II) acetate, tetrakis(triphenylphosphine) palladium (0), and the like, in the presence of an organic base such as TEA, DEA, and the like, in an organic solvent such as DMF, toluene, dioxane, and the like, at an elevated temperature, preferably at a temperature of about 80° C. to about 110° C., to yield the corresponding compound of formula (DVIII).

The compound of formula (DVIII) is cleaved from the solid support with a cleaving cocktail such as 25% trifluoroacetic acid in methylene chloride, dichloroethane, and the like, at ambient temperatures to yield the corresponding compound of formula (In).

Compounds of formula (I) wherein X is CH, m is 1, L¹ is CH₂, Y¹ is C(O), R¹ is H, n is 1, L²is CH₂—NR⁵ and Y² is C(O) can be prepared according to the process outlined in Scheme 15.

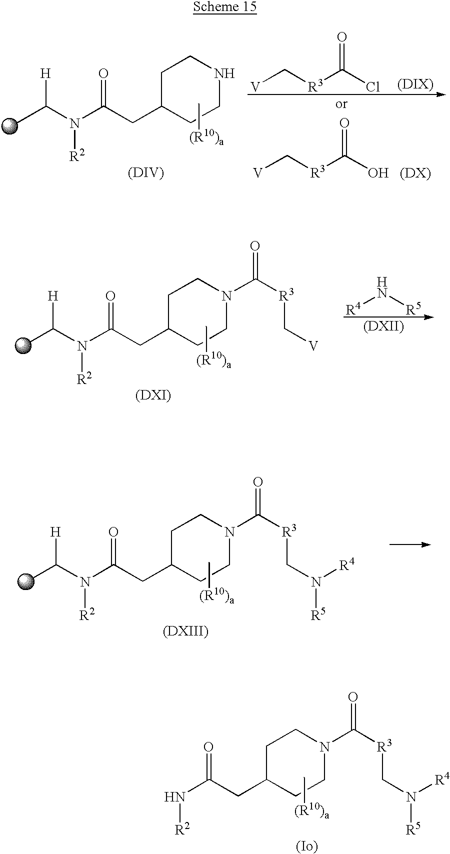

More specifically, a compound of formula (DIV), prepared as in Scheme 13, is reacted with a suitably substituted acid chloride, a compound of formula (DIX), wherein V is a leaving group such as bromide, chloride, O-tosyl, and the like, in the presence of an organic base such as TEA, DIPEA, cesium carbonate, and the like, in a halogenated solvent such as methylene chloride, DMF, DCE, and the like, to yield the corresponding compound of formula (DXI).

Alternatively, a compound of formula (DIV) is reacted with a suitably substituted carboxylic acid, a compound of formula (DX), wherein V is a leaving group such as bromide, chloride, O-tosyl, and the like, in the presence of a coupling agent such as HATU, 2-chloro-1,3-dimethylimidazolium chloride, and the like, optionally in the presence of a coupling additive, such as HOBT, HOAT, and the like, in the presence of an organic base such as TEA, DIPEA, pyridine, and the like, in a solvent such as DMF, methylene chloride, DCE, and the like, to yield the corresponding compound of formula (DXI).

The compound of formula (DXI) is reacted with an amine of formula (DXII), wherein R⁵ is as previously defined, in the presence of a base such as cesium carbonate, in a solvent such as DMF, DCM, DCE, and the like, to yield the corresponding compound of formula (DXIII).

The compound of formula (DXIII) is cleaved from the solid support with a cleaving cocktail such as 25% trifluoroacetic acid in methylene chloride, DCE, and the like, to yield the corresponding compound of formula (Io).

Compounds of formula (I) wherein X is CH, m is 1, L¹ is CH₂, Y¹ is C(O), R¹ is H, n is 1, L² is CH₂—O or CH₂—S and Y² is C(O) can be prepared according to the process outlined in Scheme 16.

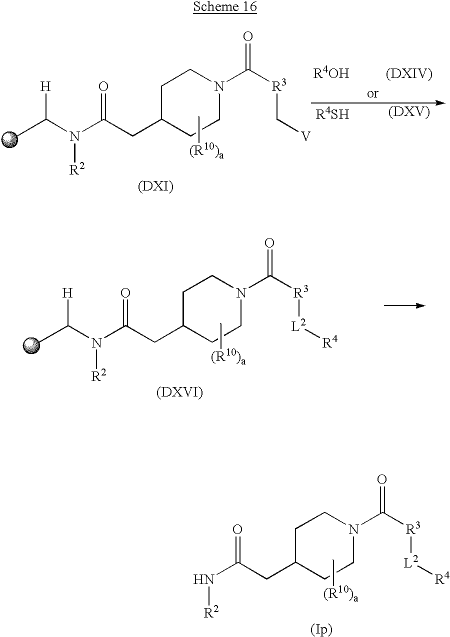

Accordingly, the compound of formula (DXI), prepared as in Scheme 15, is reacted with a compound of formula (DXIV) or a compound of formula (DXV), wherein R⁴ is as previously defined, in the presence of base such as sodium hydride, cesium carbonate, potassium t-butoxide, and the like, in a solvent such as DMF, DCM, N-methyl-morpholine, and the like, to yield the corresponding compound of formula (DXVI).

The compound of formula (DXVI) is cleaved from the solid support with a cleaving cocktail such as 25% trifluoroacetic acid in methylene chloride, dichloroethane, and the like, to yield the corresponding compound of formula (Ip).

When in the compound of formula (DXIII), prepared as in Scheme 15R⁵ is H, the amine portion of the compound of formula (DXIII) may be further optionally substituted to form a compound of formula (I) wherein L² is CH₂—NR⁵, wherein R⁵ is selected from C(O)-C₁₋₆alkyl, C(O)-aryl C(O)-aralkyl, C(O)-heteroaryl or C(O)-heterocycloalkyl, according to the process outlined in Scheme 17.

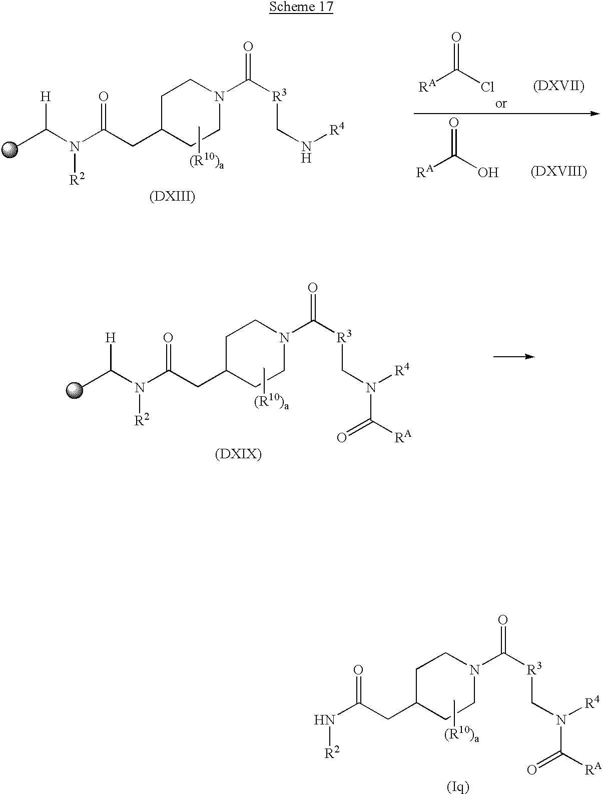

More specifically, the compound of formula (DXIII), prepared as in Scheme 15, is reacted with a suitably substituted acid chloride, a compound of formula (DXVII), wherein RA is selected from the group consisting of C₁₋₆alkyl, aryl, aralkyl, heteroaryl and heterocycloalkyl, wherein the aryl, aralkyl, cycloalkyl, heteroaryl or heterocycloalkyl may be optionally substituted with one or more substituents independently selected from halogen, hydroxy, C₁-C₆alkyl, C₁-C₆ alkoxy, halogenated C₁-C₆alkyl, halogenated C₁-C₆alkoxy, nitro, cyano, amino, C₁-C₄alkylamino or di(C₁-C₄alkyl)amino, in the presence of base such as TEA, DIPEA, pyridine, and the like, in a halogenated solvent such as methylene chloride, dichloroethane, and the like, to yield the corresponding compound of formula (DXIX).

Alternatively, the compound of formula (DXIII) is reacted with a suitably substituted carboxylic acid, a compound of formula (DXVIII), wherein RA is as previously defined, in the presence of a coupling agent such as DIC, 2-chloro-1,3-dimethylimidazolium chloride, HOAT, and the like, optionally in the presence of coupling additives, such as HOBT, HOAT, and the like, in the presence of an organic base such as TEA, DIPEA, pyridine, and the like, in a solvent such as DMF, methylene chloride, dichloroethane, and the like, to yield the corresponding compound of formula (DXIX).

The compound of formula (DXIX) is cleaved from the solid support with a cleaving cocktail such as 25% trifluoroacetic acid in methylene chloride, dichloroethane, and the like, to yield the corresponding compound of formula (Iq).

When in the compound of formula (DXIII), prepared as in Scheme 15, R⁵ is H, the amine portion of the compound of formula (DXIII) may alternatively be further optionally substituted according to the process outlined in Scheme 18.

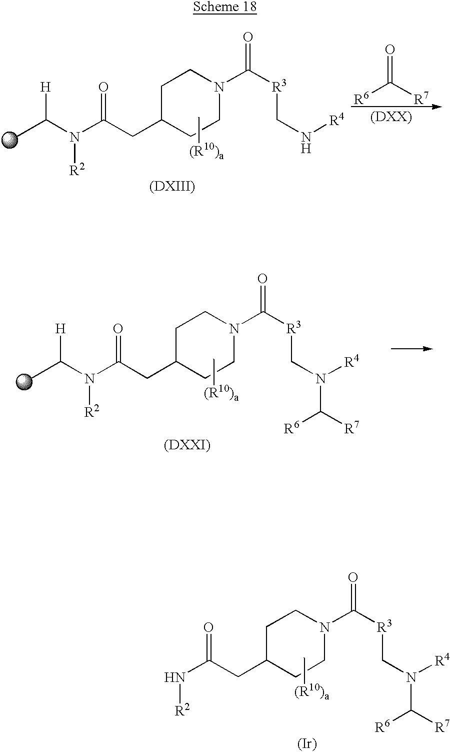

Accordingly, the compound of formula (DXIII), prepared as in Scheme 15, is reacted with a compound of formula (DXX), wherein R⁶ and R⁷ are as previously defined, in a solvent such as DMF, DCM, DCE, and the like, in the presence of an acid such as acetic acid, TFA, and the like, in the presence of an additive such as TMOF, molecular sieves, and the like, in the presence of a reducing agent such as sodium triacetoxyborohydride, sodium cyanoborohydride, and the like, to yield the corresponding compound of formula (DXXI).

The compound of formula (DXXI) is cleaved from the solid support with a cleaving cocktail such as 25% trifluoroacetic acid in methylene chloride, dichloroethane, and the like, to yield the corresponding compound of formula (Ir).

Compounds of Formula (I) wherein X is CH, m is 1, L¹ is CH₂, Y¹ is C(O), Y² is C(O), R³ is phenyl, n is 1 and L² is NH—CH₂, may be prepared according to the process outlined in Scheme 19.

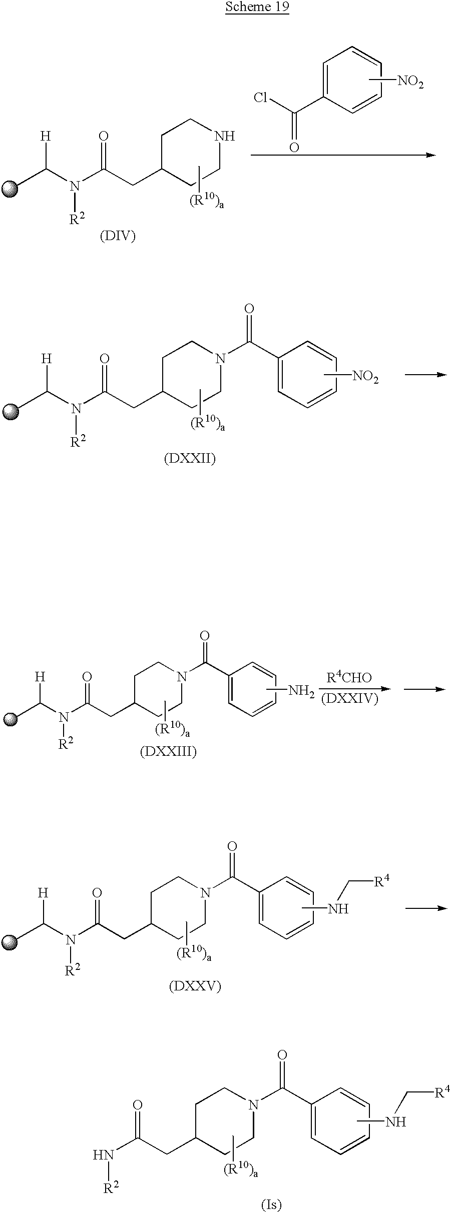

More particularly, a compound of formula (DIV), prepared as in Scheme 13 is reacted with nitrobenzoyl chloride, wherein the nitro group is bound at the 2, 3, or 4 position, in an amount in the range of about 3 to about 8 equivalents, preferably about 5 equivalents, in the presence of an organic base such as pyridine, TEA, DIPEA, and the like, wherein the base is present in an amount in the range of about 3 to about 8 equivalents, preferably about 6 equivalents, in a halogenated solvent such as methylene chloride, chloroform, and the like, to yield the corresponding compound of formula (DXXII).

The compound of formula (DXXII) is reduced by treatment with a reducing agent such as tin(II)chloride, NaBH₄, ferric chloride, and the like, in an organic solvent such as DMF, N-methylpyrrolidinone, in the presence of about 1% by volume water, to yield the corresponding compound of formula (DXXIII).

The compound of formula (DXXIII) is reacted with a suitably substituted aldehyde of formula (DXXIV), wherein the aldehyde is present in an amount in the range of about 5 to about 15 equivalents, preferably about 10 equivalents, in a solvent mixture such as DCE/TMOF, DCMITMOF, DMF/TMOF, and the like; then washed with an organic solvent such as DCE, DMF, and the like, preferably DCE (to remove excess compound of formula (DXXIV)); and then treated with a reducing agent such as NaBH(OAc)₃, in an amount in the range of about 3 to about 8 equivalents, preferably about 5 equivalents, in an organic solvent such as DCE, chloroform, and the like, to yield the corresponding compound of formula (DXXV).

The compound of formula (DXXV) is cleaved from the solid support with a cleaving cocktail such as 50% TFA in DCM, and the like, to yield the corresponding compound of formula (Is).

Optionally, the compound of formula (Is) is further reacted with an acid chloride, a compound of the formula R⁵—C(O)Cl, a compound of formula (DVII), such as acetyl chloride, benzoyl chloride, and the like, in the presence of an organic base such as TEA, DIPEA, pyridine, and the like, in a halogenated solvent such as methylene chloride, dichloroethane, and the like, to further substituted the terminal secondary amine group.

Compounds of formula (I) wherein m is 1, L¹ is CH₂, Y¹ is C(O), R¹ is hydrogen, Y² is C(O), n is 1 and L² is C(O) may be prepared according to the process outlined in Scheme 20.

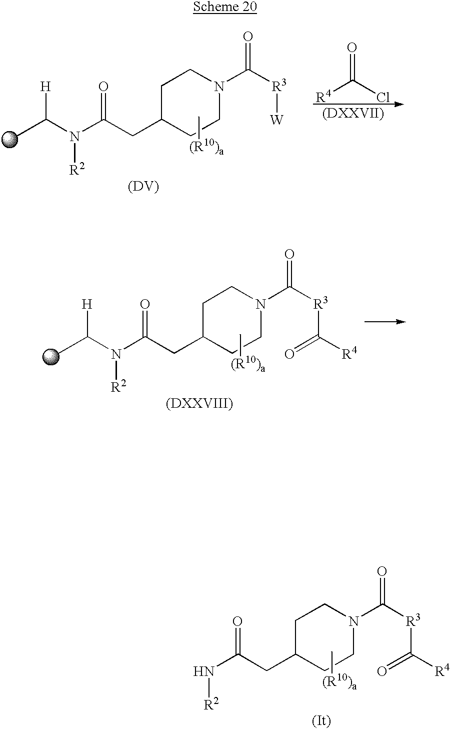

More particularly, a compound of formula (DV), prepared as in Scheme 13, is reacted with fine mesh magnesium metal, preferably in the presence of an additive such as zinc chloride, tetrakis(triphenylphosphine) palladium(0), and the like, preferably zinc chloride, in a solvent such as diethyl ether, THF, and the like, at a temperature sufficient to initiate organomagnesium halide formation, and then reacted with a suitably substituted acid chloride, a compound of formula (DXXVII), to yield the corresponding compound of formula (DXXVIII).

The compound of formula (DXXVIII) is cleaved from the solid support with a cleavage agent such as 25% trifluoroacetic acid in methylene chloride, DCE, and the like, at about ambient temperature, to yield the corresponding compound of formula (It).

Compounds of formula (I) wherein Y¹ is C(O), m is 1, L¹ is CH₂, Y² is C(O), R³ is phenyl, n is 1 and L² is NH—CH₂ may be prepared according to the process outlined in Scheme 21.

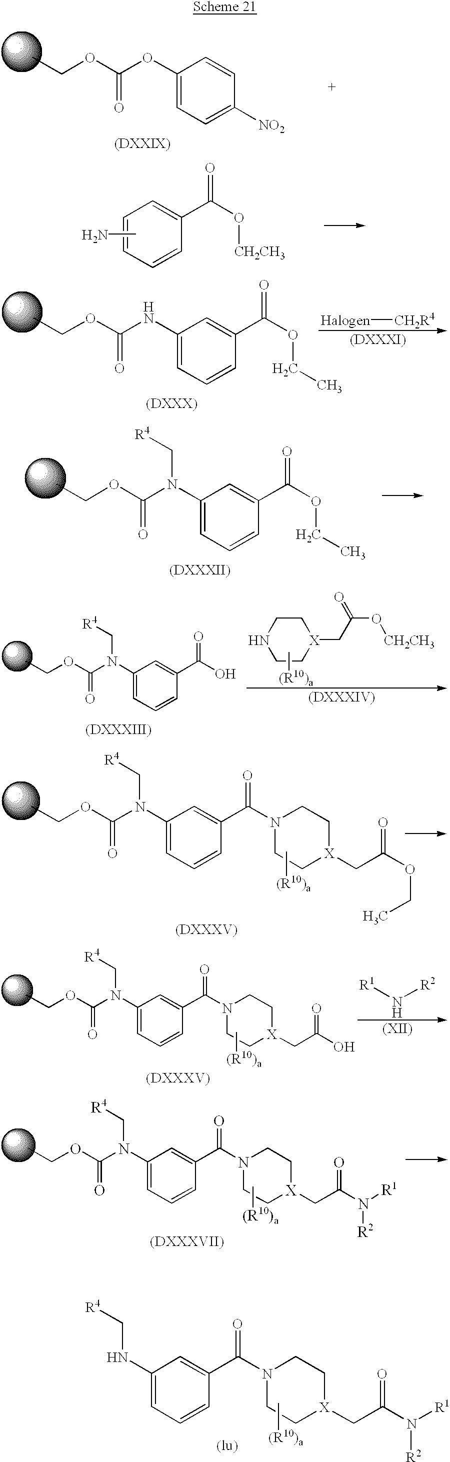

More particularly, a commercially available resin of formula (DXXIX) is reacted with a suitably substituted aminobenzoic ester, (wherein the amino group is bound at the 2, 3, or 4 position), wherein the aminobenzoic ester is present in an amount in the range of about 5 to about 15 equivalents, preferably about 10 equivalents, in the presence of an additive such as HOBT, N, O-bis(trimethylsilyl)acetamide with DMAP, and the like, wherein the catalyst is present in an amount in the range of about 3 to about 8 equivalents, preferably about 5 equivalents, and in the presence of an organic base such as DIPEA, TEA, pyridine, and the like, wherein the organic base is present in an amount in the range of about 5 to about 15 equivalents, preferably about 10 equivalents, in a solvent mixture such a DCM/NMP, DCM/THF, and the like, preferably DCM/NMP at 67%133% (v/v), to yield the corresponding compound of formula (DXXX).

The compound of formula (DXXX) is reacted with a strong base such as NaH, t-butylONa, and the like, preferably NaH, wherein the base is present in an amount in the range of about 2 to about 4 equivalents, preferably about 3 equivalents, in an organic solvent such as DMF, NMP, and the like, and then reacted with about 5 to about 15 equivalents of a compound of formula (DXXXI), wherein R⁴ is as previously defined, preferably about 10 equivalents, to yield the corresponding compound of formula (DXXXII).

The compound of formula (DXXXII) is hydrolyzed with an aqueous base such as NaOH, sodium carbonate, and the like, preferably NaOH, in the presence of an organic solvent such as DME, THF, and the like, preferably DME, at a temperature in the range of about 25-80° C., preferably at about 55° C., to yield the corresponding compound of formula (DXXXIII).

The compound of formula (DXXXIII) is coupled with a suitably substituted compound of formula (DXXXIV), in the presence of a coupling agent such as DIC, HATU/DIPEA, and the like, preferably HATU/DIPEA, in an organic solvent such as DMF, NMP, and the like, preferably NMP, to yield the corresponding compound of formula (DXXXV).

The compound of formula (DXXXV) is hydrolyzed with an aqueous base such as NaOH, sodium carbonate, and the like, preferably NaOH, in the presence of an organic solvent such as DME, THF, and the like, preferably DME, at a temperature in the range of about 25-80° C., preferably at about 55° C., to yield the corresponding compound of formula (DXXXVI).

The compound of formula (DXXXVI) is reacted with a suitably substituted compound of formula (XII), wherein R¹ and R² are as previously defined, in the presence of a coupling agent such as DIC, HATU/DIPEA, and the like, preferably HATU/DIPEA, in an organic solvent such as DMF, NMP, and the like, preferably NMP, to yield the corresponding compound of formula (DXXXVII).

The compound of formula (DXXXVII) is cleaved from the solid support with an acidic cleaving cocktail such as 50% trifluoroacetic acid in methylene chloride, to yield the corresponding compound of formula (Iu).

Compounds of formula (I) wherein Y¹ and Y² are each C(S) may be prepared by reacting the corresponding compound of formula (I) wherein Y¹ and Y² are each C(O) with Lawesson's reagent (2,4-bis(4-methoxyphenyl)-1,3-dithia-2,4-diphosphetane-2,4-disulfide), in the presence of a solvent such as toluene, xylene, and the like.

Compounds of formula (I) wherein one of Y¹ or Y² is C(S) may be prepared by reacting a suitably substituted intermediate, wherein one of Y¹ or Y² is C(O) with Lawesson's reagent, in the presence of a solvent such as toluene, xylene, and the like, to yield the corresponding intermediate wherein said Y¹ or Y² is C(S) and then further reacting the intermediate compound according to the processes previously disclosed to yield the desired compound of formula (I).

One skilled in the art will recognize that compounds of formula (I) wherein R³ is selected from substituted aryl, substituted aralkyl, substituted heteroaryl or substituted heterocycloalkyl and the substituent on the aryl, aralkyl, heteroaryl or heterocycloalkyl group is —(L²)ₙ—R⁴ may be prepared by coupling a dibromo- or diiodobenzoyl chloride or a dibromo- or diiodo-benzoic acid to a suitably substituted piperazine or piperidine in the manner as previously described and then reacting the dibromo- or diiodo-product with at least 2 molar equivalents of either a compound of formula (XXXVI) (i.e. an R⁴-boronic acid), as described in Scheme 7 or a compound of formula (IX) (i.e. a compound of the formula R⁴—L²—H) as described in Scheme 1.

One skilled in the art will recognize that a multitude of diverse compounds of the present invention may be prepared by coupling onto the

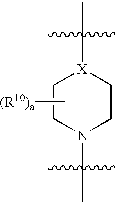

moiety the —(L¹)ₘ—Y¹—NR¹R² and —Y²—R³—(L²)—R⁴ portions of the compound, by selectively combining the steps for coupling the desired —(L¹)ₘ—Y¹—NR¹R² portion with steps for coupling the desired —Y²—R³—(L²)ₙ—R⁴ portions.

The present invention therefore provides a method of treating nervous system disorders in a subject in need thereof which comprises administering any of the compounds as defined herein in a quantity effective to treat said disorder. The compound may be administered to a patient by any conventional route of administration, including, but not limited to, intravenous, oral, subcutaneous, intramuscular, intradermal and parenteral. The quantity of the compound which is effective for treating a nervous system disorder disorder is between 0.1 mg per kg and 200 mg per kg of subject body weight.

The present invention also provides pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 5 to about 1000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellu lose, methylcellu lose, polyvinyl-pyrrolidone or gelatin.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The method of treating a nervous system disorder described in the present invention may also be carried out using a pharmaceutical composition comprising any of the compounds as defined herein and a pharmaceutically acceptable carrier. The pharmaceutical composition may contain between about 5 mg and 1000 mg, preferably about 10 to 500 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms may include suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phophatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyl-eneoxidepolylysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Compounds of this invention may be administered in any of the foregoing compositions and according to dosage regimens established in the art whenever treatment of a nervous system disorder is required.

The daily dosage of the products may be varied over a wide range from 5 to 1,000 mg per adult human per day. For oral administration, the compositions are preferably provided in the form of tablets containing, 5.0, 10.0, 15.0, 25.0, 50.0, 100, 250 and 500 milligrams of the active ingredient for the symptomatic adjustment of the dosage to the patient to be treated. An effective amount of the drug is ordinarily supplied at a dosage level of from about 0.1 mg/kg to about 200 mg/kg of body weight per day. Preferably, the range is from about 0.2 mg/kg to about 100 mg/kg of body weight per day, and especially from about 0.5 mg/kg to about 75 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

The following Examples are set forth to aid in the understanding of the invention, and are not intended and should not be construed to limit in any way the invention set forth in the claims which follow thereafter.

Unless otherwise indicated, ¹H NMRs were run on either a Bruker Avance 300 MHz NMR spectrometer or on a Bruker AC-300 MHz NMR spectrometer. Calculated molecular weight numbers represent an average based on isotopic abundance and measured molecular weights were determined on a Micromass Platform LC LC/MS mass spectrometer equipped with an electrospray ion source.

### EXAMPLE 1

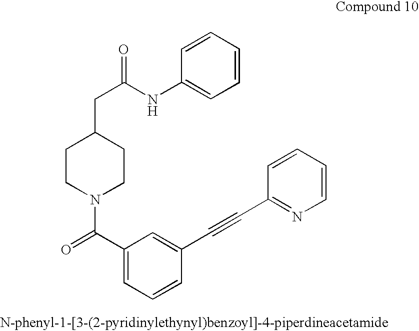

Step A:

To a solution of 1-benzylpiperidone (25 g, 0.132 mol) in toluene (300 mL) under nitrogen at RT was added (carbethoxymethylene)triphenylphosphorane (48 g, 0.138 mol). The reaction mixture was heated to reflux and allowed to stir at reflux overnight. The reaction mixture was allowed to cool to RT and the toluene was removed by rotary evaporation. The resulting crude oil was purified by column chromatography using a gradient of 0 to 20% EtOAc/Hexanes as the elution solvent to yield the product as a yellow oil.

Step B:

To a solution of the product prepared in Step A, (21 g, 0.081 mol) in EtOH (100 mL), in a hydrogenation bottle that had been flushed with nitrogen, was added Pearlman's catalyst (palladium hydroxide, 20 wt. % Pd (dry basis) based on carbon) (2.1 g, 10 wt. %). The solution was subjected to hydrogen in a Parr shaker at 50 psig for 15 h. The suspension was filtered through Celite and the EtOH removed by rotary evaporation to yield the product as a colorless liquid.

Step C:

To a solution of the product prepared in Step B (16.3 g, 0.095 mol) in methylene chloride (300 mL) under nitrogen at 0° C. was added triethylamine (27 mL, 0.2 mol) and 3-bromobenzoyl chloride (13.9 mL), 0.1 mol). The solution was allowed to warm to RT and stirred for 2 h. The methylene chloride was removed in vacuo and the residue was partitioned between water (300 mL) and EtOAc (500 mL). The layers were separated and the organic layer was washed with brine (500 mL), dried over Na₂SO₄, filtered and concentrated via rotary evaporation. The crude oil was then purified by column chromatography eluting with a gradient of 0 to 20% EtOAc/Hexanes to yield the product as an orange oil.

Step D:

A mixture of compound prepared in Step C (20 g, 0.056 mol), 2-ethynylpyridine (7.6 g, 0.073 mol), Cul (2 g), bis-triphenylphosphinepalladium (II) chloride (2 g, 5 mol %), triethylamine (12 mL) and DMF (50 mL) was heated at 130° C. in a sealed pressure tube for 48 h. The reaction mixture was allowed to cool to RT and was then partitioned between water (200 mL) and EtOAc (200 mL). The particulate solution was filtered through Celite and the layers were separated. The aqueous solution was extracted with EtOAc (2×200 mL). The combined organic layers were washed with brine (4×100 mL), dried over Na₂SO₄, filtered and concentrated via rotary evaporation. The residue was purified by column chromatography eluting with 1:1 EtOAc/Hexanes to yield the product as a dark oil.

Step E:

To a solution of compound prepared in Step D (8 g, 0.02 mol) in THF (200 mL) at RT was added a solution of LiOH (1.01 g, 0.04 mol) in water (100 mL). The reaction mixture was allowed to stir at RT overnight. The solution was acidified by the addition of citric acid (8 g, 0.04 mol) and extracted with EtOAc (2×200 mL). The organic layer was dried over Na₂SO₄, filtered and concentrated via rotary evaporation to yield the product as a dark oil.

Step F:

To a solution of compound prepared in Step E (6 g, 0.017 mol) in methylene chloride (150 mL) at RT under nitrogen was added aniline (1.7 mL, 0.018 mL) and triethylamine (4.8 mL, 0.035 mol). The solution was cooled to 0° C. and then isobutyl chloroformate (2.6 mL, 0.02 mol) was added. The reaction mixture was allowed to warm to RT and was stirred for 30 min. The methylene chloride was removed in vacuo and to the residue was added EtOAc (300 mL). The organic solution was washed with brine (300 mL), dried over Na₂SO₄, filtered and concentrated via rotary evaporation. The residue was purified by column chromatography eluting with 1:1 EtOAc/Hexanes to yield the title product as a brown oil.

Step G:

To the crude product prepared in Step F was added EtOAc (100 mL) and 1N HCl in diethyl ether (15 mL, 0.15 mol). The volatiles were removed in vacuo and the resultant solid dried over vacuum to yield the title compound as a HCl salt.

¹H NMR (300 MHz, CD₃OD): δ 1.23-1.34 (m, 2H), 1.79 (d, J=0.03 Hz, 1H), 1.95 (d, J=0.81 MHz, 1H), 2.17-2.22 (m, 1H), 2.38 (t, J=0.64,1.83 Hz, 2H), 2.95 (m, 1H), 3.21 (m, 1H), 3.69 (m, 1H), 4.65 (m, 1H), 7.10 (t, 1H, J=2.24, 3.39 Hz, 1H), 7.31 (t, J=3.19, 3.75 Hz, J=3.19, 2H), 7.55 (d, J=1.29 Hz, 2H), 7.62 (d, J=0.16 Hz, 2H), 7.79 (s, 1H), 7.82-7.86 (m, 1H), 8.05 (m, 1H), 8.26 (d, J=0.90 Hz, 1H), 8.64 (t, J=2.58, 2.70 Hz, 2H), 8.87 (d, J=0.1 Hz, 1H).

MH⁺ 424.25.

### EXAMPLE 2

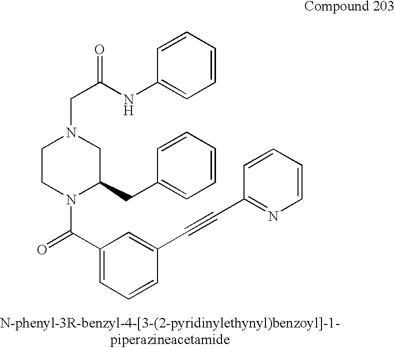

Step A:

N-(tert-Butoxycarbonyl)-D-phenylalanine (2.00 g, 7.54 mmol) was dissolved in dry dichloromethane (50 mL). Triethylamine (1.91 g, 18.85 mmol) and then isobutylchloroformate (1.03 g, 7.54 mmol) were added and the solution was stirred at room temperature for 10 minutes. Glycine methyl ester hydrochloride (1.14 g, 9.05 mmol) was added and the mixture was stirred overnight. The reaction was poured into a separatory funnel and washed successively with aqueous hydrochloric acid (1.0 N, 50 mL), saturated aqueous sodium bicarbonate, and brine. The organic phase was concentrated under vacuum to a colorless oil which was dissolved in formic acid (100 mL). After stirring for two hours at room temperature, the solution was evaporated under vacuum to provide a yellow oil which was dissolved in a solution of 2-butanol (50 mL) and toluene (50 mL). The mixture was boiled in an unstoppered flask, with the solvent level maintained by the occasional addition of 2-butanol. The reaction was then cooled and stored at −20° C. overnight. The resulting white precipitate was collected by vacuum filtration to yield the diketopiperazine product.

Step B:

(As described by Jung et al. in J. Org. Chem., 1985, 50, 4909-4913)

The diketopiperazine compound prepared in Step A (0.640 g, 3.13 mmol) was added to a stirred solution of borane-THF (1.0 M in THF, 31.3 mL, 31.3 mmol). The reaction was stirred for 4 days at room temperature and then quenched by the slow addition of aqueous sodium hydroxide (1.0 N). The solution was extracted with dichloromethane, dried, concentrated under vacuum, and chromatographed (silica, 10:90 methanol:dichloromethane) to yield the (R)-2-benzylpiperazine product.

Step C:

The compound prepared in Step B (0.354 g, 2.01 mmol) was dissolved in dry THF (10 mL). Potassium tert-butoxide (1.0 M in THF, 2.21 mL, 2.21 mmol) was added and the solution was stirred at room temperature for one hour. 2-Bromo-N-phenylacetamide (0.516 g, 2.41 mmol) was added to the solution. After about 5 hours, the reaction was diluted with diethyl ether and water. The solution was extracted with diethyl ether. The combined organic solution was dried, concentrated, and chromatographed (silica, 95:5 dichlormethane:methanol) to yield the product as an off-white solid.

Step D:

3-lodobenzoic acid (1.48 g, 5.97 mmol) and 2-ethynylpyridine (0.923 g, 8.95 mmol) were added to a solution of triethylamine (4 mL) and DMF (4 mL). N₂ gas was bubbled through the solution for 10 minutes. Bis-triphenylphosphinepalladium (II) chloride and copper (I) iodide were added. The solution was heated to about 150° C. under reflux overnight. The reaction was cooled, concentrated under vacuum to about 1 mL, diluted with ethyl acetate (100 mL) and washed with brine. The organic solution was extracted with aqueous sodium hydroxide (1 N, 100 mL). The combined basic extracts were neutralized with concentrated sulfuric acid and then extracted with dichloromethane. The organic extracts were dried and concentrated to yield the product as a brown powder.

Step E:

To a solution of the compound prepared in Step D (0.015 g, 0.066 mmol) in dichloromethane (1 mL) was added triethylamine (0.008 g. 0.083 mmol) and then oxalyl chloride (2.0 M in dichloromethane, 0.033 mL, 0.066 mmol). The dark solution was stirred at room temperature for 2 hours and then the compound prepared in Step C (0.017 g, 0.055 mmol) was added. The reaction was stirred at room temperature overnight. The reaction was transferred directly to a preparative TLC plate for purification (5:95 methanol:dichloromethane). The purified product was dissolved in diethyl ether and hydrochloric acid (1 M solution in diethyl ether, 0.1 mL) was added. The mixture was then concentrated to dryness to yield the product as a white powder, as its hydrochloride salt.

¹H NMR (300 MHz, CD₃OD): δ 2.9-3.1 (m, 1H), 3.3-4.0 (m, 8H), 4.2-4.4 (m, 2H), 7.0-7.9 (m, 14H), 8.00 (d, J=5.9 Hz, 1H), 8.22 (m, 1H), 8.56 (m, 1H), 8.86 (br s, 1H).

MH⁺ 515.37.

### EXAMPLE 3

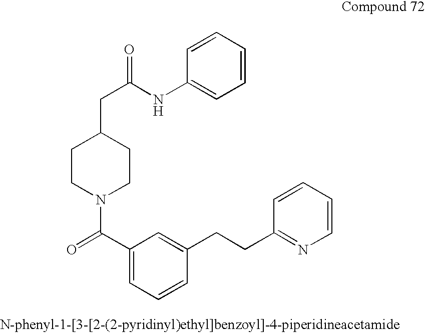

To a solution of the compound prepared as in Example 1 (0.5 gm, 1.2 mmol) in ethanol (20 ml), was added Pd/carbon (10%) (0.1 gm) under N₂. The resulting mixture was subjected to hydrogen at 20 psig in a Parr Shaker for 2 h. The mixture was vacuum filtered through Celite and the filtrate concentrated via rotary evaporation to yield the reduced product as an oil. The oil was treated with 1 N HCl/ether (1.2 ml) to yield the product as a crystalline HCl salt.

¹H NMR (300 MHz, CD₃OD): δ 1.29-1.69 (m, 2H), 1.73-1.86 (m, 2H), 2.1-2.3 (m, 1H), 2.36 (m, 2H), 2.88-2.91 (m, 1H), 3.10-3.21 (m, 2H), 3.30-3.43 (m, 3H), 3.60-3.64 (m, 1H), 4.59-4.63 (m, 1H), 7.07 (t, J=7.43 Hz, 1H), 7.26-7.41 (m, 6H), 7.55 (d, 2H, J=8.33 Hz, 2H), 7.88-7.96 (m, 2H), 8.51 (t, J=6.75 MHz, 1H), 8.74 (d, J=5.45 MHz, 1H).

MH⁺ 428.33

### EXAMPLE 4

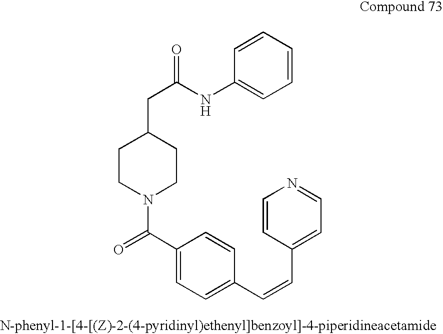

Step A:

To an ice cooled solution of piperidine ester (12 gm, 0.07 mol) in methylene chloride (100 ml) was added TEA (19 ml) and 4-iodo acetyl chloride (20 gm, 0.077 mol). The resultant mixture was stirred at room temperature for 30 min. The mixture was filtered and the filtrate concentrated via rotary evaporation. The residue was purified by column chromatography on silica eluting with 20/80 ethyl acetate/hexane to yield the product as an oil.

Step B:

Iodobenzoyl piperidine (6 gm, 0.015 mol) from Step A, 4-ethynyl pyridine (2.0 gm, 0.02 mol), Cul (0.3 gm, 5%wt.) and bis triphenyl phosphine plladium dichloride (0.54 gm, 5%mol) were placed into a sealed tube with TEA/DMF(5/5 ml). The resultant mixture was stirred at 110° C. for 3.5 hours. The mixture was partitioned between Ethyl acetate (300 ml) and water (100 ml). The Ethyl acetate layer was separated, washed with brine, dried over Na₂SO₄, filtered and concentrated via rotary evaporation. The residue was purified by column chromatography on silica eluting with ethyl acetate to yield the product as an orange oil.

Step C:

To a solution of piperidine ester (0.8 gm, 2.1 mmol) from Step B in ethanol (20 ml) was added Lindlar's catalyst (0.16 g). The resulting mixture was subjected to hydrogen at 3 psi for 24 hours in a Parr shaker. The mixture was vacuum filtered through Celite and the filtrate concentrated via rotary evaporation to yield a mixture of the desired cis-alkene product, the alkyne starting material and the fully reduced alkyl product. The mixture was carried without purification.

Step D:

To a solution of mixture from Step C (0.68 gm, 0.0018 mol) in THF/H₂O was added LiOH (0.086 gm, 0.0036 mol) and the resultant solution was allowed to stir at room temperature overnight. Citric acid (0.7 gm) added and the mixture was stirred for another 30 min. The solution was then extracted with ethyl acetate (100 ml). The ethyl acetate layer was separated, dried over MgSO₄, filtered and concentrated via rotary evaporation to yield the product as a yellow solid.

Step E:

To a solution of the product from Step D (0.1 gm, 0.28 mmol) in CH₂Cl₂/TEA (4 ml/0.08 ml) was added isobutyl chloroformate (0.04 ml, 0.31 mmol) followed by aniline (0.03 gm, 0.31 mmol). The mixture was stirred at room temperature for 15 min. The crude mixture was immediately placed on a prep TLC plate and purified yield the cis-alkene product.

¹H NMR (300 MHz, CDCl₃): δ 1.18-1.36 (m, 2H), 1.69-1.94 (m, 2H), 2.10-2.15 (m, 1H), 2.28-2.37 (m, 2H), 2.80-2.94(m, 1H), 3.06-3.17 (m, 1H), 3.62-3.71 (m, 1H), 4.53-4.61 (m, 1H), 6.90 (d, J=11.76 Hz, 1H), 7.08 (d, J=11.76 Hz, 1H), 7.28-7.61 (m, 9H), 67.81 (d, J=5.4 Hz, 2H), 8.62 (d, J=5.80 Hz, 2H).

MH⁺ 426.27.

### EXAMPLE 5

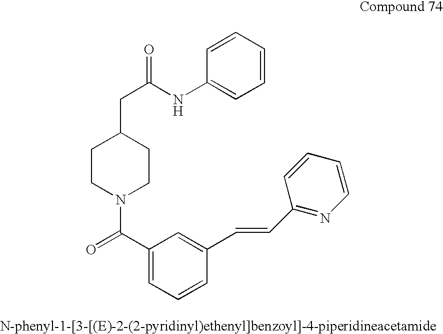

Step A:

To a solution of iodobenzoyl piperidine (3.0 g, 7.5 mmol) in DMF (50 ml) at room temperature was added TEA (50 ml), bis(acetato)bis(triphenylphosphine)Pd(II) (0.25 g, 4%mol) and 4-vinyl pyridine (1.57 ml, 15 mmol). The resulting solution was heated in a sealed tube at 100° C. for 48 hours. The solution was cooled to room temperature and poured into 100 ml water. The solution was extracted with ethyl acetate (200 ml). The ethyl acetate layer was separated, washed with brine (100 ml×2), dried over sodium sulfate, filtered and concentrated via rotary evaporation. The resulting crude oil was purified by column chromatography eluting with ethyl acetate to yield the product as an orange oil.

Step B:

To a solution of alkenyl piperidine (1.1 gm, 2.9 mmol) from Step A in THF (30 ml) and water(20 ml), was added LiOH (0.14 gm, 5.8 mmol) and the resultant solution was stirred at room temperature overnight. Citric acid (1.4 gm) was added and stirring was continued for 10 min. The solution was extraced by ethyl acetate (100 ml). The ethyl acetate layer was dried over sodium sulfate and concentrated to yield the product as a yellow solid.

Step C:

To a solution of the product prepared in Step B (0.1 gm, 0.28 mmol) in CH₂Cl₂/TEA(4 ml/0.08 ml) was added isobutyl chloroformate (0.04 ml, 0.31 mmol) followed by aniline (0.03 gm, 0.31 mmol). The mixture was stirred at room temperature for 15 min. The crude mixture was immediately purified by preparative TLC to yield the product, which was converted to its HCl salt upon treatment with 1 M HCl/Et₂O.

Yield: 0.07 g (58%).

¹H NMR (300 MHz, CD₃OD): δ 1.20-1.35(m, 2H), 1.71-1.93 (m, 2H), 2.11-2.18 (m, 1H), 2.28-2.37 (m, 2H), 2.86-2.98 (m, 1H), 3.10-3.21 (m, 1H), 3.65-3.77 (m, 1H), 4.60-4.69 (m, 1H), 7.07 (t, J=7.4 Hz, 1H), 7.39 (t, J=7.6 Hz, 2H), 7.44 (d, J=16.3 Hz, 1H), 7.50-7.58 (m, 5H), 7.76 (s, 1H), 7.80-7.90 (m, 2H), 7.99 (d, J=16.3 Hz, 1H).

MH⁺ 426.30.

### EXAMPLE 6

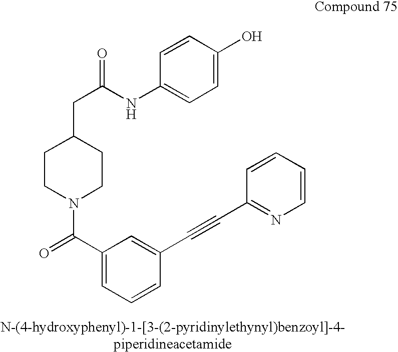

To a solution of N-phenyl-1-[3-(2-pyridinylethynyl)benzoyl]-4-piperdineacetamide (0.3 gm, 0.86 mmol), prepared as in Example 1, in CH₂Cl₂/TEA (4 ml/0.24 ml) was added isobutyl chloroformate (0.12 ml, 0.9 mmol) followed by 4-aminophenol (0.1 gm, 0.9 mmol). The mixture was stirred at room temperature for 15 min. The crude mixture was purified by preparative TLC to yiled the product, which was converted to an HCl salt upon treatment with 1M HCl/Et₂O.

¹H NMR (300 MHz, DMSO): δ 1.14-1.25(m, 2H), 1.60-1.79 (m, 2H), 2.00-2.08 (m, 1H), 2.19-2.23 (m, 2H), 2.77-2.86 (m, 1H), 3.01-3.11 (m, 1H), 3.49-3.80 (m, 1H), 4.38-4.50 (m, 1H), 6.66 (d, J=8.82 Hz, 1H), 7.35 (d, J=8.82 Hz, 2H), 7.44-7.60 (m, 5H), 7.68 (d, J=7.61 Hz, 2H), 7.88 (m, 2H), 8.62 (d, J=4.68 Hz, 1H), 9.14 (s, 1H, OH), 9.63 (s, 1H, NH).

MH⁺ 440.34.

### EXAMPLE 7

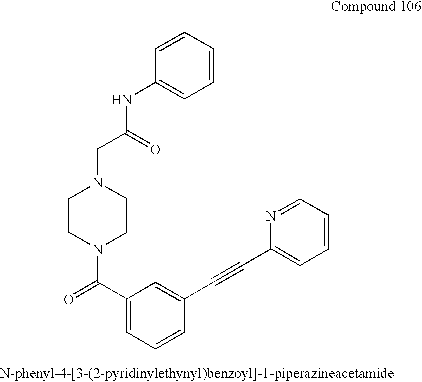

Step A:

To a solution of 3-iodobenzoic acid (7.86 g, 29.5 mmol) in DMF (100 ml) at room temperature was added 1-(ethoxycarbonyl)methylpiperazine (5.08 g, 29.5 mmol), N,N-diisopropylethylamine (DIPEA) (10.3 ml, 59.0 mmol), and o-(7-azabenzotriazol-1-yl)N,N,N′,N′-tetramethyuronium hexafluorophosphate (HATU) (13.46 g, 35.4 mmol). The resultant solution was allowed to stir for 2 days at room temperature, and then water (100 ml) was added to the solution. The solution was extracted with ethyl acetate (3×100 mL). The organic layers were combined, washed with water and dried over MgSO₄. The solution was filtered and the volatiles removed in vacuo. The residue was purified by flash chromatography on 230-400 mesh silica gel, eluting with 4:1 ethyl acetate/hexane, to yield the product as a colorless oil.

Step B:

To a stirring solution of the compound prepared in Step A (8.24 g, 20.5 mmol) in methanol (15 ml) at room temperature, was added a solution of KOH (1.72 g, 30.6 mmol) in water (20 ml). After stirring at room temperature for 1.5 hr, aqueous concentrated HCl (5 ml) was added dropwise. The solvent was removed by rotary evaporation and the residue was dissolved in methanol. The white precipitate was removed by filtration. The filtrate was concentrated to dryness via rotary evaporation to yield the crude product as an HCl salt, a white solid, which was used without further purification.

Step C: (compound #102)

To a solution of the product prepared in Step B at room temperature, was added aniline (2.29 g, 24.6 mmol), N,N-diisopropylethylamine (21 ml, 123 mmol) in DMF (50 ml), 2-(1H-Benzotriazole-1-yl)-1,1,3,3-tetramethyluronium hexafluorophosphate (HBTU) (9.32 g, 24.6 mmol). The resultant solution was allowed to stir overnight at room temperature and then water (50 ml) was added to the solution. Aqueous NaOH solution (3 N) was added dropwise until the solution was slightly basic. The solution was extracted with ethyl acetate (3×50 ml). The combined organic layers were washed with water (50 ml) and dried over MgSO₄. The solution was concentrated and the residue was purified by flash chromatography on 230-400 mesh silica gel, eluting with 4:1 ethyl acetate/hexane, to yield the product as a colorless oil.

Step D:

To a stirring solution of the compound prepared in Step C (1.24 g, 2.76 mmol) in a mixture of solvents DMF (4.0 ml) and triethyl amine (4.0 ml) at room temperature was added 2-ethynylpyridine (0.57 g, 5.53 mmol) and copper(I) iodide (0.052 g, 0.27 mmol). The mixture was degassed by bubbling argon in vigorously for 10 min. Dichlorobis(triphenylphosphine)palladium(II) (0.29 g, 0.41 mmol) was then added. The solution was heated at 118° C. in a pressure tube for 18 hr. The mixture was allowed to warm to room temperature and the volatiles removed by rotary evaporation. The residue was purified by column chromatography over silica gel eluting with ethyl acetate/hexanes (90/10) to yield the product as a slightly colored oil which was converted to an HCl salt by treatment with HCl in ethyl acetate.

¹H NMR (300 MHz, CD₃OD), δ 2.41 (broad, 8H), 3.10 (s, 2H), 5.96 (dd, J=7, 8 Hz, 1H), 6.15 (dd, J=8, 8 Hz, 2H), 6.33-6.55 (m, 4H), 6.70 (d, J=7 Hz, 1H), 6.76 (s, 1H), 6.85 (dd, J=6, 7 Hz, 1H), 7.06 (d, J=8, Hz, 2H), 7.42 (dd, J=7, 8 Hz, 1H), 7.68 (d, J=5 Hz, 1H).

MH⁺ 425.32.

### EXAMPLE 8

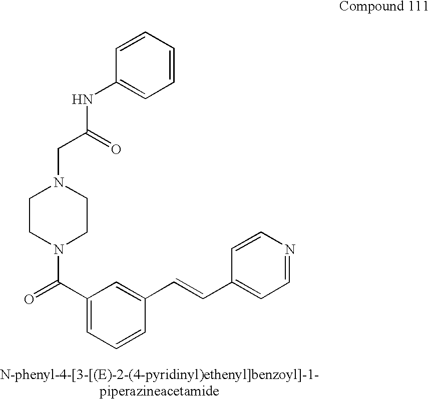

To a solution of the compound as prepared in Step C of Example 7, (0.51 g, 1.13 mmol) in a mixture of solvents DMF (2.0 ml) and triethyl amine (2.0 ml) at room temperature was added 4-ethylenepyridine (0.23 ml, 2.26 mmol). The solution was degassed by bubbling argon in for 10 min. Bis(acetato)bis(triphenylphosphine)palladium(II) (0.017 g, 0.023 mmol) was then added. The solution was heated at 100° C. in a pressure tube for 24 hr. After removing the solvents by rotary evaporation, the residue was purified by column chromatography over silica gel eluting with ethyl acetate to yield the product as a colorless oil which was converted to an HCl salt by treatment with HCl in ethyl acetate.

¹H NMR (300 MHz, CD₃OD), δ 3.59 (broad, 8H), 4.27 (s, 2H), (dd, J=8, 9 Hz, 1H), 7.13 (dd, J=8,9 Hz 1H), 7.33 (dd, J=7, 9 Hz, 2H), 7.56-7.64 (m, 5H), 7.90-8.03 (m, 3H), 8.26 (d, J=7 Hz, 2H), 8.75 (d, J=7 Hz, 2H).

MH⁺ 427.26.

### EXAMPLE 9

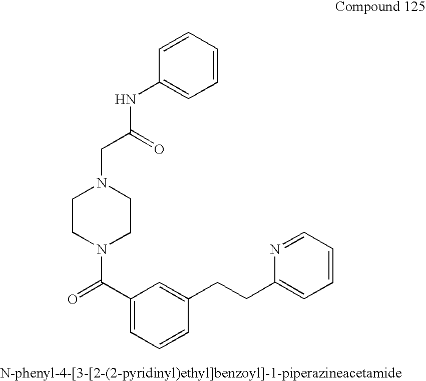

To a solution of the compound prepared as in Example 8 (0.093 g, 0.22 mmol) in ethanol (40 ml) at room temperature was added palladium on carbon (10%, 0.093 g). The resultant mixture was subjected to hydrogen gas at 50 psi overnight. The solution was filtered through Celite and the filtrate concentrated via rotary evaporation. The residue was purified by preparative HPLC to yield the product as a white solid, as a trifluoroacetate salt.

¹H NMR (300 MHz, CD₃OD), δ 3.38 (broad m, 8H), 3.88 (broad, 4H), 4.13(s, 2H), 7.13 (dd, J=7, 7 Hz, 1H), 7.30-7.44 (m, 6H), 7.58 (d, J=8 Hz, 2H), 7.83-7.90 (m, 2H), 8.44 (dd, J=8, 8 Hz, 2H), 8.70 (d, J=6 Hz, 1H).

MH⁺ 429.26.

### EXAMPLE 10

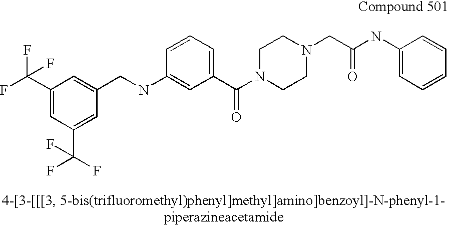

Step A:

Wang p-nitrophenylcarbonate resin (10 g, 6.67 mmol) was swelled in a mixed solvent of DCM (40 mL) and NMP (20 mL). To the suspension were added 3-aminobenzoic ethyl ester (11.05 g, 66.9 mmol), DIPEA (11.65 mL, 66.9 mmol), and HOBT (5.15 g, 33.6 mmol). The mixture was shaken for 16 hours at room temperature. The solvents were removed by filtration, and the resin was washed by DCM and methanol three times alternately. The resin was dried in vacuum for 6 hours.

Step B:

The carbamate resin from A was swelled in NMP (60 mL). To the suspension was added NaH (884 mg, 22.11 mmol). After shaking for 3 hours at room temperature, 3,5-bis(trifluoromethyl)benzyl bromide (6.75 mL, 36.85 mmol) was added to the reaction. The mixture was shaken for 16 hour at room temperature. The solvents were removed by filtration, and the resin was washed by NMP three times, then DCM and methanol three times alternately. The resin was dried in vacuum for 6 hours.

Step C:

The alkylated resin from B was suspended in a mixed solvent 1.0 N NaOH (40 mL) aqueous solution and DME (40 mL). The suspension was shaken for 16 hours at 55° C. The solvents were removed by filtration, and the resin was washed by water three times, then DCM and methanol three times alternately. The resin was dried in vacuum for 6 hours.

Step D:

The benzoic acid resin from C (1.0 g, 0.54 mmol) was swelled in NMP (10 mL). To the suspension were added DIC (0.254 mL, 1.62 mmol), HOBT (248 mg, 1.62 mmol), and 1-(ethoxycarbonylmethyl)piperazine (279 mg, 1.62 mmol). The mixture was shaken for 16 hour at room temperature. The solvents were removed by filtration, and the resin was washed by NMP three times, then DCM and methanol three times alternately. The resin was dried in vacuum for 6 hours.

Step E:

The substituted acetic ethyl ester resin from D was suspended in a mixed solvent of 1.0 N NaOH (5 mL) aqueous solution and DME (5 mL). The suspension was shaken for 16 hours at 55° C. The solvents were removed by filtration, and the resin was washed by water three times, then DCM and methanol three times alternately. The resin was dried in vacuum for 6 hours.

Step F:

The acetic acid resin from Step E was divided into four portions each containing 0.135 mmol of resin. One portion was swelled in NMP (2 mL). To the suspension were added aniline (0.0615 mL, 0.675 mmol), HATU (1.03 g, 0.675 mmol), and DIPEA (0.47 mL, 0.675 mmol). The suspension was shaken for 16 hours at room temperature. The solvents were removed by filtration, and the resin was washed by NMP three times, then DCM and methanol three times alternately. The resin was dried in vacuum for 6 hours.

Step G:

The resin from Step F was treated with a cleaving cocktail solution of 50:50 TFA:DCM and the cleavage solution was evaporated to cleaved the product from the resin. The product was purified by semi-preparative reversed phase HPLC on a 20×100 mm J'sphere H-80 YMC column using a gradient of 90:10:0.1 water:acetonitrile:TFA to 10:90:0.1 water:acetonitrile:TFA. The product was speed-vacuum dried and analyzed by ES+/MS/reversed phase HPLC.

MH⁺ 565.3

Compound 505 (RWJ-406275-279) was similarly prepared according the above procedure, using 1-(ethoxycarbonylmethyl)piperidine in step D and appropriate selection and substitution of a suitably substituted amines in Step F.

### EXAMPLE 11

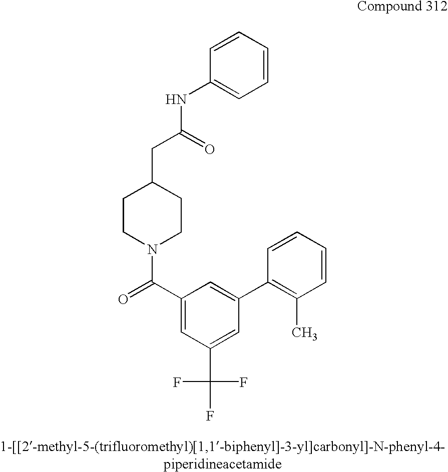

Step 1:

FMPB resin (120 mg, 0.12 mmol) [purchased from Irori] was placed in a 3 ml polypropylene tube and washed with DMF (2×1 ml). The resin was suspended in DMF (0.5 ml) and trimethyl orthoformate (0.5 ml), aniline (0.056 ml, 0.61 mmol), acetic acid (20 μl), and sodium triacetoxyborohydride (129 mg, 0.61 mmol) were added. The resulting slurry was agitated for 18 h at room temperature. The resin was filtered and washed with DCM (2×1 ml), methanol (2×1 ml), water (2×1 ml), methanol (2×1 ml), DCM (1 ml), methanol (1 ml), DCM (1 ml), methanol (1 ml), DCM (4×1 ml).

Step 2:

The resin from Step 1 was suspended in DCM (1.2 ml) and Fmoc-(4-carboxymethyl)-piperidine (90 mg, 0.25 mmol) [purchased from Neosystem] and DIPEA (0.13 ml, 0.73 mmol) were added. The resulting slurry was agitated for 1 minute. 2-chloro-1,3-dimethylimidazolium chloride (62 mg, 0.37 mmol) was then added in one portion. The solution was shaken for 18 h at room temperature. The resin was filtered and washed with DCM (2×1 ml), methanol (1 ml), DCM (1 ml), methanol (1 ml), DCM (1 ml), methanol (1 ml), DCM (4×1 ml). The Fmoc protecting group was removed with 25% piperidine in DMF (2×1 ml) for 30 minutes each. The resin was filtered and washed with DCM (2×1 ml), methanol (1 ml), DCM (1 ml), methanol (1 ml), DCM (1 ml), methanol (1 ml), DCM (4×1 ml).

Step 3:

The resin from Step 2 was suspended in DCM (1.2 ml). 3-Bromo-5-trifluoromethyl benzoic acid (66 mg, 0.25 mmol) and DIPEA (0.13 ml, 0.73 mmol) were added. The resulting slurry was agitated for 1 minute. 2-chloro-1,3-dimethylimidazolium chloride (62 mg, 0.37 mmol) was then added in one portion. The solution was shaken for 18 h at room temperature. The resin was filtered and washed with DCM (2×1 ml), methanol (1 ml), DCM (1 ml), methanol (1 ml), DCM (1 ml), methanol (1 ml), DCM (2×1 ml), and DMF (2×1 ml).

Step 4:

The resin from Step 3 was placed in a glass reactor and suspended in DMF (1 ml). Nitrogen was bubbled through the solution for 5 minutes. To the bubbling solution was added o-tolylboronic acid (166 mg, 1.2 mmol), potassium carbonate (203 mg, 1.5 mmol) in water (200 μl), and tetrakis(triphenylphosphine) palladium(0) (15 mg, 0.012 mmol). The resulting slurry was agitated and heated to 80° C. in a sealed tube for 18 h.

The product was cleaved from the resin using a solution of 50:50 TFA:DCM. The cleavage solution was evaporated and the product was purified by semi-preparative reversed phase HPLC on a 20×100 mm J'sphere H-80 YMC column using a gradient of 100:0.1 water:TFA to 5:95:0.1 water:acetonitrile:TFA. The eluent containing was evaporated to yield the product as a white solid.

MS detected [M⁺¹]: 481.2.

Compound 316 was similarly prepared according the above procedure with appropriate selection of reagents for Step 4 above.

### Example 12

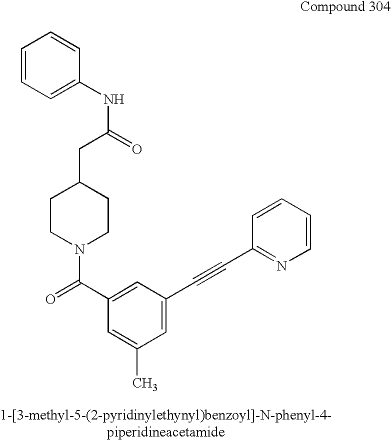

The resin prepared in Step 2 in Example 11 above was placed in a glass reactor and suspended in DCM (1.2 ml). 3-Bromo-5-methyl benzoic acid (54 mg, 0.25 mmol) and DIPEA (0.13 ml, 0.73 mmol) were added. The resulting slurry was agitated for 1 minute. 2-chloro-1,3-dimethylimidazolium chloride (62 mg, 0.37 mmol) was then added in one portion. The solution was shaken for 18 h at room temperature. The resin was filtered and washed with DCM (2×1 ml), methanol (1 ml), DCM (1 ml), methanol (1 ml), DCM (1 ml), methanol (1 ml), DCM (2×1 ml), and DMF (2×1 ml).

The resin was suspended in DMF (1 ml). Nitrogen was bubbled through the solution for 5 minutes. To the bubbling solution was added 2-ethynylpyridine (124 mg, 1.2 mmol), triethylamine (50 μl), tri-o-tolylphosphine (20 mg), copper(I) iodide (2.3 mg), and palladium(II) acetate (20 mg). The resulting slurry was agitated and heated to 80° C. in a sealed tube for 18 h.

The product was cleaved from the resin using a solution of 50:50 TFA:DCM. The cleavage solution was evaporated and the product was purified by semi-preparative reversed phase HPLC on a 20×100 mm J'sphere H-80 YMC column using a gradient of 100:0.1 water:TFA to 5:95:0.1 water:acetonitrile:TFA. The eluent was evaporated to yield the product as a white solid.

MS detected [M⁺¹]: 438.3.

Compound 306 was similarly prepared according the above procedure with appropriate selection of reagents.

Following the procedures described above, specific compounds of the instant invention were prepared, as listed in Tables 1-10, below.

TABLE 1

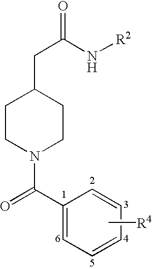

| ID # | R² | R⁴ | Calc. MW | Meas. MW |
|---|---|---|---|---|
| 1 | —CH₂-(3- | 3-Phenyl | 480.53 | 481.23 |
| | trifluoromethylphenyl) | | | |
| 2 | —CH₂-cyclohexyl | 3-Phenyl | 418.58 | 419.31 |
| 3 | —CH₂-(3,5- | 3-Phenyl | 472.58 | 473.25 |
| | dimethoxyphenyl) | | | |
| 4 | —CH₂-(4- | 3-Phenyl | 480.53 | 481.21 |
| | trifluoromethylphenyl) | | | |
| 5 | —CH₂-(3,5- | 3-Phenyl | 548.52 | 549.25 |
| | ditrifluoromethylphenyl) | | | |
| 6 | 3-trifluoromethoxyphenyl | 3-Phenyl | 482.50 | 483.20 |
| 7 | —CH₂-(4-dimethyl- | 3-Phenyl | 455.60 | 456.28 |
| | aminophenyl) | | | |
| 8 | Phenyl | 3-Phenyl | 398.50 | 399.23 |

TABLE 2

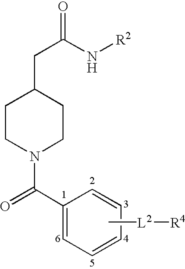

| | | | | Calc. | Meas. |
|---|---|---|---|---|---|
| ID # | R² | L² | R⁴ | MW | MW |
| 9 | Phenyl | 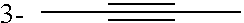 | 3-Phenyl | 422.52 | 423.00 |
| 10 | Phenyl | 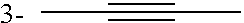 | 2-Pyridyl | 423.51 | 424.38 |
| 11 | —CH₂-(4-dimethyl aminophenyl) | 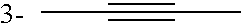 | Phenyl | 479.62 | 480.24 |
| 12 | —CH₂-(4-trifluoro methylphenyl) | 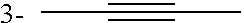 | Phenyl | 504.55 | 505.41 |
| 13 | Benzyl | 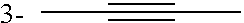 | Phenyl | 436.55 | 437.40 |
| 14 | 4-fluorophenyl | 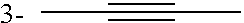 | 2-Pyridyl | 441.50 | 442.25 |
| 15 | 2,4- difluorophenyl | 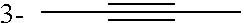 | 2-Pyridyl | 459.49 | 460.22 |
| 16 | 2-fluorophenyl | 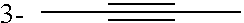 | 2-Pyridyl | 441.50 | 442.24 |
| 17 | 2,6- difluorophenyl | 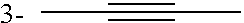 | 2-Pyridyl | 459.49 | 460.23 |
| 18 | Phenyl | 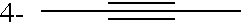 | 3-Pyridyl | 423.51 | 424.25 |
| 19 | 4-fluorophenyl | 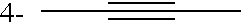 | 3-Pyridyl | 441.50 | 442.26 |
| 20 | 2-fluorophenyl | 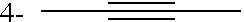 | 3-Pyridyl | 441.50 | 442.23 |
| 21 | 2,4- difluorophenyl | 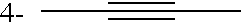 | 3-Pyridyl | 459.49 | 460.25 |
| 22 | 2,6- difluorophenyl | 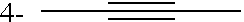 | 3-Pyridyl | 459.49 | 460.21 |
| 23 | Phenyl | 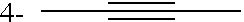 | 2-Pyridyl | 423.51 | 424.25 |
| 24 | 4-fluorophenyl | 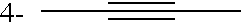 | 2-Pyridyl | 441.50 | 442.23 |
| 25 | 2-fluorophenyl | 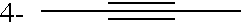 | 2-Pyridyl | 441.50 | 442.31 |
| 26 | 2,4- difluorophenyl | 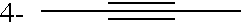 | 2-Pyridyl | 459.49 | 460.25 |
| 27 | 2,6- difluorophenyl | 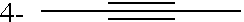 | 2-Pyridyl | 459.49 | 460.24 |
| 28 | Phenyl | 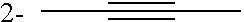 | 2-Pyridyl | 423.51 | 424.30 |
| 29 | 4-fluorophenyl | 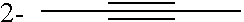 | 2-Pyridyl | 441.50 | 442.27 |
| 30 | 2-fluorophenyl | 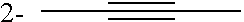 | 2-Pyridyl | 441.50 | 442.25 |
| 31 | 2,4- difluorophenyl | 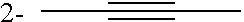 | 2-Pyridyl | 459.49 | 460.24 |
| 32 | 2,6- difluorophenyl | 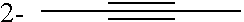 | 2-Pyridyl | 459.49 | 460.21 |
| 33 | 2,4- difluorophenyl | 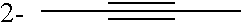 | 4-Pyridyl | 459.49 | 460.29 |
| 34 | 2-fluorophenyl | 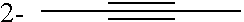 | 4-Pyridyl | 441.50 | 442.31 |
| 35 | 4-fluorophenyl | 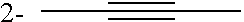 | 4-Pyridyl | 441.50 | 442.23 |
| 36 | Phenyl | 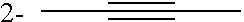 | 4-Pyridyl | 423.51 | 424.30 |
| 37 | Phenyl | 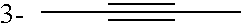 | 3-Pyridyl | 423.51 | 424.27 |
| 38 | 2-fluorophenyl | 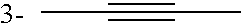 | 3-Pyridyl | 441.50 | 442.25 |
| 39 | 4-fluorophenyl | 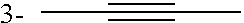 | 3-Pyridyl | 441.50 | 442.18 |
| 40 | 2,4- difluorophenyl | 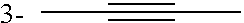 | 3-Pyridyl | 459.49 | 460.26 |
| 41 | 2,6- difluorophenyl | 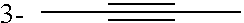 | 3-Pyridyl | 459.49 | 460.23 |
| 42 | Phenyl | 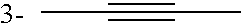 | 4-Pyridyl | 423.51 | 424.30 |
| 43 | 2-fluorophenyl | 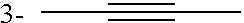 | 4-Pyridyl | 441.50 | 442.29 |
| 44 | 4-fluorophenyl | 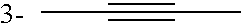 | 4-Pyridyl | 441.50 | 442.27 |
| 45 | 2,4- difluorophenyl | 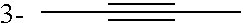 | 4-Pyridyl | 459.49 | 460.28 |
| 46 | 2,6- difluorophenyl | 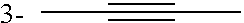 | 4-Pyridyl | 459.49 | 460.27 |
| 57 | Phenyl | 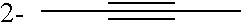 | 3-pyridyl | 423.51 | 424.28 |
| 58 | 2-fluorophenyl | 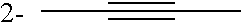 | 3-Pyridyl | 441.50 | 442.26 |
| 59 | 4-fluorophenyl | 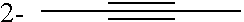 | 3-Pyridyl | 441.50 | 442.26 |
| 60 | 2,4- difluorophenyl | 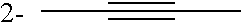 | 3-Pyridyl | 459.49 | 460.24 |
| 61 | Phenyl | 3-CH₂—CH₂— | 4-Pyridyl | 427.54 | 428.29 |
| 62 | 4-fluorophenyl | 3-CH₂—CH₂— | 4-Pyridyl | 445.53 | 446.29 |
| 63 | Phenyl | 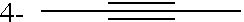 | 4-Pyridyl | 423.51 | 424.24 |
| 64 | 2-fluorophenyl | 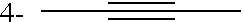 | 4-Pyridyl | 441.50 | 442.24 |
| 65 | 4-fluorophenyl | 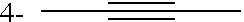 | 4-Pyridyl | 441.50 | 442.25 |
| 66 | 2,6- difluorophenyl | 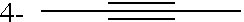 | 4-Pyridyl | 459.49 | 460.25 |
| 67 | Phenyl | 4-CH₂—CH₂— | 4-Pyridyl | 427.54 | 428.30 |
| 68 | 2-fluorophenyl | 4-CH₂—CH₂— | 4-Pyridyl | 445.53 | 446.28 |
| 69 | 4-fluorophenyl | 4-CH₂—CH₂— | 4-Pyridyl | 445.53 | 446.29 |
| 70 | 2,4- | 4-CH₂—CH₂‘3 | 4-Pyridyl | 463.25 | 464.27 |
| | difluorophenyl | | | | |
| 71 | 2,6- | 4-CH₂—CH₂— | 4-Pyridyl | 463.52 | 464.26 |
| | difluorophenyl | | | | |
| 72 | phenyl | 3-CH₂—CH₂— | 2-Pyridyl | 427.54 | 428.33 |
| 73 | phenyl | 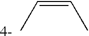 | 4-Pyridyl | 425.53 | 426.27 |
| 74 | phenyl | 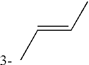 | 2-Pyridyl | 425.53 | 426.30 |
| 75 | 4-hydroxyphenyl | 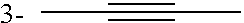 | 2-Pyridyl | 439.51 | 440.34 |
| 76 | 2-fluorophenyl | 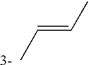 | 4-pyridyl | 443.52 | |
| 77 | 4-fluorophenyl | 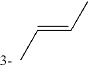 | 4-pyridyl | 443.52 | |
| 78 | 2,4-difluoro phenyl | 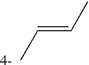 | 4-pyridyl | 431.51 | |
| 79 | 2-fluorophenyl | 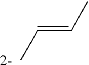 | 2-pyridyl | 443.52 | |
| 80 | phenyl | 4-(CH₂—N(CH₃)—(CH₂CH₂)— | 1-pyrrolidinyl | 462.63 | |
| 81 | phenyl | 4-(CH₂—N(CH₃)—(CH₂)— | 2-furyl | 445.56 | |
| 82 | phenyl | 4-(CH₂—N(CH₃)—CH₂)— | 1-naphthyl | 505.66 | |
| 83 | phenyl | 4-(CH₂—N(C(O)CH₃)—CH₂)— | 2-pyridyl | 484.60 | |
| 401 | 4-hydroxyphenyl | 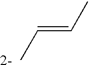 | 2-pyridyl | 441.53 | |
| 402 | phenyl | 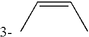 | 2-pyridyl | 425.53 | |
| 403 | 2-fluorophenyl | 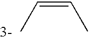 | 2-pyridyl | 443.52 | |
| 404 | 4-fluorophenyl | 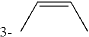 | 2-pyridyl | 443.52 | |
| 405 | 2,6-difluoro phenyl | 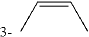 | 2-pyridyl | 461.51 | |
| 406 | 4-hydroxyphenyl | 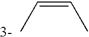 | 2-pyridyl | 441.53 | |
| 407 | 4-methoxy phenyl | 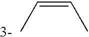 | 2-pyridyl | 455.56 | |
| 409 | phenyl | 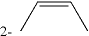 | 2-pyridyl | 425.53 | |
| 410 | 2-fluorophenyl | 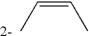 | 2-pyridyl | 443.52 | |
| 411 | 2,6-difluoro phenyl | 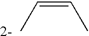 | 2-pyridyl | 461.51 | |
| 412 | 4-hydroxyphenyl | 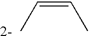 | 2-pyridyl | 441.53 | |
| 413 | 4-methoxy phenyl | 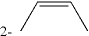 | 2-pyridyl | 455.56 | |
| 414 | phenyl | 2-CH₂CH₂ | 2-pyridyl | 427.55 | |
| 415 | 2-fluorophenyl | 2-CH₂CH₂ | 2-pyridyl | 445.54 | |
| 416 | 4-fluorophenyl | 2-CH₂CH₂ | 2-pyridyl | 445.54 | |
| 417 | 2,4-difluoro | 2-HC₂CH₂ | 2-pyridyl | 463.53 | |
| | phenyl | | | | |
| 418 | 4-hydroxyphenyl | 2-CH₂CH₂ | 2-pyridyl | 443.54 | |
| 419 | 4-methoxyphenyl | 2-CH₂CH₂ | 2-pyridyl | 457.57 | |
| 429 | 2-fluorophenyl | 3-CH₂CH₂ | 2-pyridyl | 445.54 | |
| 430 | 4-fluorophenyl | 3-CH₂CH₂ | 2-pyridyl | 445.54 | |
| 431 | 2,4-difluoro | 3-CH₂CH₂ | 2-pyridyl | 463.53 | |
| | phenyl | | | | |
| 432 | 2,6-difluoro | 3-CH₂CH₂ | 2-pyridyl | 463.53 | |
| | phenyl | | | | |
| 433 | 4-hydroxyphenyl | 3-CH₂CH₂ | 2-pyridyl | 443.54 | |
| 434 | 4-methoxy | 3-CH₂CH₂ | 2-pyridyl | 457.57 | |
| 435 | 4-dimethyl | 3-CH₂CH₂ | 2-pyridyl | 470.61 | |
| | aminophenyl | | | | |
| 436 | 4-trifluoro | 3-CH₂CH₂ | 2-pyridyl | 495.53 | |
| | methylphenyl | | | | |
| 437 | phenyl | 3-CH₂CH₂ | 2-pyridyl | 427.55 | |
| 438 | 2-fluorophenyl | 4-CH₂CH₂ | 2-pyridyl | 445.54 | |
| 439 | 4-fluorophenyl | 4-CH₂CH₂ | 2-pyridyl | 445.54 | |
| 440 | 2,4-difluoro | 4-CH₂CH₂ | 2-pyridyl | 463.53 | |
| | phenyl | | | | |
| 441 | 2,6-difluoro | 4-CH₂CH₂ | 2-pyridyl | 463.53 | |
| | phenyl | | | | |
| 442 | 4-hydroxyphenyl | 4-CH₂CH₂ | 2-pyridyl | 443.54 | |
| 443 | 4-methoxy | 4-CH₂CH₂ | 2-pyridyl | 457.57 | |
| | phenyl | | | | |
| 444 | 4-dimethyl | 4-CH₂CH₂ | 2-pyridyl | 470.61 | |
| | aminophenyl | | | | |
| 445 | 4-trifluoro | 4-CH₂CH₂ | 2-pyridyl | 495.54 | |
| | methylphenyl | | | | |
| 457 | 3-pyridyl | 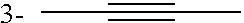 | 2-pyridyl | 424.50 | |
| 458 | 4-pyridyl | 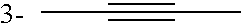 | 2-pyridyl | 424.50 | |
| 460 | 4-pyrimidinyl | 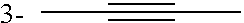 | 2-pyridyl | 425.49 | |
| 461 | 2-pyridyl | 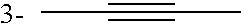 | 2-pyridyl | 424.50 | |
| 462 | 2-pyrimidinyl | 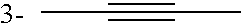 | 2-pyridyl | 425.49 | |
| 463 | phenyl | 4-CH₂—N(CH₃)—CH₂CH₂ | 1-pyrrolidinyl | 462.63 | |
| 464 | phenyl | 4-CH₂—N(CH₃)—CH₂ | 2-furyl | 445.56 | |
| 465 | phenyl | 4-CH₂—N(CH₃)—CH₂ | 1-naphthyl | 505.66 | |
| 466 | phenyl | 4-CH₂—N(CH₃)—CH₂ | 2-thienyl | 461.63 | |
| 467 | phenyl | 4-CH₂—N(CH₃)—CH₂ | 2-pyridyl | 456.59 | |
| 468 | phenyl | 4-CH₂—N(CH₃)—CH₂ | 2-benzimi- | 495.62 | |
| | | | dazolyl | | |
| 469 | phenyl | 4-CH₂—N(CH₃)—CH₂ | 2R-tetrahydro | 449.59 | |
| | | | furyl | | |
| 470 | phenyl | 4-CH₂—N(CH₃)—CH₂CH₂ | 1-imidazolyl | 459.59 | |
| 471 | phenyl | 4-CH₂—N(C(O)CH₃)—CH₂CH₂ | 1-pyrrolidinyl | 490.64 | |
| 472 | phenyl | 4-HC₂—N(C(O)—CH₃)—CH₂ | 2-furyl | 473.57 | |
| 473 | phenyl | 4-CH₂—N(C(O)—CH₃)—CH₂ | 1-naphthyl | 533.67 | |
| 474 | phenyl | 4-CH₂—N(C(O)—CH₃)—CH₂ | 2-thienyl | 489.64 | |
| 475 | phenyl | 4-CH₂—N(C(O)—CH₃)—CH₂ | 2-pyridyl | 484.60 | |
| 476 | phenyl | 4-CH₂—N(C(O)—CH₃)—CH₂ | 2-benzimi- | 523.63 | |
| | | | sazolyl | | |
| 477 | phenyl | 4-CH₂—N(C(O)—CH₃)—CH₂ | 2R-tetra | 477.60 | |
| | | | hydrofuryl | | |
| 478 | phenyl | 4-CH₂—N(C(O)—CH₃)—CH₂CH₂ | 1-imidazolyl | 487.60 | |

TABLE 3

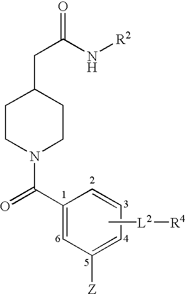

| ID # | R² | L² | R⁴ | Z | Calc. MW | Meas. MW |
|---|---|---|---|---|---|---|
| 301 | Phenyl | 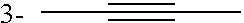 | Phenyl | —CF₃ | 490.2 | 491.2 |
| 302 | Phenyl | 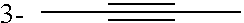 | Phenyl | —CH₃ | 436.2 | 437.3 |
| 303 | Phenyl | 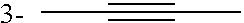 | 2-Pyridyl | —CF₃ | 491.2 | 492.2 |
| 304 | Phenyl | 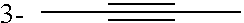 | 2-Pyridyl | —CH₃ | 437.2 | 438.3 |
| 305 | Phenyl | 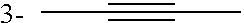 | 3-Pyridyl | —CF₃ | 491.2 | 492.3 |
| 306 | Phenyl | 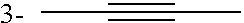 | 3-Pyridyl | —CH₃ | 437.2 | 438.3 |
| 307 | Phenyl | 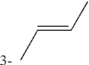 | 4-Pyridyl | —CF₃ | 493.3 | 494.2 |
| 308 | Phenyl | 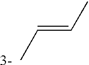 | 2-Pyridyl | —CF₃ | 493.2 | 494.2 |
| 309 | Phenyl | 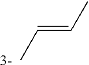 | 2-Pyridyl | —CH₃ | 439.2 | 440.3 |
| 310 | Phenyl | 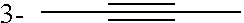 | 3-Hydroxy phenyl | —CF₃ | 506.2 | 507.2 |
| 311 | Phenyl | 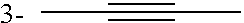 | 3-Hydroxy phenyl | —CH₃ | 452.2 | 453.3 |
| 479 | 2,4- difluoro phenyl | 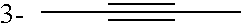 | 2-pyridyl | CH₃ | 473.52 | |
| 480 | 2,4- difluoro phenyl | 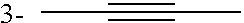 | 2-pyridyl | CF₃ | 527.49 | |

TABLE 4

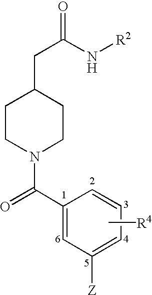

| ID # | R² | R⁴ | Z | Calc. MW | Mass. MW |
|---|---|---|---|---|---|
| 312 | Phenyl | 3-(2-methylphenyl) | —CF₃ | 480.2 | 481.2 |
| 313 | Phenyl | 3-(2-methylphenyl) | —CH₃ | 426.2 | 427.3 |
| 314 | Phenyl | 3-phenyl | —CF₃ | 466.2 | 467.2 |
| 315 | Phenyl | 3-phenyl | —CH₃ | 412.2 | 413.3 |
| 316 | Phenyl | 3-(3-aminophenyl) | —CF₃ | 481.2 | 482.2 |
| 317 | Phenyl | 3-(3-aminophenyl) | —CH₃ | 427.2 | 428.3 |
| 318 | Phenyl | 3-(3-pyridyl) | —CF₃ | 467.2 | 468.3 |
| 319 | Phenyl | 3-(3-pyridyl) | —CH₃ | 413.2 | 414.3 |
| 320 | Phenyl | 3-(3-thienyl) | —CF₃ | 472.1 | 473.2 |
| 321 | Phenyl | 3-(3-thienyl) | —CH₃ | 418.2 | 419.2 |

TABLE 5

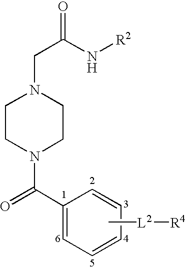

| ID # | R² | L² | R⁴ | Calc. MW | Meas. MW |
|---|---|---|---|---|---|
| 103 | Phenyl | 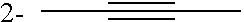 | 2-Pyridyl | 424.50 | 425.23 |
| 104 | Phenyl | 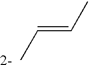 | 2-Pyridyl | 426.52 | 427.25 |
| 105 | Phenyl | 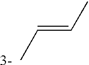 | 2-Pyridyl | 426.52 | 427.25 |
| 106 | Phenyl | 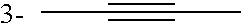 | 2-Pyridyl | 424.50 | 425.25 |
| 107 | Phenyl | 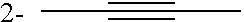 | 3-Pyridyl | 424.50 | 425.26 |
| 108 | Phenyl | 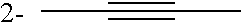 | 4-Pyridyl | 424.50 | 425.24 |
| 109 | Phenyl | 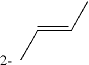 | 4-Pyridyl | 424.50 | 425.32 |
| 110 | Phenyl | 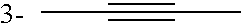 | 3-Pyridyl | 426.52 | 427.26 |
| 111 | Phenyl | 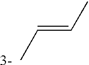 | 4-Pyridyl | 426.52 | 427.26 |
| 112 | Phenyl | 3-CH₂—CH₂— | 2-Pyridyl | 428.53 | 429.27 |
| 113 | Phenyl | 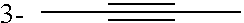 | Phenyl | 423.51 | 424.23 |
| 114 | 4-fluoro phenyl | 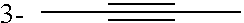 | 2-Pyridyl | 442.49 | 443.26 |
| 115 | 2,4-difluoro phenyl | 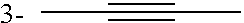 | 2-Pyridyl | 460.48 | 461.23 |
| 116 | 2-fluoro phenyl | 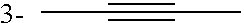 | 2-Pyridyl | 442.49 | 443.25 |
| 117 | 2,4-difluoro phenyl | 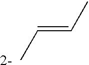 | 2-Pyridyl | 444.51 | 445.63 |
| 118 | 2-fluoro phenyl | 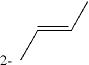 | 2-Pyridyl | 444.51 | 445.63 |
| 119 | 2,4-difluoro phenyl | 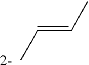 | 2-Pyridyl | 462.509 | 463.34 |
| 120 | 4-fluoro phenyl | 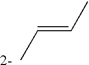 | 2-Pyridyl | 444.51 | 445.34 |
| 121 | 2-fluoro phenyl | 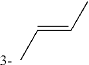 | 2-Pyridyl | 444.51 | 445.35 |
| 122 | 4-fluoro phenyl | 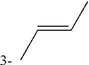 | 2-Pyridyl | 444.51 | 445.34 |
| 123 | 2,4-difluoro phenyl | 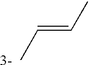 | 2-Pyridyl | 462.50 | 463.33 |
| 124 | 2,6-difluoro phenyl | 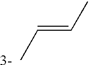 | 2-Pyridyl | 462.48 | 463.24 |
| 125 | Phenyl | 3-CH₂—CH₂— | 2-Pyridyl | 428.53 | 429.28 |
| 126 | 4-fluoro phenyl | 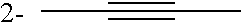 | 2-Pyridyl | 442.49 | 443.3 |
| 127 | 2,4-difluoro phenyl | 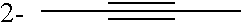 | 2-Pyridyl | 460.48 | 461.29 |
| 128 | 2-fluoro phenyl | 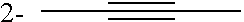 | 2-Pyridyl | 442.49 | 443.3 |
| 129 | 2,6-difluoro phenyl | 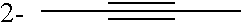 | 2-Pyridyl | 460.48 | 461.28 |
| 137 | CH(CH₃)₂ | 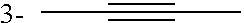 | 2-Pyridyl | 390.48 | |
| 138 | 1-pyrro- lidinyl | 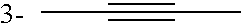 | 2-Pyridyl | 402.50 | |

TABLE 6

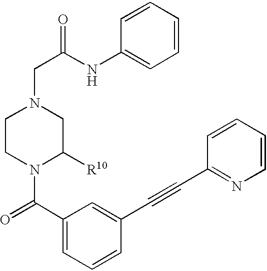

| ID # | R¹⁰ | Calc. MW | Meas. MW |
|---|---|---|---|
| 201 | (R)-methyl | 438.21 | 439.30 |
| 202 | (S)-benzyl | 514.24 | 515.37 |
| 203 | (R)-benzyl | 514.24 | 515.37 |

TABLE 7

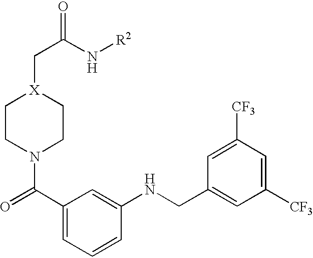

| ID # | X | R² | Calc. MW | Meas. MW |
|---|---|---|---|---|
| 501 | N | phenyl | 564.53 | 565.3 |
| 502 | N | 3-pyridyl | 565.52 | 566.3 |
| 503 | N | 4-(dimethylamino)-phenyl | 607.60 | 608.4 |
| 504 | N | 4-morpholinyl-phenyl | 649.63 | 650.3 |
| 505 | CH | phenyl | 563.55 | 564.4 |
| 506 | CH | 3-pyridyl | 564.54 | 565.4 |
| 507 | CH | 4-(dimethylamino)-phenyl | 606.62 | 607.3 |
| 508 | CH | 4-morpholinyl-phenyl | 648.65 | 649.3 |
| 509 | CH | 4-piperidinyl-phenyl | 646.68 | 647.3 |

TABLE 8

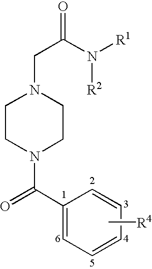

| ID # | R¹ + R² (with the N) | R⁴ | Calc. MW |
|---|---|---|---|
| 130 | 1-pyrrolidinyl | 3-(4-methoxyphenyl) | 407.51 |
| 131 | 1-pyrrolidinyl | 3-(4-chlorophenyl) | 411.93 |
| 132 | 1-piperidinyl | 3-(4-methoxyphenyl) | 421.54 |
| 134 | 1-morpholinyl | 3-(4-methoxyphenyl) | 423.51 |
| 135 | 1-pyrrolidinyl | 3-(4-chlorophenyl) | 413.95 |
| 136 | 1-pyrrolidinyl | 3-(4-methoxyphenyl) | 407.51 |

TABLE 9

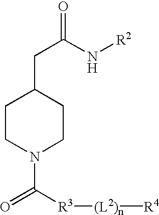

| | | | | | | Calc. |
|---|---|---|---|---|---|---|
| ID # | R² | R³ | n | (L²)ₙ | R⁴ | MW |
| 421 | phenyl | 4-oxazolyl | 0 | absent | 5-phenyl | 389.45 |
| 422 | 2-fluoro | 4-oxazolyl | 0 | absent | 5-phenyl | 407.44 |
| | phenyl | | | | | |
| 423 | 2,4-difluoro | 4-oxazolyl | 0 | absent | 5-phenyl | 425.43 |
| | phenyl | | | | | |
| 424 | 4-fluoro | 4-oxazolyl | 0 | absent | 5-phenyl | 407.44 |
| | phenyl | | | | | |
| 425 | 4-hydroxy | 4-oxazolyl | 0 | absent | 5-phenyl | 405.45 |
| | phenyl | | | | | |
| 426 | 4-methoxy | 4-oxazolyl | 0 | absent | 5-phenyl | 419.48 |
| | phenyl | | | | | |
| 427 | 4-dimethyl | 4-oxazolyl | 0 | absent | 5-phenyl | 432.52 |
| | aminophenyl | | | | | |
| 428 | 4-trifluoro | 4-oxazolyl | 0 | absent | 5-phenyl | 457.45 |
| | methylphenyl | | | | | |
| 446 | phenyl | 4-oxazolyl | 1 | 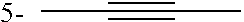 | phenyl | 413.48 |
| 447 | 2-fluoro phenyl | 4-oxazolyl | 1 | 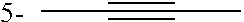 | phenyl | 431.47 |
| 448 | 4-fluoro phenyl | 4-oxazolyl | 1 | 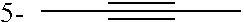 | phenyl | 431.47 |
| 449 | 2,4-difluoro phenyl | 4-oxazolyl | 1 | 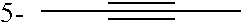 | phenyl | 449.46 |
| 450 | 2-pyridyl | 4-oxazolyl | 1 | 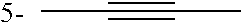 | phenyl | 414.46 |
| 451 | 4-pyridyl | 4-oxazolyl | 1 | 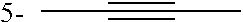 | phenyl | 414.46 |
| 452 | 5-quinolinyl | 4-oxazolyl | 1 | 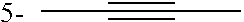 | phenyl | 464.52 |
| 453 | 6-quinolinyl | 4-oxazolyl | 1 | 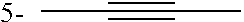 | phenyl | 464.52 |
| 454 | 8-quinolinyl | 4-oxazolyl | 1 | 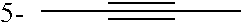 | phenyl | 464.52 |
| 455 | 4-pyridyl methyl | 4-oxazolyl | 1 | 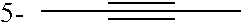 | phenyl | 428.49 |
| 456 | 4-trifluoro methylphenyl | 4-oxazolyl | 1 | 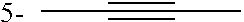 | phenyl | 481.72 |

TABLE 10

| ID# | Structure | Calc. MW |
|---|---|---|
| 133 | 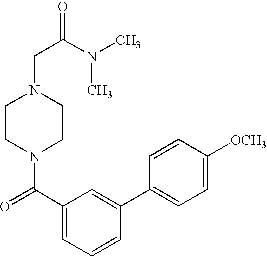 | 381.47 |
| 139 | 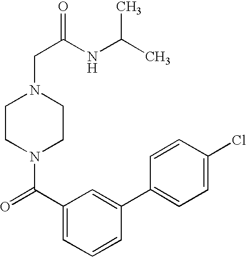 | 399.91 |
| 408 | 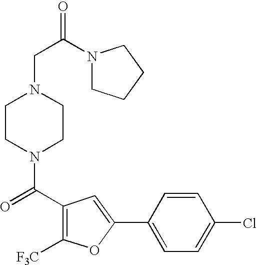 | 469.89 |
| 420 | 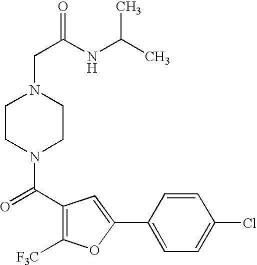 | 457.88 |
| 459 | 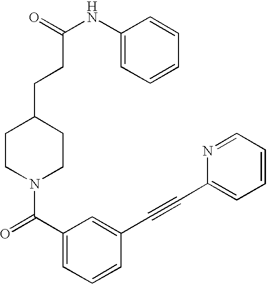 | 437.54 |

### EXAMPLE 13

### In vivo Testing—Doi Headshake Model

Male CD-1 or NIH-Swiss mice were fasted overnight. The mice were given control vehicle or test compound by the oral or intraperitoneal (i.p.) routes of administration at doses up to 40 mg/kg orally and up to 100 mg/kg i.p. Administration time was denoted as to. At each of several selected intervals after to (at about 45 min, 1 h, 2 h, 4 h, 6 h, 8 h, 24 h after administration), separate groups of mice were given 1-{2,5-dimethoxy-4-iodophenyl]-2-aminopropane (DOI), a known serotonin receptor type-2A agonist, by the intraperitoneal route of administration route. After administration of DOI, the mice were observed for 15 min and the number of headshakes induced by the serotonin agonist was measured for mice given the control and mice given the test compound at the above mentioned selected intervals. (Separate groups of mice were tested at each time interval.) Peak activity time, denoted as tₚ, was determined as the time of the greatest reduction in the number of DOI-induced headshakes for mice given the test compound compared to the number of headshakes for the mice given the control, measured at the same time interval.

A statistically significant decrease in the number of headshakes induced by the administration of DOI in the mice given the test compound relative to the mice given the control was an indication of modulation of the serotonin neural pathways and thus an indication of an active compound.

In vivo biological activity was measured for select compounds of the present invention as listed in Table 11, using the procedures outlined above. Starred (*) compounds were tested on both male CD-1 mice and NIH Swiss mice, all other compounds were tested using Swiss NIH mice.

TABLE 11

Number of Headshakes

| ID # | IP Admin | Oral Admin. |
|---|---|---|
| 10* | Active | Active |
| 11 | | Inactive |
| 13* | Inactive | |
| 15 | Active | Active |
| 73 | Active | Active |
| 75 | Active | Active |
| 76 | | Active |
| 77 | Active | Active |
| 78 | Active | Active |
| 79 | Active | Active |
| 80 | Active | Active |
| 81 | | Active |
| 82 | Active | Active |
| 83 | Inactive | |
| 104 | Active | Active |
| 106 | Active | Active |
| 130 | Inactive | |
| 501 | | Inactive |
| 502 | Active | Inactive |

### EXAMPLE 14

### Reversal of Senkide-induced Head Shake in Mice

The in vivo assay measuring the reversal of Senktide-induced headshake in mice has been previously described in the literature by Sarau, H. M., et al in J. Pharmacol. Exp. Therapeutics (2000), 295 pp 373-381.

Briefly, overnight fasted NIH-Swiss mice weighing 18-21 gms were treated with test compound or vehicle by the oral (gavage) route, at various concentrations. Forty five (45) minutes after administration, the animals are injected subcutaneously (sc) with Senktide at a concentration of 5 mg/kg. Immediately after administration of the Senktide, the animals are randomized and laced into isolated observation chambers and the number of headshakes per recorded over a ten (10) minute period. A decrease in the number of Senktide induced headshakes for test compound treated animals as compared with vehicle treated animals(analysis completed using Mann-Whitney t-test (one tailed)) was taken as an indication of anxiolytic activity for the compound.

Representative compounds of the instant invention were tested for reversal of Senktide-induced headshakes in mice, with results as listed in Table 12.

TABLE 12

| ID # | Senktide Headshake Assay |
|---|---|
| 10 | Active |
| 15 | Active |

Active = a statistically significant (Mann-Whitney t-test (one-tailed)) reduction in the headshakes produced by senktide (5 mg/kg), in animals dosed with test compound 10 mg/kg po

### EXAMPLE 15

### In vivo Assay—Combination SMA and EPM Tests

Animals:

Male Long-Evans Hooded rats weighing 180 to 200 grams were purchased from Charles River Inc (Portage Mich.). The rats were housed in groups of four at an ambient temperature of 21 to 23° C. in a room with an automated 12/12 hour light/dark cycle. The rats had access to water and a commercial rodent food ad libitum. At the time of the experiment the rats weighed 220 to 350 grams.

The assay was run with test compound or vehicle administered to the animals at time zero. Fifty minutes after administration, the animals were tested in the SMA (Spontaneous Locomotor Activity), which was completed in 10 minutes. Immediately following SMA testing, the rats were moved and tested in the EPM (elevated Plus Maze), which was also completed in ten minutes. Test compounds were suspended in an aqueous vehicle (MC) comprised of 0.5% Methylcellulose and administered p.o.

Spontaneous Locomotor Activity (SMA) Test:

The test apparatus consisted of a plastic cubicle (40.6 cm, length; 40.6 cm, width; 30.5 cm, height) that was placed in the center of a main frame. Photocell sensors (8 beams from front to back and 8 beams from side to side) were built into the sides of the frame for monitoring horizontal movement. The photocells were located at right angles to each other, projecting horizontal infrared beams of light 5 cm apart and 2 cm above the floor to measure horizontal activity, and 5 cm apart and 14 cm above the floor to measure vertical activity. Rats were divided into groups (N=8 to 12). Test compound or vehicle was administered orally by gavage in a dose volume equivalent to 5 mL/kg. At 50 minutes after administration each rat was placed into a separate plastic cubicle and spontaneous exploratory activity was recorded for 10 minutes. Horizontal and vertical movements of the rats were recorded by counting the number of times the beams of light were interrupted (horizontal and vertical counts). Collection of the data and preliminary data analysis was automated. A drug-induced decrease in spontaneous horizontal or vertical motor activity was regarded as an indication of sedation.

Data Analysis (SMA):

A test compound was considered sedative in rats whose horizontal activity (HA) or vertical movements (VM, rearing) counts were significantly less than that in vehicle-treated rats. HA data were analyzed for statistical significance between drug and vehicle-treated groups that were administered either the vehicle or each dose of the test compound by a one-way analysis of variance. Then Dunnett's multiple comparison method was used to test for a reduction (p<0.05, 1-tailed) in the average number of HA counts or VM counts in drug-treated groups, compared to a concurrently run vehicle-treated group. If the probability was less than 5% (p<0.05) that a decrease in HA and/or VM in the drug-treated group compared to a concurrently run vehicle-treated group was due to chance, then the dose of the test compound was considered to have sedative activity. Mann-Whitney T-Test is used in cases where the distribution of the data is non-gaussian.

Elevated Plus Maze (EPM) Test:

The elevated plus maze (EPM) is the most widely used animal test of anxiety. The fully quantitative computerized EPM has validity as an anxiety model from the theoretical basis and pharmacological responses. The EPM also has high ecological validity since it studies the spontaneous behavioral patterns in response to interactions with the environment.

The procedure is based on the natural aversion of rodents to explore open and high places, as well as their innate tendency for thigmotaxis. When rats are placed on the elevated-plus maze, they have a normal tendency to remain in the enclosed arms of the maze and avoid venturing into the open arms. Animals treated with typical or atypical anxiolytics show an increase in the percentage of time spent (%Time) and/or the percentage of entries made (%Entries) into the open arms.

The test apparatus used consisted of a black plastic maze with two open arms and two arms with 40 cm high walls (enclosed arms) of equal length (50 cm) extending from the center at right angles, such that arms of similar type were opposite each other. Each plus-maze was elevated approximately 60 cm above the floor. Infrared photo-beams that crossed the entrance of each arm and the center of the maze detected the exploratory activity of an animal in the maze. Rats were divided into groups (N=8 to 12) and test compound or vehicle was administered orally by gavage in a dose volume equivalent to 5 mL/kg. One hour after dosing the rats were placed on an open arm of the plus-maze facing the center. The 10 minute test was initiated when the rat entered the center of the apparatus. Data collection was automated.

Data Analysis (EPM):

Anxiolytic activity of a test compound was quantified using two parameters: a) the percent of total time spent by a rat in one of the two open arms of the apparatus (% open arm time) which was calculated as follows:

$$\% Open Arm Time = ( \frac{( Time in Open Arms )}{( Total Time of Test Session )} ) \times 100 \%$$

and b) the number of times a rat entered the open arms relative to the total entries into all arms and the center area (% open arm entries), calculated as follows:

$$\% Open Arm Entries = ( \frac{( Entried into Open Arm )}{( Total Entries into Open Arms , Closed Arms , Center )} ) \times 100 \%$$

A test compound was considered active in rats whose % open arm time or % open arm entries was significantly greater than in rats that received vehicle. Data were analyzed for statistical significance between drug and vehicle-treated groups via one tailed Mann-Whitney T-Test. If the probability was less than 5% (p<0.05) that an increase in the % open arm time and/or % open arm entries in the drug-treated group compared to the vehicle-treated group was due to chance, then the dose of the test compound was considered active.

The total number of entries into all arms and the center of the EPM was recorded as part of the automated data collection in this test. This information (total entries) serves as a separate measure of spontaneous motor activity on the EPM. Compounds with sedative activity reduce the total number of entries in the Elevated-Plus Maze test.

A test compound was considered to have sedative activity in rats whose total entries was significantly less than in rats that received vehicle. Data were analyzed for statistical significance between drug and vehicle-treated groups via one tailed Mann-Whitney T-Test. If the probability was less than 5% (p<0.05) that a decrease in the total entries in the drug-treated group compared to the vehicle-treated group was due to chance, then the dose of the test compound was considered to be a dose at which the compound produces sedation.

Representative compounds of the instant invention were tested according to the spontaneous locomotor activity (SMA) and elevated plus maze (EPM) procedures described above, with results as listed in Table 13.

TABLE 13

| | Increase | Increase % | SMA | SMA |
|---|---|---|---|---|
| | % Open | Open Arm | Horizontal | Vertical |
| ID # | Arm Time | Entries | Activity | Movement |
| 10 | Active | Active | Increase | Increase |
| 15 | Active | Active | Increase | Increase |
| 75 | Active | Active | Increase | Increase |

Active = statistically significant (Mann Whitney U test p < 0.05) increase in open arm time or open arm entries at 10 mg/kg po
Increase = statistically significant (Mann Whitney U test p < 0.05) increase in horizontal activity and vertical movements indicating lack of sedation or motor impairment at 10 mg/kg po

### EXAMPLE 16

### In vivo Testing—Anti-emetic Test

The effectiveness of a test compound to inhibit emesis in the shrew were determined according to the procedure described in Darmani, N. A. Serotonin 5-HT3 receptor antagonists prevent cisplatin-induced emesis in Cryptosis parva: a new experimental model of emesis. J Neural. Transm. 1998, 105, 1143-1154.

Compound #10 was determined to be active in the cisplatin induced emesis in vivo test described above—i.e. the data showed a statistically significant reduction in the cisplatin induced retching behavior of shrews at a dosage of 20 mg/kg, administered subcutaneously.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. 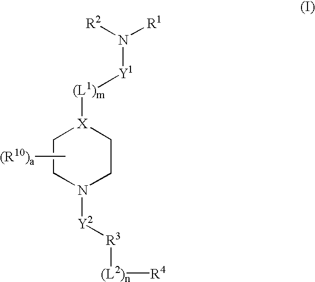
wherein
a is an integer selected from 0 to 2;
R¹⁰ is selected from the group consisting of C₁₋₆alkyl, aryl, C₃-C₈cycloalkyl, aralkyl, heteroaryl, heteroaryl-C₁₋₆alkyl, heterocycloalkyl and heterocycloalky-C₁₋₆alkyl; wherein the aryl, cycloalkyl, aralkyl, heteroaryl or heterocycloalkyl group may be optionally substituted with one to four substituents independently selected from halogen, hydroxy, C₁₋₆alkyl, halogenated C₁₋₆alkyl, C₁₋₆alkoxy, halogenatedC₁₋₆alkoxy, nitro, cyano, amino, C₁₋₄alkylamino, di(C₁₋₄alkyl)amino, C₁₋₆alkylsulfonyl, C₁₋₆alkoxysulfonyl or halogenated C₁₋₆alkylsulfonyl;
X is selected from the group consisting of CH, C(C₁-C₆alkyl) and N;
m is an integer selected from 0 and 1;
L¹ is selected from the group consisting of C₁-C₆alkyl;
Y¹ is selected from the group consisting of C(O) and C(S);
R¹ and R² are each independently selected from the group consisting of hydrogen, C₁-C₆alkyl, aryl, aralkyl, C₃-C₈cycloalkyl, C₃-C₈cycloalkyl-C₁₋₆alkyl, heteroaryl, heteroaryl-C₁₋₆alkyl, heterocycloalkyl and heterocycloalkyl-C₁₋₆alkyl; wherein the aryl, aralkyl, cycloalkyl, heteroaryl or heterocycloalkyl may be optionally substituted with one or more substituents independently selected from halogen, hydroxy, C₁-C₆alkyl, C₁-C₆alkoxy, halogenatedC₁-C₆alkyl, halogenatedC₁-C₆alkoxy, nitro, cyano, amino, C₁-C₄alkylamino, di(C₁-C₄alkyl)amino, heteroaryl or heterocycloalkyl;
alternatively, R¹ and R² may be taken together with the nitrogen atom to which they are bound to form a five to six membered monocyclic ring structure selected from the group consisting of pyrrolidinyl, piperidinyl, piperazinyl, morpholinyl and thiomorpholinyl;
Y² is selected from the group consisting of CH₂, C(O), C(S) and SO₂;
R³ is selected from the group consisting of aryl, aralkyl, C₃-C₈cycloalkyl, heteroaryl, heterocycloalkyl, C₃₋₈cycloalkyl-C₁₋₆alkyl and heterocycloalkyl-C₁₋₆alkyl; wherein the aryl, aralkyl, cycloalkyl, heteroaryl or heterocycloalkyl may be optionally substituted with one of more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino, di(C₁-C₄alkyl)amino or -(L²)ₙ-R⁴;
n is an integer selected from 0 and 1;
L² is selected from the group consisting of C₁-C₈alkyl, C₂-C₈alkenyl, C₂-C₈alkynyl, C(O), C(S), SO₂ and (A)₀₋₁—Q—(B)₀₋₁;
where A and B are each independently selected from C₁-C₆alkyl, C₂-C₆alkenyl and C₂-C₆alkynyl;
where Q is selected from the group consisting of NR⁵, O and S;
where R⁵ is selected from the group consisting of hydrogen, C₁-C₆alkyl, aryl, aralkyl, C₃₋₈cycloalkyl, heteroaryl, heterocycloalkyl, C(O)-C₁-C₆alkyl, C(O)-aryl, C(O)-aralkyl, C(O)-heteroaryl, C(O)-heterocycloalkyl, SO₂-C₁-C₆alkyl, SO₂-aryl, SO₂-aralkyl, SO₂-heteroaryl, SO₂-heterocycloalkyl and —CHR⁶R⁷;
wherein the aryl, aralkyl, cycloalkyl, heteroaryl or heterocycloalkyl may be optionally substituted with one or more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino;
where R⁶ and R⁷ are each independently selected from the group consisting of hydrogen, C₁₋₆alkyl, aryl, aralkyl, C₃₋₈cycloalkyl, heteroaryl, heterocycloalkyl, C(O)-C₁₋₆alkyl, C(O)aryl, C(O)-C₃₋₈cycloalkyl, C(O)-heteroaryl and C(O)-heterocycloalkyl; wherein the aryl, aralkyl, cycloalkyl, heteroaryl or heterocycloalkyl may be optionally substituted with one or more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino;
R⁴ is selected from the group consisting of aryl, aralkyl, C₃-C₈cycloalkyl, heteroaryl and heterocycloalkyl; wherein the aryl, aralkyl, cycloalkyl, heteroaryl or heterocycloalkyl may be optionally substituted with one or more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino;
provided that when a is 0; X is CH; m is 1; L¹ is CH₂; R³ is phenyl; n is 0; and R⁴ is phenyl, wherein the phenyl group may be optionally substituted with one substituent selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino, and wherein the R⁴ group is bonded to the R³ group in the para position;
then R¹ and R² are each independently selected from the group consisting of hydrogen, C₂-C₆alkyl, aryl, aralkyl, C₃-C₈cycloalkyl, C₃-C₈cycloalkyl-C₁₋₆alkyl, heteroaryl, heteroaryl-C₁₋₆alkyl, heterocycloalkyl and heterocycloalkyl-C₁₋₆alkyl; wherein the aryl, aralkyl, cycloalkyl, heteroaryl or heterocycloalkyl may be optionally substituted with one or more substituents independently selected from halogen, hydroxy, C₁-C₆alkyl, C₁-C₆alkoxy, halogenatedC₁-C₆alkyl, halogenatedC₁-C₆alkoxy, nitro, cyano, amino, C₁-C₄alkylamino, di(C₁-C₄alkyl)amino, heteroaryl or heterocycloalkyl;
alternatively, R¹ and R² may be taken together with the nitrogen atom to which they are bound to form a five to six membered monocyclic ring structure selected from the group consisting of pyrrolidinyl, piperidinyl, piperazinyl, morpholinyl and thiomorpholinyl;
provided further that when a is 0; X is N; m is 1; L¹ is CH₂; Y² is C(O)or C(S); n is 1; L² is 0; R⁴ is phenyl, wherein the phenyl may be optionally substituted with one or more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino; and R¹ and R² are each independently selected from the group consisting of hydrogen and C₁₋₆alkyl;
then R³ is selected from the group consisting of aryl, aralkyl, C₃-C₈cycloalkyl, heteroaryl other than thienopyridinyl, heterocycloalkyl, C₃₋₈cycloalkyl-C₁₋₆alkyl and heterocycloalkyl-C₁₋₆alkyl; wherein the aryl, aralkyl, cycloalkyl, heteroaryl or heterocycloalkyl may be optionally substituted with one of more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino, di(C₁-C₄alkyl)amino or —(L²)ₙ—R⁴;
provided further that when a is 0; X is N; m is 1; L¹ is CH₂; Y² is C(O) or C(S); n is 0; R¹ and R² are taken together with the nitrogen to which they are bound to form pyrrolidinyl; and R⁴ is pyridyl;
then R³ is selected from the group consisting of aryl, aralkyl, C₃-C₈cycloalkyl, heteroaryl, heterocycloalkyl other than thiazolidinyl; C₃₋₈cycloalkyl-C₁₋₆alkyl and heterocycloalkyl-C₁₋₆alkyl; wherein the aryl, aralkyl, cycloalkyl, heteroaryl or heterocycloalkyl may be optionally substituted with one of more substituents independently selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino, di(C₁-C₄alkyl)amino or —(L²)ₙ—R⁴;
provided further that when R¹ and R² are each independently selected from the group consisting of hydrogen and C₁₋₆alkyl, or R¹ and R² are taken together with the nitrogen atom to which they are bound to form morpholinyl or pyrrolidinyl; a is 0; X is N; m is 1; L¹ is CH₂; Y² is C(O) or C(S); n is 0; and R⁴ is phenyl, wherein the phenyl is optionally substituted with one or more substituents independently selected from C₁-C₆alkyl, C₁-C₆alkoxy, halogenatedC₁-C₆alkyl, halogenatedC₁-C₆alkoxy or nitro;
then R³ is selected from the group consisting of aryl, aralkyl, heteroaryl, heterocycloalkyl, C₃₋₈cycloalkyl-C₁₋₆alkyl and heterocycloalkyl-C₁₋₆alkyl; wherein the aryl, aralkyl, cycloalkyl, heteroaryl or heterocycloalkyl may be optionally substituted with one substituent selected from halogen, hydroxy, C₁-C₆alkyl, C₁-C₆ alkoxy, halogenatedC₁-C₆alkyl, halogenatedC₁-C₆alkoxy, nitro, cyano, amino, C₁-C₄alkylamino or di(C₁-C₄alkyl)amino;
and pharmaceutically acceptable salts thereof.

2. A compound as in claim 1 of the formula 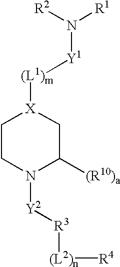
wherein
a is 0to 1;
R¹⁰ is selected from the group consisting of C₁-C₄alkyl and aralkyl;
X is selected from the group consisting of CH, C(methyl) and N;
m is an integer selected from 0 or 1;
L¹ is selected from the group consisting of C₁-C₄ alkyl;
Y¹ is C(O);
R¹ and R² are each independently selected from the group consisting of hydrogen, C₁₋₄alkyl, aryl, aralkyl, C₃₋₈cycloalkyl-C₁-C₄alkyl, heteroaryl and heterocycloalkyl; wherein the aryl, aralkyl or heteroaryl may be optionally substituted with one to two substituents independently selected from halogen, hydroxy, C₁-C₄alkyl, C₁-C₄alkoxy, trifluoromethyl, trifluoromethoxy, C₁-C₄alkylamino, di(C₁-C₄alkyl)amino or heterocycloalkyl;
alternatively, R¹ and R² may be taken together with the nitrogen atom to which they are bound to form a five to six membered monocyclic ring structure selected from the group consisting of pyrrolidinyl, piperidinyl, piperazinyl, morpholinyl and thiomorpholinyl; Y² is C(O);
R³ is selected from the group consisting of aryl and heteroaryl; wherein the aryl or heteroaryl may be optionally substituted with one to two substituents independently selected from C₁-C₄alkyl, trifluoromethyl or —(L²)ₙ—R⁴;
n is an integer selected from 0 or 1;
L² is selected from the group consisting of C₁-C₆alkyl, C₂-C₆alkenyl, C₂-C₆alkynyl and (A)₀₋₁—Q—(B)₀₋₁;
where A and B are each independently selected from C₁-C₄alkyl;
where Q is selected from the group consisting of NR⁵, O and S;
where R⁵ is selected from the group consisting of hydrogen, C₁-C₄alkyl, C(O)-C₁-C₆alkyl, C(O)-aryl, C(O)-aralkyl, C(O)-heteroaryl, C(O)-heterocycloalkyl and —CHR⁶R⁷; wherein the aryl, aralkyl, cycloalkyl, heteroaryl or heterocycloalkyl may be optionally substituted with one to two substituents independently selected from halogen, C₁-C₄alkyl, C₁-C₄alkoxy, trifluoromethyl, trifluoromethoxy, nitro, cyano, amino, C₁-C₄alkylamino or di(C₁-C₄alkyl)amino;
where R⁶ and R⁷ are each independently selected from the group consisting of hydrogen, C₁₋₄alkyl, aryl, aralkyl, C₃₋₈cycloalkyl, heteroaryl, heterocycloalkyl, C(O)-C₁₋₆alkyl, C(O)aryl, C(O)-C₃₋₈cycloalkyl, C(O)-heteroaryl and C(O)-heterocycloalkyl; wherein the aryl, aralkyl, cycloalkyl, heteroaryl or heterocycloalkyl may be optionally substituted with one to two substituents independently selected from halogen, hydroxy, C₁-C₄alkyl, C₁-C₄alkoxy, trifluoromethyl, trifluoromethoxy, nitro, cyano, amino, C₁-C₄alkylamino or di(C₁-C₄alkyl)amino;
R⁴ is selected from the group consisting of aryl, heteroaryl and heterocycloalkyl; wherein the aryl group may be optionally substituted with one to two substituents independently selected from halogen, hydroxy, C₁-C₄alkyl, C₁₋₄alkoxy, trifluoromethyl or amino;
provided that when a is 0; X is CH; m is 1; L¹ is CH₂; R³ is phenyl; n is 0; and R⁴ is phenyl, wherein the phenyl group may be optionally substituted with one substituent selected from halogen, hydroxy, C₁-C₄alkyl, C₁-C₄alkoxy, trifluoromethyl or amino, and wherein the R⁴ group is bonded to the R³ group in the para position;
then R¹ and R² are each independently selected from the group consisting of hydrogen, C₂₋₄alkyl, aryl, aralkyl, C₃₋₈cycloalkyl-C₁-C₄alkyl, heteroaryl and heterocycloalkyl; wherein the aryl, aralkyl or heteroaryl may be optionally substituted with one to two substituents independently selected from halogen, hydroxy, C₁-C₄alkyl, C₁-C₄alkoxy, trifluoromethyl, trifluoromethoxy, C₁-C₄alkylamino, di(C₁-C₄alkyl)amino or heterocycloalkyl;
alternatively, R¹ and R² may be taken together with the nitrogen atom to which they are bound to form a five to six membered monocyclic ring structure selected from the group consisting of pyrrolidinyl, piperidinyl, piperazinyl, morpholinyl and thiomorpholinyl;
provided further that when a is 0; X is N; m is 1; L¹ is CH₂; Y² is C(O); n is 1; L² is 0; R⁴ is phenyl, wherein the phenyl may be optionally substituted with one to two substituents independently selected from halogen, hydroxy, C₁-C₄alkyl, C₁-C₄ alkoxy, trifluoromethyl or amino; and R¹ and R² are each independently selected from the group consisting of hydrogen and C₁₋₄alkyl;
then R³ is selected from the group consisting of aryl and heteroaryl other than thienopyridinyl; wherein the aryl or heteroaryl may be optionally substituted with one to two substituents independently selected from C₁-C₄alkyl, trifluoromethyl or —(L²)ₙ—R⁴;
provided further that when R¹ and R² are each independently selected from the group consisting of hydrogen and C₁₋₄alkyl, or R¹ and R² are taken together with the nitrogen atom to which they are bound to form morpholinyl or pyrrolidinyl; a is 0; X is N; m is 1; L¹ is CH₂; Y² is C(O); n is 0; and R⁴ is phenyl, wherein the phenyl is optionally substituted with one or two substituents independently selected from C₁-C₄alkyl, C₁-C₄alkoxy or trifluoromethyl;
then R³ is selected from the group consisting of aryl and heteroaryl; wherein the aryl or heteroaryl may be optionally substituted with one substituent selected from C₁-C₄alkyl or trifluoromethyl;
and pharmaceutically acceptable salts thereof.

3. A compound as in claim 2 wherein
X is selected from the group consisting of CH and N;
m is 1;
R¹ is selected from the group consisting of hydrogen and C₁₋₄alkyl;
R² is selected from the group consisting of C₁₋₄alkyl, aryl, aralkyl, C₃₋₈cycloalkyl-C₁₋₄alkyl and heteroaryl; wherein the aryl or aralkyl may be optionally substituted with one to two substituents independently selected from halogen, hydroxy, C₁-C₄alkyl, C₁-C₄alkoxy, trifluoromethyl, trifluoromethoxy, di(C₁-C₄alkyl)amino or heterocycloalkyl;
alternatively, R¹ and R² may be taken together with the nitrogen atom to which they are bound to form a five to six membered monocyclic ring structure selected from the group consisting of pyrrolidinyl, piperidinyl and morpholinyl;
R³ is selected from the group consisting of aryl and heteroaryl; wherein the aryl or heteroaryl may be optionally substituted with a substituent selected from C₁-C₄alkyl or trifluoromethyl;
L² is selected from the group consisting of C₁-C₄alkyl, C₂-C₆alkenyl, C₂-C₆alkynyl, NH-C₁₋₄alkyl, C₁₋₄alkyl-N(C₁₋₄alkyl)-C₁₋₄alkyl and C₁₋₄alkyl-N(C(O)C_{1 4}alkyl)-C₁₋₄alkyl;
provided that when a is 0; X is CH; L¹ is CH₂; R³ is phenyl; n is 0; and R⁴ is phenyl, wherein the phenyl group may be optionally substituted with one substituent selected from halogen, hydroxy, C₁-C₄alkyl, C₁-C₄alkoxy, trifluoromethyl or amino, and wherein the R⁴ group is bonded to the R³ group in the para position;
then R¹ is selected from the group consisting of hydrogen and C₂₋₄alkyl;
R² is selected from the group consisting of C₂₋₄alkyl, aryl, aralkyl, C₃₋₈cycloalkyl-C₁₋₄alkyl and heteroaryl; wherein the aryl or aralkyl may be optionally substituted with one to two substituents independently selected from halogen, hydroxy, C₁-C₄alkyl, C₁-C₄alkoxy, trifluoromethyl, trifluoromethoxy, di(C₁-C₄alkyl)amino or heterocycloalkyl;
alternatively, R¹ and R² are taken together with the nitrogen atom to which they are bound to form a five to six membered monocyclic ring structure selected from the group consisting of pyrrolidinyl, piperidinyl and morpholinyl;
and pharmaceutically acceptable salts thereof.

4. A compound as in claim 3 wherein
R¹⁰ is selected from the group consisting of methyl and benzyl;
L¹ is selected from the group consisting of CH₂ and CH₂CH₂;
R² is selected from the group consisting of —CH₂—(3-trifluoromethyl phenyl), -CH₂-cyclohexyl, -CH₂-(3,5-dimethoxyphenyl), —CH₂—(4-trifluoromethylphenyl), -CH₂-(3,5-ditrifluoromethylphenyl), 3-trifluoromethoxyphenyl, -CH₂-(4-dimethylaminophenyl), phenyl, benzyl, 2-fluorophenyl, 4-fluorophenyl, 2,4-difluorophenyl, 2,6-difluorophenyl, 4-hydroxyphenyl, 4-dimethylamino-phenyl, 2-pyridyl, 3-pyridyl, 4-pyridyl, 4-pyridyl-methyl, 4-morpholinyl-phenyl, 4-piperidinyl-phenyl, methyl, isopropyl, 4-methoxyphenyl, 4-trifluoromethylphenyl, 2-pyrimidinyl, 4-pyrimidinyl, 5-quinolinyl, 6-quinolinyl, and 8-quinolinyl;
alternatively, R¹ and R² are taken together with the nitrogen atom to which they are bound to form a five to six membered monocyclic ring structure selected from the group consisting of pyrrolidinyl, piperidinyl and morpholinyl;
R³ is selected from the group consisting of phenyl, methylphenyl, trifluoromethylphenyl, 4-oxazolyl and 3-(2-trifluoromethyl-furyl);
L² is selected from the group consisting of 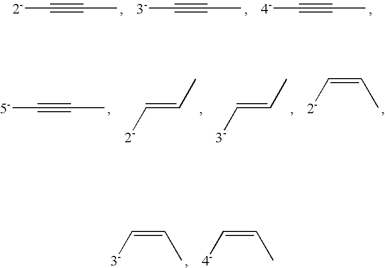
2—CH₂CH₂, 3—CH₂—CH₂, 4—CH₂—CH₂, NH—CH₂, CH₂-N(CH₃)-CH₂, CH₂—N(CH₃)—CH₂CH₂, CH₂—N(C(O)CH₃)—CH₂ and CH₂—N(C(O)CH₃)—CH₂CH₂;
R⁴ is selected from the group consisting of phenyl, 1-naphthyl, 2-pyridyl, 3-pyridyl, 4-pyridyl, 3-hydroxyphenyl, 2-methylphenyl, 3-aminophenyl, 4-methoxyphenyl, 4-chlorophenyl, 2-thienyl, 3-thienyl, 3,5-di(trifluoromethyl)-phenyl, 1-imidazolyl, 2-benzimidazolyl, 1-pyrrolidinyl, 2-furyl and 2-tetrahydrofuryl;
provided that when a is 0; X is CH; L¹ is CH₂; R³ is phenyl; n is 0; and R⁴ is phenyl, 4-chlorophenyl, 3-hydroxyphenyl, 2-methylphenyl, 4-methoxyphenyl or 3-aminophenyl; and wherein the R⁴ group is bonded to the R³ group in the para position;
then R¹ is selected from the group consisting of hydrogen and C₂₋₄alkyl;
R² is selected from the group consisting of -CH₂-(3-trifluoromethyl phenyl), -CH₂-cyclohexyl, -CH₂-(3,5-dimethoxyphenyl), -CH₂-(4-trifluoromethylphenyl), -CH₂-(3,5-ditrifluoromethylphenyl), 3-trifluoromethoxyphenyl, -CH₂-(4-dimethylaminophenyl), phenyl, benzyl, 2-fluorophenyl, 4-fluorophenyl, 2,4-difluorophenyl, 2,6-difluorophenyl, 4-hydroxyphenyl, 4-dimethylamino-phenyl, 2-pyridyl, 3-pyridyl, 4-pyridyl, 4-pyridyl-methyl, 4-morpholinyl-phenyl, 4-piperidinyl-phenyl, isopropyl, 4-methoxyphenyl, 4-trifluoromethylphenyl, 2-pyrimidinyl, 4-pyrimidinyl, 5-quinolinyl, 6-quinolinyl, and 8-quinolinyl;
alternatively, R¹ and R² are taken together with the nitrogen atom to which they are bound to form a five to six membered monocyclic ring structure selected from the group consisting of pyrrolidinyl, piperidinyl and morpholinyl;
and pharmaceutically acceptable salts thereof.

5. A compound as in claim 4 of the formula 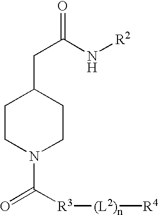
wherein
R² is selected from the group consisting of -CH₂-(3-trifluoromethylphenyl), -CH₂-cyclohexyl, -CH₂-(3,5-dimethoxyphenyl), -CH₂-(4-trifluoromethylphenyl), -CH₂-(3,5-ditrifluoromethylphenyl), -CH₂-(4-dimethylaminophenyl), phenyl, 2-fluorophenyl, 4-fluorophenyl, 2,4-difluorophenyl, 2,6-difluorophenyl, 3-trifluoromethylphenyl, 4-trifluoromethylphenyl, 4-hydroxyphenyl, 4-methoxyphenyl, benzyl, 3-pyridyl, 4-pyridyl, 2-pyrimidinyl, 4-pyrimidinyl, 5-quinolinyl, 6-quinolinyl, 8-quinolinyl, 4-(dimethylamino)-phenyl, 4-morpholinyl-phenyl, 4-pyridyl-methyl, and 4-piperidinyl-phenyl;
L² is selected from the group consisting of 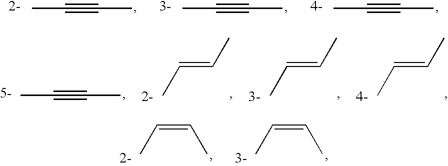
2—CH₂CH₂, 3—CH₂—CH₂, 4—CH₂—CH₂, NH—CH₂, 4—(CH₂—N(CH₃)—CH₂), 4—(CH₂—N(CH₃)—CH₂CH₂), 4—(CH₂—N(C(O)CH₃)—CH₂) and 4—(CH₂—N(C(O)CH₃)—CH₂);
R⁴ is selected from the group consisting of phenyl, 3-phenyl; 5-phenyl, 4-chlorophenyl, 3-hydroxyphenyl, 3-(2-methylphenyl), 3-(3-aminophenyl), 2-pyridyl, 3-pyridyl, 3-(3-pyridyl), 4-pyridyl, 3-(3-thienyl), 3,5-di(trifluoromethyl)phenyl, 1-pyrrolidinyl, 2-furyl, 1-naphthyl, 2-thienyl, 1-imidazolyl, 2-benzimidazolyl and 2-tetrahydrofuryl;
and pharmaceutically acceptable salts thereof.

6. A compound as in claim 4 of the formula 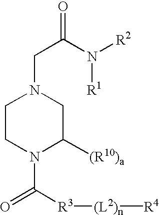
wherein;
R¹ is selected from the group consisting of hydrogen and methyl;
R² is selected from the group consisting of isopropyl, phenyl, 2-fluorophenyl, 4-fluorophenyl, 2,4-difluorophenyl, 2,6-difluorophenyl, 3-pyridyl, 1-pyrrolidinyl, 4-dimethylamino-phenyl and 4-morpholinyl-phenyl;
alternatively R¹ and R² are taken together with the nitrogen atom to which they are bound to form a five to six membered ring structure selected from the group consisting of 1-pyrrolidinyl, 1-piperidinyl and 1-morpholinyl;
R³ is selected from the group consisting of phenyl and 3-(2-trifluoromethyl-furyl);
n is an integer from 0 to 1;
L² is selected from the group consisting of 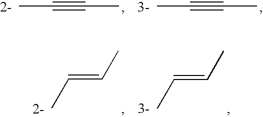
3—CH₂—CH₂ and NH—CH₂;
R⁴ is selected from the group consisting of phenyl, 4-methoxyphenyl, 4-chlorophenyl, 2-pyridyl, 3-pyridyl, 4-pyridyl and 3,5-di(trifluoromethyl)phenyl;
and pharmaceutically acceptable salts thereof.

7. A compound as in claim 4 selected from the group consisting of
N-phenyl-1-[3-(2-pyridinylethynyl)benzoyl]-4-piperidineacetamide;
N-(2,4-difluorophenyl)-1-[3-(2-pyridinylethynyl)benzoyl]-4-piperidineacetamide;
N-phenyl-4-[2-[(E)-2-(2-pyridinyl)ethenyl]benzoyl]-1-piperazineacetamide;
N-phenyl-4-[3-(2-pyridinylethynyl)benzoyl]-1-piperazineacetamide;
N-(4-hydroxyphenyl)-1-[3-(2-pyridinylethynyl)benzoyl]-4-piperidineacetamide;
and pharmaceutically acceptable salts thereof.

8. A compound as in claim 4 of the formula 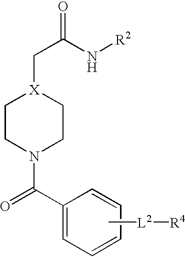
X is selected from the group consisting of CH and N;
R² is selected from the group consisting of phenyl, 4-hydroxyphenyl, 2-fluorophenyl, 4-fluorophenyl, and 2,4-difluorophenyl;
L² is selected from the group consisting of 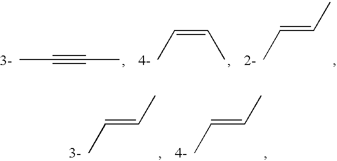
4—(CH₂—N(CH₃)—CH₂CH₂), 4—(CH₂—N(CH₃)—CH₂) and 3—NH—CH₂;
R⁴ is selected from the group consisting of 2-pyridyl, 4-pyridyl, 4-pyrrolidinyl, 2-furyl, 1-naphthyl and 3,5-di(trifluoromethyl)phenyl;
and pharmaceutically acceptable salts thereof.

9. A compound as in claim 8 wherein X is CH; R² is phenyl; L² is 3- 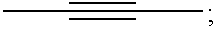
R⁴ is 2-pyridyl and pharmaceutically acceptable salts thereof.

10. A pharmaceutical composition comprising a pharmaceutically acceptable carrier and a compound of claim 1.

11. A pharmaceutical composition made by mixing a compound of claim 1 and a pharmaceutically acceptable carrier.

12. A process for making a pharmaceutical composition comprising mixing a compound of claim 1 and a pharmaceutically acceptable carrier.

13. A method of treating a nervous system disorder in a subject in need thereof comprising administering to the subject a therapeutically effective amount of the compound of claim 1.

14. The method of claim 10, wherein the nervous system disorder is selected from the group consisting of depression, dementia, schizophrenia, bipolar disorders, anxiety, emesis, acute pain, neuropathic pain, itching, migraine and movement disorders.

15. A method of treating nervous system a disorder in a subject in need thereof comprising administering to the subject a therapeutically effective amount of the composition of claim 10.

16. A method of treating a nervous system disorder selected from the group consisting of depression and anxiety in a subject in need thereof comprising administering to the subject a therapeutically effective amount of the compound of claim 1.

17. A method of treating a nervous system disorder selected from the group consisting of depression and anxiety in a subject in need thereof comprising administering to the subject a therapeutically effective amount of the pharmaceutical composition of claim 10.

18. A method of treating a nervous system disorder selected from the group consisting of depression and anxiety in a subject in need thereof comprising administering to the subject a therapeutically effective amount of the compound of claim 9.
